(19) United States
(12) Patent Application Publication
(10) Pub. No.: US 2016/0175460 A1
(43) Pub. Date: Jun. 23, 2016

(54) ENGINEERED ANTI-DLL3 CONJUGATES AND METHODS OF USE

(71) Applicant: Stemcentrx, Inc., South San Francisco, CA (US)

(72) Inventors: William Robert ARATHOON, Los Altos Hills, CA (US); Ishai PADAWER, San Francisco, CA (US); Luis Antonio CANO, Oakland, CA (US); Vikram Natwarsinhji SISODIYA, San Francisco,, CA (US); Karthik Narayan MANI, San Francisco, DC (US); David LIU, San Francisco, CA (US)

(73) Assignee: Stemcentrx, Inc., South San Francisco, CA (US)

(21) Appl. No.: 15/056,893

(22) Filed: Feb. 29, 2016

Related U.S. Application Data
(63) Continuation of application No. PCT/US2014/053304, filed on Aug. 28, 2014.
(60) Provisional application No. 61/871,173, filed on Aug. 28, 2013.

(51) Int. Cl. A61K 47/48; C07K 16/30; C07K 16/28

(52) U.S. Cl. CPC A61K 47/48592; A61K 47/48561; C07K 16/28; C07K 16/30; C07K 16/3023; A61K 47/48569; A61K 47/48384; C07K 2317/24; C07K 2317/76; A61K 2039/505

(57) ABSTRACT

Provided are novel antibody drug conjugates (ADCs), and methods of using such ADCs to treat proliferative disorders.

### CROSS REFERENCED APPLICATIONS

This application claims the benefit of U.S. Provisional Application No. 61/871,173 filed on Aug. 28, 2013 which is incorporated herein by reference in its entirety.

### SEQUENCE LISTING

The instant application contains a sequence listing which has been submitted in ASCII format via EFS-Web and is hereby incorporated by reference in its entirety. Said ASCII copy, created on Aug. 28, 2014, is named “sc1604pct_S69697_1220WO_{—} SEQL_{—} 082814.txt” and is 609 KB (624,275 bytes) in size.

### FIELD OF THE INVENTION

This application generally relates to novel compounds comprising anti-DLL3 antibodies or immunoreactive fragments thereof having one or more unpaired cysteine residues conjugated to pyrrolobenzodiazepines (PBDs) and use of the same for the treatment or prophylaxis of cancer and any recurrence or metastasis thereof.

### BACKGROUND OF THE INVENTION

Many commonly employed cancer therapeutics tend to induce substantial toxicity due to their inability to selectively target proliferating tumor cells. Rather, these traditional chemotherapeutic agents act non-specifically and often damage or eliminate normally proliferating healthy tissue along with the tumor cells. Quite often this unintended cytotoxicity limits the dosage or regimen that the patient can endure, thereby effectively limiting the therapeutic index of the agent. As a result, numerous attempts have made to target cytotoxic therapeutic agents to the tumor site with varying degrees of success. One promising area of research has involved the use of antibodies to direct cytotoxic agents to the tumor so as to provide therapeutically effective localized drug concentrations.

In this regard it has long been recognized that the use of targeting monoclonal antibodies (“mAbs”) conjugated to selected cytotoxic agents provides for the delivery of relatively high levels of such cytotoxic payloads directly to the tumor site while reducing the exposure of normal tissue to the same. While the use of such antibody drug conjugates (“ADCs”) has been extensively explored in a laboratory or preclinical setting, their practical use in the clinic is much more limited. In certain cases these limitations were the result of combining weak or ineffective toxins with tumor targeting molecules that were not sufficiently selective or failed to effectively associate with the tumor. In other instances the molecular constructs proved to be unstable upon administration or were cleared from the bloodstream too quickly to accumulate at the tumor site in therapeutically significant concentrations. While such instability may be the result of linker selection or conjugation procedures, it may also be the result of overloading the targeting antibody with toxic payloads (i.e., the drug to antibody ratio or “DAR” is too high) thereby creating an unstable conjugate species in the drug preparation. In some instances construct instability, whether from design or from unstable DAR species, has resulted in unacceptable non-specific toxicity as the potent cytotoxic payload is prematurely leached from the drug conjugate and accumulates at the site of injection or in critical organs as the body attempts to clear the untargeted payload. As such, relatively few ADCs have been approved by the Federal Drug Administration to date though several such compounds are presently in clinical trials. Accordingly, there remains a need for stable, relatively homogeneous antibody drug conjugate preparations that exhibit a favorable therapeutic index.

### SUMMARY OF THE INVENTION

These and other objectives are provided for by the present invention which, in a broad sense, is directed to novel methods, compounds, compositions and articles of manufacture that may be used in the treatment of DLL3 associated disorders (e.g., proliferative disorders or neoplastic disorders). To that end, the present invention provides novel delta-like ligand 3 (or DLL3) site-specific conjugates comprising pyrrolobenzodiazepine (“PBD”) payloads that effectively target tumor cells and/or cancer stem cells and may be used to treat patients suffering from a wide variety of malignancies. As will be discussed in detail below, the disclosed site-specific conjugates comprise engineered anti-DLL3 antibody constructs having one or more unpaired cysteines which may be preferentially conjugated to PBD payloads using novel selective reduction techniques. Such site-specific conjugate preparations are relatively stable when compared with conventional conjugated preparations and substantially homogenous as to average DAR distribution. As shown in the appended Examples the stability and homogeneity of disclosed anti-DLL3 site-specific conjugate preparations (regarding both average DAR distribution and PBD positioning) provide for a favorable toxicity profile that contributes to an improved therapeutic index

Accordingly, in one embodiment the present invention comprises an antibody drug conjugate of the formula:

Ab-[L-D]n or a pharmaceutically acceptable salt thereof wherein
a) Ab comprises a DLL3 antibody comprising one or more unpaired cysteines;
b) L comprises an optional linker;
c) D comprises a PBD; and
d) n is an integer from about 1 to about 8.

Any anti-DLL3 antibody, which specifically binds to human DLL3, may be used as the antibody portion, Ab, of antibody drug conjugates as disclosed herein. For example, in various aspects of the invention, the DLL3 antibody is a monoclonal antibody, a humanized antibody, or a CDR grafted antibody. In some aspects of the invention, the DLL3 antibody comprises any one of hSC16.13, hSC16.15, hSC16.25, hSC16.34 and hSC16.56, or an antibody that competes for binding to human DLL3 with any one of hSC16.13, hSC16.15, hSC16.25, hSC16.34 and hSC16.56. DLL3 antibodies used to prepare antibody drug conjugates can include any suitable constant region, including for example, an IgG1 heavy chain constant region and/or a kappa light chain constant region. In some aspects, the DLL3 antibodies used to prepare antibody drug conjugates are further characterized as internalizing antibodies.

In one embodiment the invention is directed to anti-DLL3 site-specific engineered conjugates comprising at least one unpaired cysteine residue. Those of skill in the art will appreciate that the unpaired interchain cysteine residues provide site(s) for the selective and controlled conjugation of pharmaceutically active moieties to produce ADCs in accordance with the teachings herein. For example, DLL3 antibodies useful for site specific conjugation of a drug will comprise one or more unpaired cysteines, for example, two or more unpaired cysteines, three or more unpaired cysteines, four or more unpaired cysteines, etc. The unpaired cysteines may be located on the light chain or the heavy chain. In some embodiments the unpaired cysteine residue(s) will comprise heavy/light chain interchain residues as opposed to heavy/heavy chain interchain residues.

In particular aspects of the invention, the DLL3 antibody comprises a light chain having an unpaired cysteine at position C214, and/or a heavy chain having an unpaired cysteine at position C220 (numbering according to the EU index of Kabat). For example, the DLL3 antibody can be a site-specific engineered IgG1 isotype antibody wherein the C214 residue of the light chain is substituted with another residue or deleted. In a related embodiment the C214 residue of said engineered antibody can be substituted to a serine. As another example, the invention provides a DLL3 antibody wherein the C220 residue of an IgG1 or IgG2 heavy chain is substituted with another residue or deleted, or wherein the C220 residue of an IgG1 or IgG2 heavy chain is substituted with a serine.

In some aspects of the invention, the drugs used to prepare antibody drug conjugates are pyrrolbenzodiazepines (PBDs), for example PBD1, PBD2, PBD3, PBD 4, and PBD 5, as disclosed herein. In other aspects, the invention provides an ADC comprising an engineered antibody comprising at least two unpaired interchain cysteine residues and PBDs conjugated to the at least two unpaired interchain cysteine residues.

A linker may or may not be used to associate the DLL3 antibody with a drug to prepare an antibody drug conjugate. A linker is optionally used as appropriate based upon the selection of a particular drug. In some aspects of the invention, the linker is a cleavable linker, such as for example, a dipeptide linker. In particular aspects of the invention, a cleavable linker is used to associate PBD1, PBD2, PBD3, PBD 4, or PBD 5 with the DLL3 antibody. In other aspects of the invention, an antibody drug conjugate comprises ADC 1, ADC 2, ADC 3, ADC 4, or ADC 5, as described herein, wherein the antibody (Ab) is an engineered DLL3 antibody.

In addition to the foregoing antibody drug conjugates, the invention further provides pharmaceutical compositions generally comprising the disclosed ADCs and methods of using such ADCs to diagnose or treat disorders, including cancer, in a patient. For example, the invention provides a method of treating cancer comprising administering to a subject a pharmaceutical composition comprising an ADC of the instant invention. In a particular aspect of the invention, the disclosed ADCs are useful for the treatment of small cell lung cancer.

In a related embodiment the invention is directed to a method of killing, reducing the frequency or inhibiting the proliferation of tumor cells or tumorigenic cells comprising treating said tumor cells or tumorigenic cells with an ADC of the instant invention.

In another embodiment the present invention comprises a method of preparing an antibody drug conjugate of the invention comprising the steps of:
a) providing an anti-DLL3 antibody comprising an unpaired cysteine;
b) selectively reducing the anti-DLL3 antibody; and
c) conjugating the selectively reduced anti-DLL3 antibody to a PBD.

In a related aspect the invention provides a method of preparing an ADC comprising: culturing a host cell expressing an engineered antibody; recovering said engineered antibody from said cultured host cell or culture medium; selectively reducing said engineered antibody; and conjugating a PBD said engineered antibody.

In a further aspect the invention provides an article of manufacture comprising an ADC of the instant invention; a container; and a package insert or label indicating that the compound can be used to treat cancer characterized by the expression of at least one antigen.

### BRIEF DESCRIPTION OF THE FIGURES

FIG. 1 is a depiction of the structure of the human IgG1 antibody showing the intrachain and interchain disulfide bonds.

FIGS. 2A and 2B provide, in a tabular form, contiguous amino acid sequences (SEQ ID NOS: 389-407, odd numbers) of light and heavy chain variable regions of a number of humanized exemplary DLL3 antibodies compatible with the disclosed antibody drug conjugates isolated, cloned and engineered as described in the Examples herein.

FIGS. 3A and 3B provide amino acid sequences of light and heavy chains (SEQ ID NOS: 14-19) of exemplary site-specific anti-DLL3 antibodies produced in accordance with the instant teachings.

FIG. 4 is a schematic representation depicting the process of conjugating an engineered antibody to a cytotoxin.

FIG. 5 is a graphical representation showing the conjugation rates of site-specific antibody light and heavy chains conjugated using reducing agents as determined using RP-HPLC.

FIG. 6 is a graphical representation showing the DAR distribution of site-specific antibody constructs conjugated using reducing agents as determined using HIC.

FIG. 7 shows the conjugation rates of site-specific antibody light and heavy chains conjugated using stabilizing agents or reducing agents as determined using RP-HPLC.

FIG. 8 a graphical representation showing the DAR distribution of site-specific antibody constructs conjugated using stabilization or reducing agents as determined using HIC.

FIG. 9 shows the DAR distribution of site-specific antibody constructs conjugated using stabilization and/or mild reducing agents as determined using HIC.

FIGS. 10A and 10B depict DAR distribution of site-specific antibody constructs conjugated using various stabilization agents as determined using HIC.

FIGS. 11A and 11B depict conjugation rates and DAR distribution of site-specific antibody constructs conjugated and purified as set forth herein.

FIGS. 12A and 12B show binding properties of unconjugated and conjugated site-specific constructs fabricated as set forth herein.

FIG. 13 graphically depicts the rate of in vitro cell killing provided by site-specific ADCs fabricated as set forth herein.

FIGS. 14A and 14B illustrate the enhanced stability of site-specific ADCs provided by the instant invention.

FIGS. 15A-15C graphically demonstrate the in vivo efficacy provided by the site-specific conjugates of the instant invention.

FIGS. 16A-16D illustrate the reduced toxicity provided by the site-specific conjugates of the instant invention.

### DETAILED DESCRIPTION OF THE INVENTION

### I. Introduction

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described. Finally, for the purposes of the instant disclosure all identifying sequence Accession numbers may be found in the NCBI Reference Sequence (RefSeq) database and/or the NCBI GenBank archival sequence database unless otherwise noted.

The site-specific anti-DLL3 PBD conjugates of the instant invention have been found to exhibit favorable characteristics that make them particularly suitable for use as therapeutic compounds and compositions. In this regard the conjugates immunospecifically react with a determinant, delta-like ligand 3 or DLL3 that has been found to be associated with various proliferative disorders and shown to be a good therapeutic target. Additionally, the constructs of the instant invention provide for selective conjugation at specific cysteine positions derived from disrupted native disulfide bond(s) obtained through molecular engineering techniques. This engineering of the antibodies provides for regulated stoichiometric conjugation that allows the drug to antibody ratio (“DAR”) to largely be fixed with precision resulting in the generation of largely DAR homogeneous preparations. Moreover the disclosed site-specific constructs further provide preparations that are substantially homogeneous with regard to the position of the payload on the antibody. Selective conjugation of the engineered constructs using stabilization agents as described herein increases the desired DAR species percentage and, along with the fabricated unpaired cysteine site, imparts conjugate stability and homogeneity that reduces non-specific toxicity caused by the inadvertent leaching of PBD. This reduction in toxicity provided by selective conjugation of unpaired cysteines and the relative homogeneity (both in conjugation positions and DAR) of the preparations also provides for an enhanced therapeutic index that allows for increased PBD payload levels at the tumor site. Additionally, the resulting site-specific anti-DLL3 PBD conjugates may optionally be purified using various chromatographic methodology to provide highly homogeneous site-specific conjugate preparations comprising desired DAR species (e.g., DAR=2) of greater than 75%, 80%, 85%, 90% or even 95%. Such conjugate homogeneity may further increase the therapeutic index of the disclosed preparations by limiting unwanted higher DAR conjugate impurities (which may be relatively unstable) that could increase toxicity.

It will be appreciated that the favorable properties exhibited by the disclosed engineered conjugate preparations is predicated, at least in part, on the ability to specifically direct the conjugation and largely limit the fabricated conjugates in terms of conjugation position and absolute DAR. Unlike most conventional ADC preparations the present invention does not rely entirely on partial or total reduction of the antibody to provide random conjugation sites and relatively uncontrolled generation of DAR species. Rather, the present invention provides one or more predetermined unpaired (or free) cysteine sites by engineering the targeting DLL3 antibody to disrupt one or more of the naturally occurring (i.e., “native”) interchain or intrachain disulfide bridges. Thus, as used herein, the terms “free cysteine” or “unpaired cysteine” may be used interchangeably unless otherwise dictated by context and shall mean any cysteine constituent of an antibody whose native disulfide bridge partner has been substituted, eliminated or otherwise altered to disrupt the naturally occurring disulfide bride under physiological conditions thereby rendering the unpaired cysteine suitable for site-specific conjugation. It will be appreciated that, prior to conjugation, free or unpaired cysteines may be present as a thiol (reduced cysteine), as a capped cysteine (oxidized) or as a non-natural intramolecular disulfide bond (oxidized) with another free cysteine on the same antibody depending on the oxidation state of the system. As discussed in more detail below, mild reduction of this antibody construct will provide thiols available for site specific conjugation.

More specifically the resulting free cysteines may then be selectively reduced using the novel techniques disclosed herein without substantially disrupting intact native disulfide bridges, to provide reactive thiols predominantly at the selected sites. These manufactured thiols are then subject to directed conjugation with the disclosed PBD linker compounds without substantial non-specific conjugation. That is, the engineered constructs and, optionally, the selective reduction techniques disclosed herein largely eliminate non-specific, random conjugation of the PBD payloads. Significantly this provides preparations that are substantially homogeneous in both DAR species distribution and conjugate position on the targeting antibody. As discussed below the elimination of relatively high DAR contaminants can, in and of itself, reduce non-specific toxicity and expand the therapeutic index of the preparation. Moreover, such selectivity allows the payloads to largely be placed in particularly advantageous predetermined positions (such as the terminal region of the light chain constant region) where the payload is somewhat protected until it reaches the tumor but is suitably presented and processed once it reaches the target. Thus, design of the engineered antibody to facilitate specific payload positioning may also be used to reduce the non-specific toxicity of the disclosed preparations.

As discussed below and shown in the Examples, creation of these predetermined free cysteine sites may be achieved using art-recognized molecular engineering techniques to remove, alter or replace one of the constituent cysteine residues of the disulfide bond. Using these techniques one skilled in the art will appreciate that any antibody class or isotype may be engineered to selectively exhibit one or more free cysteine(s) capable of being selectively conjugated in accordance with the instant invention. Moreover, the selected antibody maybe engineered to specifically exhibit 1, 2, 3, 4, 5, 6, 7 or even 8 free cysteines depending on the desired DAR. More preferably the selected antibody will be engineered to contain 2 or 4 free cysteines and even more preferably to contain 2 free cysteines. It will also be appreciated that the free cysteines may be positioned in engineered antibody to facilitate delivery of the selected PBD to the target while reducing non-specific toxicity. In this respect selected embodiments of the invention comprising IgG1 antibodies will position the payload on the C_{H}1 domain and more preferably on the C-terminal end of the domain. In other preferred embodiments the constructs will be engineered to position the payload on the light chain constant region and more preferably at the C-terminal end of the constant region.

Limiting payload positioning to the engineered free cysteines may also be facilitated by selective reduction of the construct using novel stabilization agents a set forth below. “Selective reduction” as used herein will mean exposure of the engineered constructs to reducing conditions that reduce the free cysteines (thereby providing reactive thiols) without substantially disrupting intact native disulfide bonds. In general selective reduction may be effected using any reducing agents, or combinations thereof that provide the desired thiols without disrupting the intact disulfide bonds. In certain preferred embodiments, and as set forth in the Examples below, selective reduction may be effected using a stabilizing agent and mild reducing conditions to prepare the engineered construct for conjugation. As discussed in more detail below compatible stabilizing agents will generally facilitate reduction of the free cysteines and allow the desired conjugation to proceed under less stringent reducing conditions. This allows a substantial majority of the native disulfide bonds to remain intact and markedly reduces the amount of non-specific conjugation thereby limiting unwanted contaminants and potential toxicity. The relatively mild reducing conditions may be attained through the use of a number of systems but preferably comprises the use of thiol containing compounds. One skilled in the art could readily derive compatible reducing systems in view of the instant disclosure.

### II. DLL3 Physiology

It has been found that DLL3 phenotypic determinants are clinically associated with various proliferative disorders, including neoplasia exhibiting neuroendocrine features, and that DLL3 protein and variants or isoforms thereof provide useful tumor markers which may be exploited in the treatment of related diseases. In this regard the present invention provides a number of site-specific antibody drug conjugates comprising an engineered anti-DLL3 antibody targeting agent and PBD payload. As discussed in more detail below and set forth in the appended Examples, the disclosed site-specific anti-DLL3 ADCs are particularly effective at eliminating tumorigenic cells and therefore useful for the treatment and prophylaxis of certain proliferative disorders or the progression or recurrence thereof. In addition, the disclosed site-specific ADC compositions exhibit a relatively high DAR=2 percentage and unexpected stability that may provide for an improved therapeutic index when compared with conventional ADC compositions comprising the same components.

Moreover, it has been found that DLL3 markers or determinants such as cell surface DLL3 protein are therapeutically associated with cancer stem cells (also known as tumor perpetuating cells) and may be effectively exploited to eliminate or silence the same. The ability to selectively reduce or eliminate cancer stem cells through the use of site-specific anti-DLL3 conjugates as disclosed herein is surprising in that such cells are known to generally be resistant to many conventional treatments. That is, the effectiveness of traditional, as well as more recent targeted treatment methods, is often limited by the existence and/or emergence of resistant cancer stem cells that are capable of perpetuating tumor growth even in face of these diverse treatment methods. Further, determinants associated with cancer stem cells often make poor therapeutic targets due to low or inconsistent expression, failure to remain associated with the tumorigenic cell or failure to present at the cell surface. In sharp contrast to the teachings of the prior art, the instantly disclosed site-specific ADCs and methods effectively overcome this inherent resistance and to specifically eliminate, deplete, silence or promote the differentiation of such cancer stem cells thereby negating their ability to sustain or re-induce the underlying tumor growth. As indicated herein the unexpected stability provided by the disclosed, relatively DAR homogeneous preparations

Thus, it is particularly remarkable that DLL3 conjugates such as those disclosed herein may advantageously be used in the treatment and/or prevention of selected proliferative (e.g., neoplastic) disorders or progression or recurrence thereof. It will be appreciated that, while preferred embodiments of the invention will be discussed extensively below, particularly in terms of particular domains, regions or epitopes or in the context of cancer stem cells or tumors comprising neuroendocrine features and their interactions with the disclosed antibody drug conjugates, those skilled in the art will appreciate that the scope of the instant invention is not limited by such exemplary embodiments. Rather, the most expansive embodiments of the present invention and the appended claims are broadly and expressly directed to the disclosed anti-DLL3 site-specific conjugates and their use in the treatment and/or prevention of a variety of DLL3 associated or mediated disorders, including neoplastic or cell proliferative disorders, regardless of any particular mechanism of action or specifically targeted tumor, cellular or molecular component.

In Drosophila, Notch signaling is mediated primarily by one Notch receptor gene and two ligand genes, known as Serrate and Delta (Wharton et al, 1985; Rebay et al., 1991). In humans, there are four known Notch receptors and five DSL (Delta-Serrate LAG2) ligands—two homologs of Serrate, known as Jagged1 and Jagged 2, and three homologs of Delta, termed delta-like ligands or DLL1, DLL3 and DLL4. In general, Notch receptors on the surface of the signal-receiving cell are activated by interactions with ligands expressed on the surface of an opposing, signal-sending cell (termed a trans-interaction). These trans-interactions lead to a sequence of protease mediated cleavages of the Notch receptor. In consequence, the Notch receptor intracellular domain is free to translocate from the membrane to the nucleus, where it partners with the CSL family of transcription factors (RBPJ in humans) and converts them from transcriptional repressors into activators of Notch responsive genes.

Of the human Notch ligands, DLL3 is different in that it seems incapable of activating the Notch receptor via trans-interactions (Ladi et al., 2005). Notch ligands may also interact with Notch receptors in cis (on the same cell) leading to inhibition of the Notch signal, although the exact mechanisms of cis-inhibition remain unclear and may vary depending upon the ligand (for instance, see Klein et al., 1997; Ladi et al., 2005; Glittenberg et al., 2006). Two hypothesized modes of inhibition include modulating Notch signaling at the cell surface by preventing trans-interactions, or by reducing the amount of Notch receptor on the surface of the cell by perturbing the processing of the receptor or by physically causing retention of the receptor in the endoplasmic reticulum or Golgi (Sakamoto et al., 2002; Dunwoodie, 2009). It is clear, however, that stochastic differences in expression of Notch receptors and ligands on neighboring cells can be amplified through both transcriptional and non-transcriptional processes, and subtle balances of cis- and trans-interactions can result in a fine tuning of the Notch mediated delineation of divergent cell fates in neighboring tissues (Sprinzak et al., 2010).

DLL3 is a member of the Delta-like family of Notch DSL ligands. Representative DLL3 protein orthologs include, but are not limited to, human (Accession Nos. NP_058637 and NP_982353), chimpanzee (Accession No. XP_003316395), mouse (Accession No. NP_031892), and rat (Accession No. NP_446118). In humans, the DLL3 gene consists of 8 exons spanning 9.5 kBp located on chromosome 19q13. Alternate splicing within the last exon gives rise to two processed transcripts, one of 2389 bases (Accession No. NM_016941) and one of 2052 bases (Accession No. NM_203486). The former transcript encodes a 618 amino acid protein (Accession No. NP_058637; SEQ ID NO: 1), whereas the latter encodes a 587 amino acid protein (Accession No. NP_982353; SEQ ID NO: 2). These two protein isoforms of DLL3 share overall 100% identity across their extracellular domains and their transmembrane domains, differing only in that the longer isoform contains an extended cytoplasmic tail containing 32 additional residues at the carboxy terminus of the protein. The biological relevance of the isoforms is unclear, although both isoforms can be detected in tumor cells.

The extracellular region of the DLL3 protein, comprises six EGF-like domains, the single DSL domain and the N-terminal domain. Generally, the EGF domains are recognized as occurring at about amino acid residues 216-249 (domain 1), 274-310 (domain 2), 312-351 (domain 3), 353-389 (domain 4), 391-427 (domain 5) and 429-465 (domain 6), with the DSL domain at about amino acid residues 176-215 and the N-terminal domain at about amino acid residues 27-175 of hDLL3 (SEQ ID NOS: 1 and 2). As discussed in more detail herein and shown in the Examples below, each of the EGF-like domains, the DSL domain and the N-terminal domain comprise part of the DLL3 protein as defined by a distinct amino acid sequence. Note that, for the purposes of the instant disclosure the respective EGF-like domains may be termed EGF1 to EGF6 with EGF1 being closest to the N-terminal portion of the protein. In regard to the structural composition of the protein one significant aspect of the instant invention is that the disclosed DLL3 modulators may be generated, fabricated, engineered or selected so as to react with a selected domain, motif or epitope. In certain cases such site-specific modulators may provide enhanced reactivity and/or efficacy depending on their primary mode of action. In particularly preferred embodiments the site-specific anti-DLL3 ADC will bind to the DSL domain and, in even more preferred embodiments, will bind to an epitope comprising G203, R205, P206 (SEQ ID NO: 4) within the DSL domain.

### III. Cell Binding Agents

1. Antibody Structure

As alluded to above, particularly preferred embodiments of the instant invention comprise the disclosed DLL3 conjugates with a cell binding agent in the form of a site-specific antibody, or immunoreactive fragment thereof, that preferentially associates with one or more domains of an isoform of DLL3 protein and, optionally, other DLL family members. In this regard antibodies, and site-specific variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

Note that, for the purposes of the instant application it will be appreciated that the terms “modulator” and “antibody” may be used interchangeably unless otherwise dictated by context. Similarly, the terms “anti-DLL3 conjugate” and “DLL3 conjugate”, or simply “conjugate”, all refer to the site-specific conjugates set forth herein and may be used interchangeably unless otherwise dictated by context.

An “antibody” or “intact antibody” typically refers to a Y-shaped tetrameric protein comprising two heavy (H) and two light (L) polypeptide chains held together by covalent disulfide bonds and non-covalent interactions. Human light chains comprise a variable domain (V_{L}) and a constant domain (C_{L}) wherein the constant domain may be readily classified as kappa or lambda based on amino acid sequence and gene loci. Each heavy chain comprises one variable domain (V_{H}) and a constant region, which in the case of IgG, IgA, and IgD, comprises three domains termed C_{H}1, C_{H}2, and C_{H}3 (IgM and IgE have a fourth domain, C_{H}4). In IgG, IgA, and IgD classes the C_{H}1 and C_{H}2 domains are separated by a flexible hinge region, which is a proline and cysteine rich segment of variable length (generally from about 10 to about 60 amino acids in IgG). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

There are two types of native disulfide bridges or bonds in immunoglobulin molecules: interchain and intrachain disulfide bonds. The location and number of interchain disulfide bonds vary according to the immunoglobulin class and species. While the invention is not limited to any particular class or subclass of antibody, the IgG1 immunoglobulin shall be used for illustrative purposes only. Interchain disulfide bonds are located on the surface of the immunoglobulin, are accessible to solvent and are usually relatively easily reduced. In the human IgG1 isotype there are four interchain disulfide bonds, one from each heavy chain to the light chain and two between the heavy chains. The interchain disulfide bonds are not required for chain association. The cysteine rich IgG1 hinge region of the heavy chain has generally been held to consist of three parts: an upper hinge (Ser-Cys-Asp-Lys-Thr-His-Thr), a core hinge (Cys-Pro-Pro-Cys), and a lower hinge (Pro-Ala-Glu-Leu-Leu-Gly-Gly). Those skilled in the art will appreciate that that the IgG1 hinge region contain the cysteines in the heavy chain that comprise the interchain disulfide bonds (two heavy/heavy, two heavy/light), which provide structural flexibility that facilitates Fab movements.

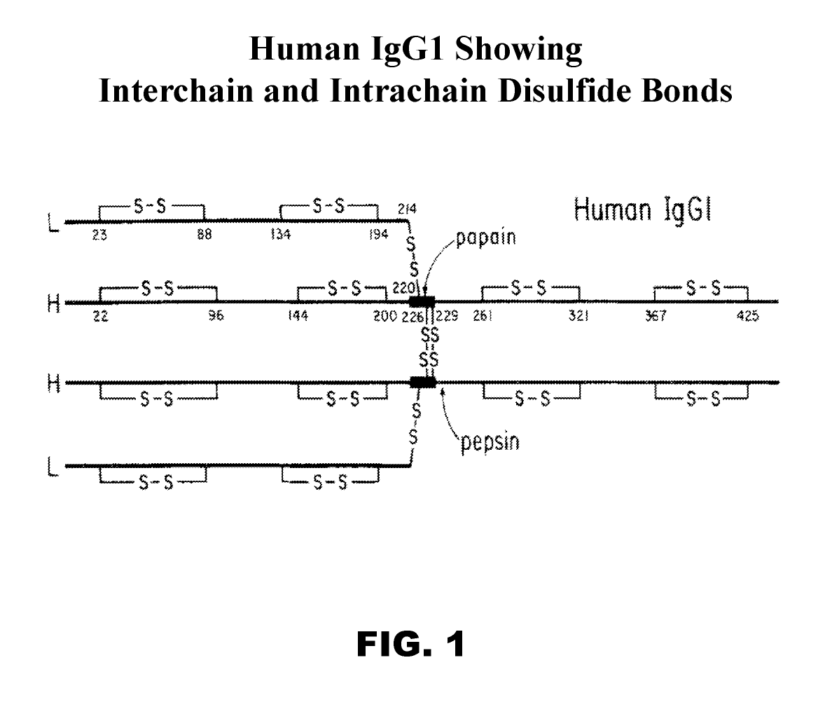

The interchain disulfide bond between the light and heavy chain of IgG1 are formed between C214 of the kappa or lambda light chain and C220 in the upper hinge region of the heavy chain (FIG. 1). The interchain disulfide bonds between the heavy chains are at positions C226 and C229. (all numbered per the EU index according to Kabat, et al., infra.)

As used herein the term “antibody” may be construed broadly and includes polyclonal antibodies, multiclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a DLL3 determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

In selected embodiments and as discussed in more detail below, the C_{L} domain may comprise a kappa C_{L} domain exhibiting a free cysteine. In other embodiments the source antibody may comprise a lambda C_{L} domain exhibiting a free cysteine. As the sequences of all human IgG C_{L} domains are well known, one skilled in the art may easily analyze both lambda and kappa sequences in accordance with the instant disclosure and employ the same to provide compatible antibody constructs. Similarly, for the purposes of explanation and demonstration the following discussion and appended Examples will primarily feature the IgG1 type antibodies. As with the light chain constant region, heavy chain constant domain sequences from different isotypes (IgM, IgD, IgE, IgA) and subclasses (IgG1, IgG2, IgG3, IgG4, IgA1, IgA2) are well known and characterized. Accordingly, one skilled in the art may readily exploit anti-DLL3 antibodies comprising any isotype or subclass and conjugate each with the disclosed PBDs as taught herein to provide the site-specific antibody drug conjugates of the present invention.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). V_{H} and V_{L} domains comprise three regions of extreme variability, which are termed hypervariable regions, or more commonly, complementarity-determining regions (CDRs), framed and separated by four less variable regions known as framework regions (FRs). The non-covalent association between the V_{H} and the V_{L} region forms the Fv fragment (for “fragment variable”) which contains one of the two antigen-binding sites of the antibody. ScFv fragments (for single chain fragment variable), which can be obtained by genetic engineering, associates in a single polypeptide chain, the V_{H} and the V_{L} region of an antibody, separated by a peptide linker.

As used herein, the assignment of amino acids to each domain, framework region and CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co. unless otherwise noted. Amino acid residues which comprise CDRs as defined by Kabat, Chothia and MacCallum as obtained from the Abysis website database (infra.) are set out below

TABLE 1

| | Kabat | Chothia | MacCallum |
|---|---|---|---|
| V_{H} CDR1 | 31-35 | 26-32 | 30-35 |
| V_{H} CDR2 | 50-65 | 52-56 | 47-58 |
| V_{H} CDR3 | 95-102 | 95-102 | 93-101 |
| V_{L} CDR1 | 24-34 | 24-34 | 30-36 |
| V_{L} CDR2 | 50-56 | 50-56 | 46-55 |
| V_{L} CDR3 | 89-97 | 89-97 | 89-96 |

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N. J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat.

For heavy chain constant region amino acid positions discussed in the invention, numbering is according to the Eu index first described in Edelman et al., 1969, Proc, Natl. Acad. Sci. USA 63(1): 78-85 describing the amino acid sequence of myeloma protein Eu, which reportedly was the first human IgG1 sequenced. The Eu index of Edelman is also set forth in Kabat et al., 1991 (supra.). Thus, the terms “EU index as set forth in Kabat” or “EU index of Kabat” or “EU index according to Kabat” in the context of the heavy chain refers to the residue numbering system based on the human IgG1 Eu antibody of Edelman et al. as set forth in Kabat et al., 1991 (supra.). The numbering system used for the light chain constant region amino acid sequence is similarly set forth in Kabat et al., 1991.

Exemplary kappa C_{L} and IgG1 heavy chain constant region amino acid sequences compatible with the instant invention are set forth as SEQ ID NOS: 5 and 6 in the appended sequence listing. Similarly, an exemplary lambda C_{L} light chain constant region is set forth as SEQ ID NO: 11 in the appended sequence listing. Those of skill in the art will appreciate that such light chain constant region sequences, engineered as disclosed herein to provide unpaired cysteines (e.g., see SEQ ID NOS: 7-10, 12 and 13), may be joined with the disclosed heavy and light chain variable regions using standard molecular biology techniques to provide full-length antibodies (see SEQ ID NOS: 14-19) that may be incorporated in the DLL3 conjugates of the instant invention.

The site-specific antibodies or immunoglobulins of the invention may comprise, or be derived from, any antibody that specifically recognizes or associates with any DLL3 determinant. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In certain preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a DLL3 protein, or any of its splice variants, isoforms or family members, or specific domains, regions or epitopes thereof. An “antigen”, “immunogenic determinant”, “antigenic determinant” or “immunogen” means any protein (including DLL3) or any fragment, region, domain or epitope thereof that can stimulate an immune response when introduced into an immunocompetent animal and is recognized by antibodies produced from the immune response of the animal. The presence or absence of the determinants contemplated herein may be used to identify a cell, cell subpopulation or tissue (e.g., tumors, tumorigenic cells or CSCs).

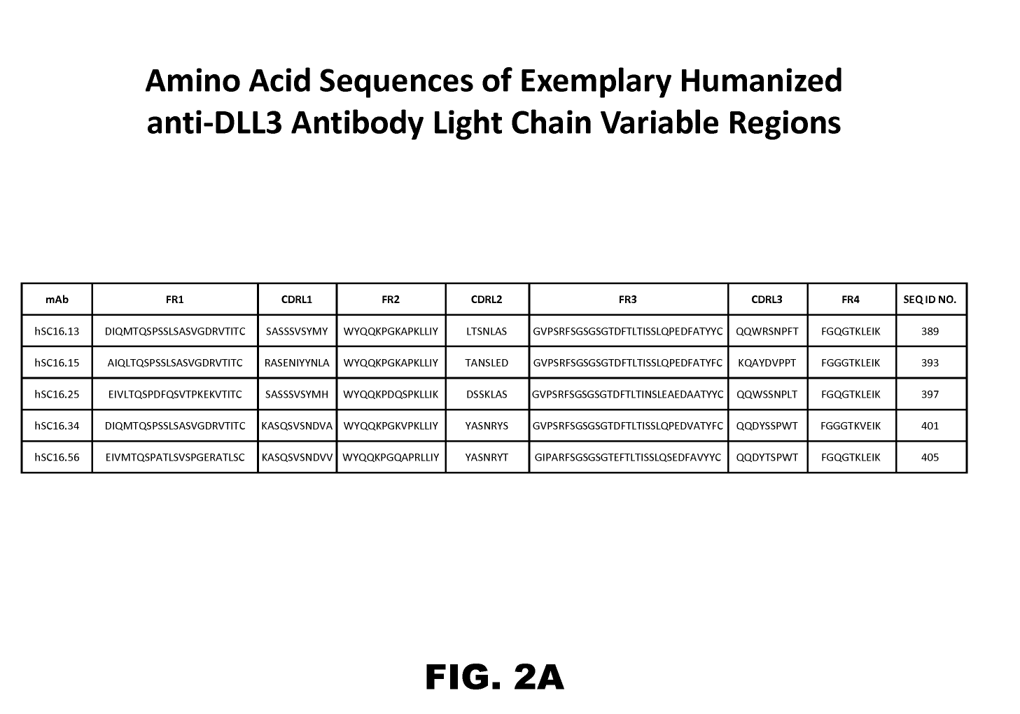

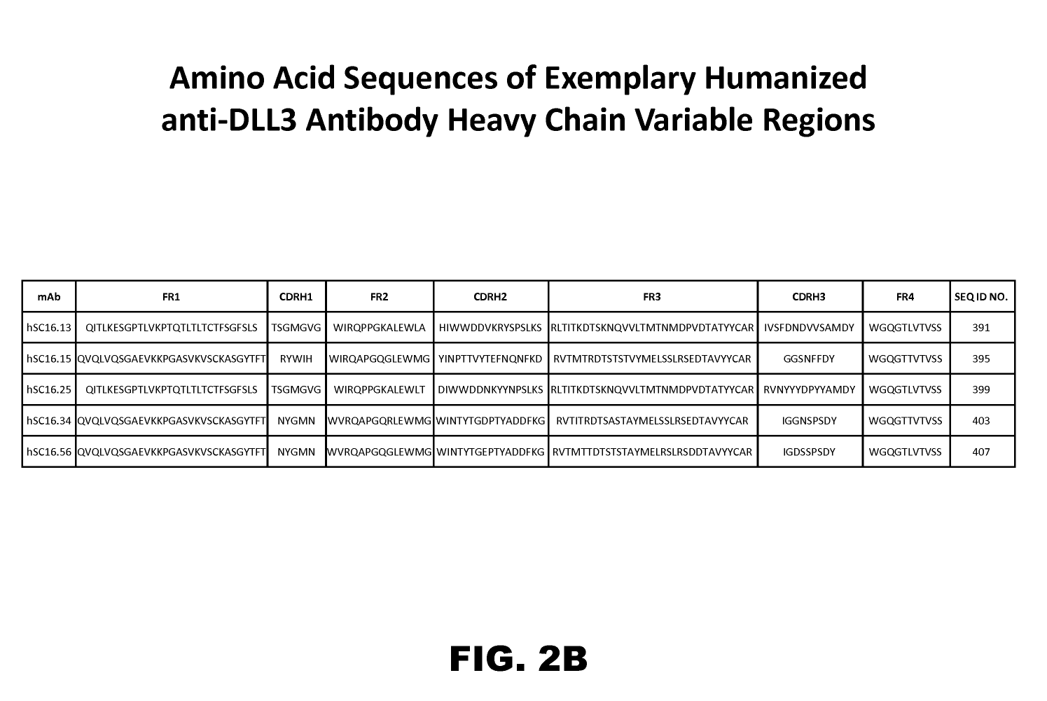

As set forth below in the Examples, selected embodiments of the invention comprise murine antibodies that immunospecifically bind to DLL3, which can be considered “source” antibodies. In other embodiments, antibodies contemplated by the invention may be derived from such “source” antibodies through optional modification of the constant region (i.e., to provide site-specific antibodies) or the epitope-binding amino acid sequences of the source antibody. In one embodiment an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or the entire variable region) are combined with or incorporated into an acceptor antibody sequence to provide the derivative antibody (e.g. chimeric, CDR grafted or humanized antibodies). These “derived” (e.g. humanized or CDR-grafted) antibodies can be generated using standard molecular biology techniques for various reasons such as, for example, to improve affinity for the determinant; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification. Of course, as discussed extensively herein these derived antibodies may be further engineered to provide the desired site-specific antibodies comprising one or more free cysteines. In the context of the instant invention it will be appreciated that any of the disclosed light and heavy chain CDRs derived from the murine variable region amino acid sequences set forth in the appended sequence listing may be combined with acceptor antibodies or rearranged to provide optimized anti-human DLL3 (e.g. humanized or chimeric anti-hDLL3) site-specific antibodies in accordance with the instant teachings. That is, one or more of the CDRs derived or obtained from the contiguous light chain variable region amino acid sequences set forth in the appended sequence listing (together SEQ ID NOS: 21-387, odd numbers) may be incorporated in a site-specific construct and, in particularly preferred embodiments, in a CDR grafted or humanized site-specific antibody that immunospecifically associates with one or more DLL3 isoforms. Examples of “derived” light and heavy chain variable region amino acid sequences of such humanized modulators are also set forth in FIGS. 2A and 2B (SEQ ID NOS: 389-407, odd numbers).

In FIGS. 2A and 2B the annotated CDRs and framework sequences are defined as per Kabat using a proprietary Abysis database. However, as discussed herein one skilled in the art could readily define, identify, derive and/or enumerate the CDRs as defined by Kabat et al., Chothia et al. or MacCallum et al. for each respective heavy and light chain sequence set forth in the appended sequence listing. Accordingly, each of the subject CDRs and antibodies comprising CDRs defined by all such nomenclature are expressly included within the scope of the instant invention. More broadly, the terms “variable region CDR amino acid residue” or more simply “CDR” includes amino acids in a CDR as identified using any sequence or structure based method as set forth above. Within this context Kabat CDRs for the exemplary humanized antibodies in FIGS. 2A and 2B are provided in the appended sequence listing as SEQ ID NOS: 408-437.

Another aspect of the invention comprises ADCs incorporating antibodies obtained or derived from SC16.3, SC16.4, SC16.5, SC16.7, SC16.8, SC16.10, SC16.11, SC16.13, SC16.15, SC16.18, SC16.19, SC16.20, SC16.21, SC16.22, SC16.23, SC16.25, SC16.26, SC16.29, SC16.30, SC16.31, SC16.34, SC16.35, SC16.36, SC16.38, SC16.41, SC16.42, SC16.45, SC16.47, SC16.49, SC16.50, SC16.52, SC16.55, SC16.56, SC16.57, SC16.58, SC16.61, SC16.62, SC16.63, SC16.65, SC16.67, SC16.68, SC16.72, SC16.73, SC16.78, SC16.79, SC16.80, SC16.81, SC16.84, SC16.88, SC16.101, SC16.103, SC16.104, SC16.105, SC16.106, SC16.107, SC16.108, SC16.109, SC16.110, SC16.111, SC16.113, SC16.114, SC16.115, SC16.116, SC16.117, SC16.118, SC16.120, SC16.121, SC16.122, SC16.123, SC16.124, SC16.125, SC16.126, SC16.129, SC16.130, SC16.131, SC16.132, SC16.133, SC16.134, SC16.135, SC16.136, SC16.137, SC16.138, SC16.139, SC16.140, SC16.141, SC16.142, SC16.143, SC16.144, SC16.147, SC16.148, SC16.149 and SC16.150; or any of the above-identified antibodies, or chimeric or humanized versions thereof. In other embodiments the ADCs of the invention will comprise a DLL3 antibody having one or more CDRs, for example, one, two, three, four, five, or six CDRs, from any of the aforementioned modulators. The annotated sequence listing provides the individual SEQ ID NOS for the heavy and light chain variable regions for each of the aforementioned anti-DLL3 antibodies.

2. Site-Specific Antibodies

Based on the instant disclosure one skilled in the art could readily fabricate engineered constructs as described herein. As used herein, “engineered antibody” “engineered construct” or “site-specific antibody” means an antibody, or immunoreactive fragment thereof, wherein at least one amino acid in either the heavy or light chain is deleted, altered or substituted (preferably with another amino acid) to provide at least one free cysteine. Similarly, an “engineered conjugate” or “site-specific conjugate” shall be held to mean an antibody drug conjugate comprising an engineered antibody and at least one PBD conjugated to the unpaired cysteine(s). In certain embodiments the unpaired cysteine residue will comprise an unpaired intrachain residue. In other preferred embodiments the free cysteine residue will comprise an unpaired interchain cysteine residue. The engineered antibody can be of various isotypes, for example, IgG, IgE, IgA or IgD; and within those classes the antibody can be of various subclasses, for example, IgG1, IgG2, IgG3 or IgG4. With regard to such IgG constructs the light chain of the antibody can comprise either a kappa or lambda isotype each incorporating a C214 that, in preferred embodiments, may be unpaired due to a lack of a C220 residue in the IgG1 heavy chain.

In one embodiment the engineered antibody comprises at least one amino acid deletion or substitution of an intrachain or interchain cysteine residue. As used herein “interchain cysteine residue” means a cysteine residue that is involved in a native disulfide bond either between the light and heavy chain of an antibody or between the two heavy chains of an antibody while an intrachain cysteine residue is one naturally paired with another cysteine in the same heavy or light chain. In one embodiment the deleted or substituted interchain cysteine residue is in involved in the formation of a disulfide bond between the light and heavy chain. In another embodiment the deleted or substituted cysteine residue is involved in a disulfide bond between the two heavy chains.

In a typical embodiment, due to the complementary structure of an antibody, in which the light chain is paired with the V_{H} and C_{H}1 domains of the heavy chain and wherein the C_{H}2 and C_{H}3 domains of one heavy chain are paired with the C_{H}2 and C_{H}3 domains of the complementary heavy chain, a mutation or deletion of a single cysteine in either the light chain or in the heavy chain would result in two unpaired cysteine residues in the engineered antibody.

In some embodiments an interchain cysteine residue is deleted. In other embodiments an interchain cysteine is substituted for another amino acid (e.g., a naturally occurring amino acid). For example, the amino acid substitution can result in the replacement of an interchain cysteine with a neutral (e.g. serine, threonine or glycine) or hydrophilic (e.g. methionine, alanine, valine, leucine or isoleucine) residue. In one particularly preferred embodiment an interchain cysteine is replaced with a serine.

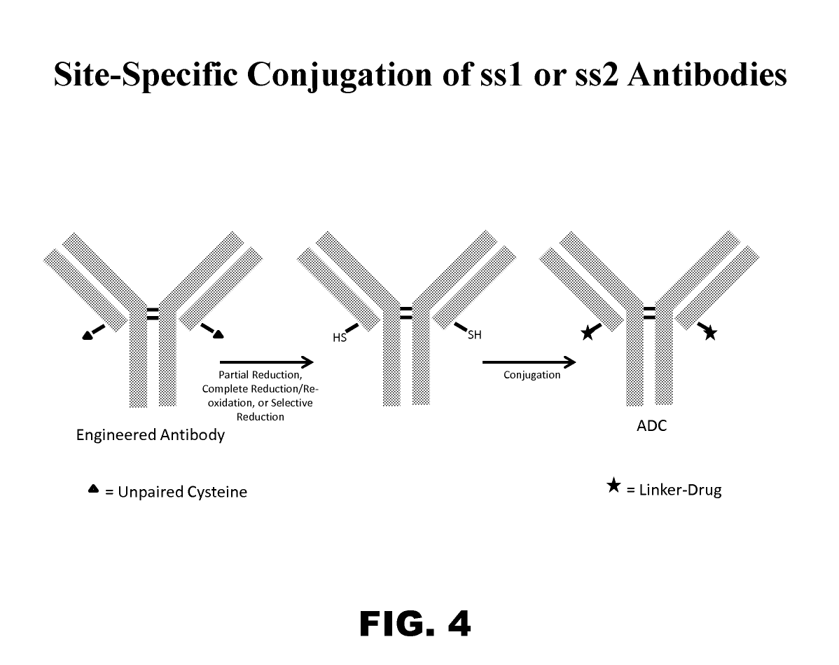

In some embodiments contemplated by the invention the deleted or substituted cysteine residue is on the light chain (either kappa or lambda) thereby leaving a free cysteine on the heavy chain. In other embodiments the deleted or substituted cysteine residue is on the heavy chain leaving the free cysteine on the light chain constant region. FIG. 1 depicts the cysteines involved in the interchain disulfide bonds in an exemplary IgG1/kappa antibody. As previously indicated in each case the amino acid residues of the constant regions are numbered based on the EU index according to Kabat. As shown in FIG. 4, deletion or substitution of a single cysteine in either the light or heavy chain of an intact antibody results in an engineered antibody having two unpaired cysteine residues.

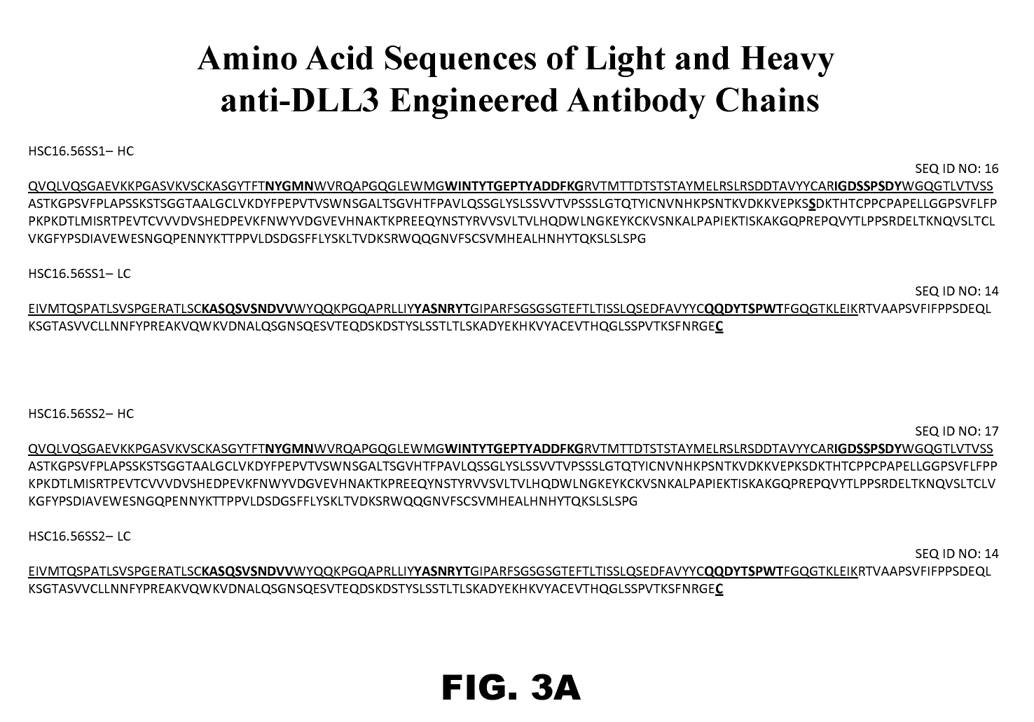

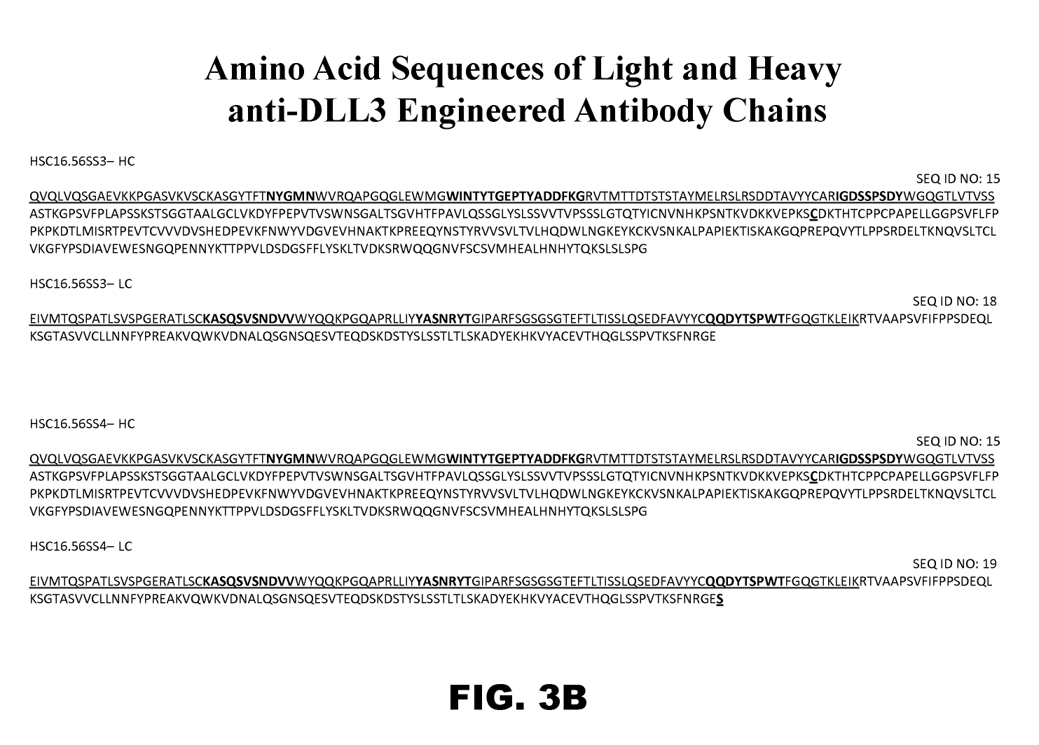

In one particularly preferred embodiment the cysteine at position 214 (C214) of the IgG light chain (kappa or lambda) is deleted or substituted. In another preferred embodiment the cysteine at position 220 (C220) on the IgG heavy chain is deleted or substituted. In further embodiments the cysteine at position 226 or position 229 on the heavy chain is deleted or substituted. In one embodiment C220 on the heavy chain is substituted with serine (C220S) to provide the desired free cysteine in the light chain. In another embodiment C214 in the light chain is substituted with serine (C214S) to provide the desired free cysteine in the heavy chain. Such site-engineered constructs provided as per Example 4 are shown in FIGS. 3A and 3B using the exemplary anti-DLL3 antibody SC16.56. A summary of these preferred constructs is shown in Table 2 immediately below where all numbering is according to the EU index as set forth in Kabat and WT stands for “wild-type” or native constant region sequences without alterations. Note that, while the referenced sequences are kappa light chains, exemplary lambda light chains comprising C214 may also be used as set forth herein. Also, as used herein delta (Δ) shall designate the deletion of an amino acid residue (e.g., C214Δ indicates that the cysteine at position 214 has been deleted).

TABLE 2

| | Antibody | | Const. Reg. |
|---|---|---|---|
| Designation | Component | Alteration | SEQ ID NO: |
| ss1 | Heavy Chain | C220S | 7 |
| | Light Chain | WT | 5 |
| ss2 | Heavy Chain | C220Δ | 8 |
| | Light Chain | WT | 5 |
| ss3 | Heavy Chain | WT | 6 |
| | Light Chain | C214Δ | 9 |
| ss4 | Heavy Chain | WT | 6 |
| | Light Chain | C214S | 10 |

The strategy for generating antibody-drug conjugates with defined sites and stoichiometries of drug loading, as disclosed herein, is broadly applicable to other antibodies as it primarily involves engineering of the conserved constant domains of the antibody. As the amino acid sequences and native disulfide bridges of each class and subclass of antibody are well documented, one skilled in the art could readily fabricate engineered constructs of various antibodies without undue experimentation and, accordingly, such constructs are expressly contemplated as being within the scope of the instant invention.

3. Antibody Generation

a. Polyclonal Antibodies

The production of polyclonal antibodies in various host animals, including rabbits, mice, rats, etc. is well known in the art. In some embodiments, polyclonal anti-DLL3 antibody-containing serum is obtained by bleeding or sacrificing the animal. The serum may be used for research purposes in the form obtained from the animal or, in the alternative, the anti-DLL3 antibodies may be partially or fully purified to provide immunoglobulin fractions or homogeneous antibody preparations.

Briefly the selected animal is immunized with a DLL3 immunogen (e.g., soluble DLL3 or sDLL3) which may, for example, comprise selected isoforms, domains and/or peptides, or live cells or cell preparations expressing DLL3 or immunoreactive fragments thereof. Art known adjuvants that may be used to increase the immunological response, depending on the inoculated species include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants may protect the antigen from rapid dispersal by sequestering it in a local deposit, or they may contain substances that stimulate the host to secrete factors that are chemotactic for macrophages and other components of the immune system. Preferably the immunization schedule will involve two or more administrations of the selected immunogen spread out over a predetermined period of time.

The amino acid sequence of a DLL3 protein as shown in FIG. 1 can be analyzed to select specific regions of the DLL3 protein for generating antibodies. For example, hydrophobicity and hydrophilicity analyses of a DLL3 amino acid sequence are used to identify hydrophilic regions in the DLL3 structure. Regions of a DLL3 protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Average Flexibility profiles can be generated using the method of Bhaskaran R., Ponnuswamy P. K., 1988, Int. J. Pept. Protein Res. 32:242-255. Beta-turn profiles can be generated using the method of Deleage, G., Roux B., 1987, Protein Engineering 1:289-294. Thus, each DLL3 region, domain or motif identified by any of these programs or methods is within the scope of the present invention and may be isolated or engineered to provide immunogens giving rise to modulators comprising desired properties. Preferred methods for the generation of DLL3 antibodies are further illustrated by way of the Examples provided herein. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carbodiimide reagents are used; in other instances linking reagents are effective. Administration of a DLL3 immunogen is often conducted by injection over a suitable time period and with use of a suitable adjuvant, as is understood in the art. During the immunization schedule, titers of antibodies can be taken as described in the Examples below to determine adequacy of antibody formation.

b. Monoclonal Antibodies

In addition, the invention contemplates use of monoclonal antibodies. As known in the art, the term “monoclonal antibody” (or mAb) refers to an antibody obtained from a population of substantially homogeneous antibodies, i.e., the individual antibodies comprising the population are identical except for possible mutations (e.g., naturally occurring mutations) that may be present in minor amounts. In certain embodiments, such a monoclonal antibody includes an antibody comprising a polypeptide sequence that binds or associates with an antigen wherein the antigen-binding polypeptide sequence was obtained by a process that includes the selection of a single target binding polypeptide sequence from a plurality of polypeptide sequences.

More generally, and as set forth in the Examples herein, monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and art-recognized biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N. Y., 1981) each of which is incorporated herein in its entirety by reference. It should be understood that a selected binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also an antibody of this invention. Murine monoclonal antibodies compatible with the instant invention are provided as set forth in Example 1 below.

c. Chimeric and Humanized Antibodies

In another embodiment, the antibodies of the invention may comprise chimeric antibodies derived from covalently joined protein segments from at least two different species or class of antibodies. The term “chimeric” antibodies is directed to constructs in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass, as well as fragments of such antibodies (U.S. Pat. No. 4,816,567; Morrison et al., 1984, PMID: 6436822).

In one embodiment, a chimeric antibody may comprise murine V_{H} and V_{L} amino acid sequences and constant regions derived from human sources, for example, humanized antibodies as described below. In some embodiments, the antibodies can be “CDR-grafted”, where the antibody comprises one or more CDRs from a particular species or belonging to a particular antibody class or subclass, while the remainder of the antibody chain(s) is/are identical with or homologous to a corresponding sequence in antibodies derived from another species or belonging to another antibody class or subclass. For use in humans, selected rodent CDRs, e.g., mouse CDRs may be grafted into a human antibody, replacing one or more of the naturally occurring CDRs of the human antibody. These constructs generally have the advantages of providing full strength antibody functions, e.g., complement dependent cytotoxicity (CDC) and antibody-dependent cell-mediated cytotoxicity (ADCC) while reducing unwanted immune responses to the antibody by the subject.

Similar to the CDR-grafted antibody is a “humanized” antibody. As used herein, “humanized” forms of non-human (e.g., murine) antibodies are chimeric antibodies that comprise amino acids sequences derived from one or more non-human immunoglobulins. In one embodiment, a humanized antibody is a human immunoglobulin (recipient or acceptor antibody) in which residues from one or more CDRs of the recipient are replaced by residues from one or more CDRs of a non-human species (donor antibody) such as mouse, rat, rabbit, or non-human primate. In certain preferred embodiments, residues in one or more FRs in the variable domain of the human immunoglobulin are replaced by corresponding non-human residues from the donor antibody to help maintain the appropriate three-dimensional configuration of the grafted CDR(s) and thereby improve affinity. This can be referred to as the introduction of “back mutations”. Furthermore, humanized antibodies may comprise residues that are not found in the recipient antibody or in the donor antibody to, for example, further refine antibody performance. Humanized anti-DLL3 antibodies compatible with the instant invention are provided in Example 3 below with resulting humanized light and heavy chain amino acid sequences shown in FIGS. 2A and 2B. FIGS. 3A and 3B show site-specific exemplary humanized anti-DLL3 antibody heavy and light chain annotated amino acid sequences.

Various sources can be used to determine which human sequences to use in the humanized antibodies. Such sources include human germline sequences that are disclosed, for example, in Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638; the V-BASE directory (VBASE2—Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK); or consensus human FRs described, for example, in U.S. Pat. No. 6,300,064.

CDR grafting and humanized antibodies are described, for example, in U.S. Pat. Nos. 6,180,370 and 5,693,762. For further details, see, e.g., Jones et al., 1986, PMID: 3713831); and U.S. Pat. Nos. 6,982,321 and 7,087,409.

Another method is termed “humaneering” which is described, for example, in U.S.P.N. 2005/0008625. In another embodiment a non-human antibody may be modified by specific deletion of human T-cell epitopes or “deimmunization” by the methods disclosed in WO 98/52976 and WO 00/34317.

As discussed above in selected embodiments at least 60%, 65%, 70%, 75%, or 80% of the humanized or CDR grafted antibody heavy or light chain variable region amino acid residues will correspond to those of the recipient human sequences. In other embodiments at least 83%, 85%, 87% or 90% of the humanized antibody variable region residues will correspond to those of the recipient human sequences. In a further preferred embodiment, greater than 95% of each of the humanized antibody variable regions will correspond to those of the recipient human sequences.

The sequence identity or homology of the humanized antibody variable region to the human acceptor variable region may be determined as previously discussed and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

d. Human Antibodies

In another embodiment, the antibodies may comprise fully human antibodies. The term “human antibody” refers to an antibody which possesses an amino acid sequence that corresponds to that of an antibody produced by a human and/or has been made using any of the techniques for making human antibodies.

Human antibodies can be produced using various techniques known in the art. One technique is phage display in which a library of (preferably human) antibodies is synthesized on phages, the library is screened with the antigen of interest or an antibody-binding portion thereof, and the phage that binds the antigen is isolated, from which one may obtain the immunoreactive fragments. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; and Barbas et al., Proc. Natl. Acad. Sci. USA 88:7978-7982 (1991)).

In one embodiment, recombinant human antibodies may be isolated by screening a recombinant combinatorial antibody library prepared as above. In one embodiment, the library is a scFv phage display library, generated using human V_{L} and V_{H} cDNAs prepared from mRNA isolated from B-cells.

The antibodies produced by naive libraries (either natural or synthetic) can be of moderate affinity (Kₐ of about 10⁶ to 10⁷ M⁻¹), but affinity maturation can also be mimicked in vitro by constructing and reselecting from secondary libraries as described in the art. For example, mutation can be introduced at random in vitro by using error-prone polymerase (reported in Leung et al., Technique, 1: 11-15 (1989)). Additionally, affinity maturation can be performed by randomly mutating one or more CDRs, e.g. using PCR with primers carrying random sequence spanning the CDR of interest, in selected individual Fv clones and screening for higher-affinity clones. WO 9607754 described a method for inducing mutagenesis in a CDR of an immunoglobulin light chain to create a library of light chain genes. Another effective approach is to recombine the V_{H} or V_{L} domains selected by phage display with repertoires of naturally occurring V domain variants obtained from unimmunized donors and to screen for higher affinity in several rounds of chain reshuffling as described in Marks et al., Biotechnol., 10: 779-783 (1992). This technique allows the production of antibodies and antibody fragments with a dissociation constant K_{H} (k_{off}/kₒₙ) of about 10⁻⁹ M or less.

In other embodiments, similar procedures may be employed using libraries comprising eukaryotic cells (e.g., yeast) that express binding pairs on their surface. See, for example, U.S. Pat. No. 7,700,302 and U.S. Ser. No. 12/404,059. In one embodiment, the human antibody is selected from a phage library, where that phage library expresses human antibodies (Vaughan et al. Nature Biotechnology 14:309-314 (1996): Sheets et al. Proc. Natl. Acad. Sci. USA 95:6157-6162 (1998). In other embodiments, human binding pairs may be isolated from combinatorial antibody libraries generated in eukaryotic cells such as yeast. See e.g., U.S. Pat. No. 7,700,302. Such techniques advantageously allow for the screening of large numbers of candidate modulators and provide for relatively easy manipulation of candidate sequences (e.g., by affinity maturation or recombinant shuffling).

Human antibodies can also be made by introducing human immunoglobulin loci into transgenic animals, e.g., mice in which the endogenous immunoglobulin genes have been partially or completely inactivated and human immunoglobulin genes have been introduced. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and U.S. Pat. Nos. 6,075,181 and 6,150,584 regarding XenoMouse® technology; and Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., J. Immunol, 147 (1):86-95 (1991); and U.S. Pat. No. 5,750,373.

4. Recombinant Production of Antibodies

The site-specific antibodies and fragments thereof may be produced or modified using genetic material obtained from antibody producing cells and recombinant technology (see, for example, Berger and Kimmel, Guide to Molecular Cloning Techniques, Methods in Enzymology vol. 152 Academic Press, Inc., San Diego, Calif.; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc. (supplemented through 2006); and U.S. Pat. No. 7,709,611).

More particularly, another aspect of the invention pertains to engineered nucleic acid molecules that encode the site-specific antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA or RNA and may or may not contain intronic sequences. More generally the term “nucleic acid”, as used herein, includes genomic DNA, cDNA, RNA and artificial variants thereof (e.g., peptide nucleic acids), whether single-stranded or double-stranded. In a preferred embodiment, the nucleic acid is a cDNA molecule.

Nucleic acids of the invention can be obtained and manipulated using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques (e.g., see Example 1). For antibodies obtained from an immunoglobulin gene library (e.g., using phage display techniques), nucleic acid encoding the antibody can be recovered from the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H}1, C_{H}2 and C_{H}3) which may or may not be engineered as described herein. The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. As discussed in more detail below an exemplary IgG1 constant region that is compatible with the teachings herein is set forth as SEQ ID NO: 6 in the appended sequence listing with compatible engineered IgG1 constant regions set forth in SEQ ID NOS: 7 and 8. For a Fab fragment heavy chain gene, the VH-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region. In this respect an exemplary compatible kappa light chain constant region is set forth as SEQ ID NO: 5 in the appended sequence listing while a compatible lambda light chain constant region is set forth in SEQ ID NO: 11. Compatible engineered versions of the kappa and lambda light chain regions are shown in SEQ ID NOS: 9-10 and 12-13 respectively.

The instant invention also provides vectors comprising such nucleic acids described above, which may be operably linked to a promoter (see, e.g., WO 86/05807; WO 89/01036; and U.S. Pat. No. 5,122,464); and other transcriptional regulatory and processing control elements of the eukaryotic secretory pathway. The invention also provides host cells harboring those vectors and host-expression systems.

As used herein, the term “host-expression system” includes any kind of cellular system which can be engineered to generate either the nucleic acids or the polypeptides and antibodies of the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO—S, HEK-293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity).

For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under certain conditions. The GS system is discussed in whole or part in connection with U.S. Pat. Nos. 5,591,639 and 5,879,936. Another preferred expression system for the development of stable cell lines is the Freedom™ CHO—S Kit (Life Technologies).

Once an antibody of the invention has been produced by recombinant expression or any other of the disclosed techniques, it may be purified or isolated by methods known in the art, meaning that it is identified and separated and/or recovered from its natural environment and separated from contaminants that would interfere with conjugation or diagnostic or therapeutic uses for the antibody. Isolated antibodies include antibodies in situ within recombinant cells.

These isolated preparations may be purified using various art recognized techniques, such as, for example, ion exchange and size exclusion chromatography, dialysis, diafiltration, and affinity chromatography, particularly Protein A or Protein G affinity chromatography.

5. Antibody Fragments and Derivatives

a. Fragments

Regardless of which form of site-specific antibody (e.g. chimeric, humanized, etc.) is selected to practice the invention it will be appreciated that immunoreactive fragments of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” refers to a polypeptide fragment of an immunoglobulin or antibody comprising at least one free cysteine that immunospecifically binds or reacts with a selected antigen or immunogenic determinant thereof or competes with the intact antibody from which the fragments were derived for specific antigen binding.

Exemplary site-specific fragments include: V_{L}, V_{H}, scFv, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency).

In other embodiments, a site-specific antibody fragment is one that comprises the Fc region and that retains at least one of the biological functions normally associated with the Fc region when present in an intact antibody, such as FcRn binding, antibody half-life modulation, ADCC function and complement binding. In one embodiment, a site-specific antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

b. Multivalent Antibodies

In one embodiment, the site-specific conjugates of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105. In each case at least one of the binding sites will comprise an epitope, motif or domain associated with a DLL3 isoform.

In one embodiment, the modulators are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As alluded to above, multivalent antibodies may immunospecifically bind to different epitopes of the desired target molecule or may immunospecifically bind to both the target molecule as well as a heterologous epitope, such as a heterologous polypeptide or solid support material. While preferred embodiments of the anti-DLL3 antibodies only bind two antigens (i.e. bispecific antibodies), antibodies with additional specificities such as trispecific antibodies are also encompassed by the instant invention. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

In yet other embodiments, antibody variable domains with the desired binding specificities (antibody-antigen combining sites) are fused to immunoglobulin constant domain sequences, such as an immunoglobulin heavy chain constant domain comprising at least part of the hinge, C_{H}2, and/or C_{H}3 regions, using methods well known to those of ordinary skill in the art.

c. Fc Region Modifications

In addition to the various modifications, substitutions, additions or deletions to the variable or binding region of the disclosed site-specific conjugates set forth above, including those generating a free cysteine, those skilled in the art will appreciate that selected embodiments of the present invention may also comprise substitutions or modifications of the constant region (i.e. the Fc region). More particularly, it is contemplated that the DLL3 antibodies of the invention may contain inter alia one or more additional amino acid residue substitutions, mutations and/or modifications which result in a compound with preferred characteristics including, but not limited to: altered pharmacokinetics, increased serum half life, increase binding affinity, reduced immunogenicity, increased production, altered Fc ligand binding to an Fc receptor (FcR), enhanced or reduced “ADCC” (antibody-dependent cell mediated cytotoxicity) or “CDC” (complement-dependent cytotoxicity) activity, altered glycosylation and/or disulfide bonds and modified binding specificity. In this regard it will be appreciated that these Fc variants may advantageously be used to enhance the effective anti-neoplastic properties of the disclosed modulators.

To this end certain embodiments of the invention may comprise substitutions or modifications of the Fc region beyond those required to generate a free cysteine, for example the addition of one or more amino acid residue, substitutions, mutations and/or modifications to produce a compound with enhanced or preferred Fc effector functions. For example, changes in amino acid residues involved in the interaction between the Fc domain and an Fc receptor (e.g., FcγRI, FcγRIIA and B, FcγRIII and FcRn) may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas et al., J. Lab. Clin. Med. 126:330-41 (1995) each of which is incorporated herein by reference).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311. With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibodies and/or reduces the concentration of the antibodies to be administered. Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001).

In other embodiments, Fc alterations may lead to enhanced or reduced ADCC or CDC activity. As in known in the art, CDC refers to the lysing of a target cell in the presence of complement, and ADCC refers to a form of cytotoxicity in which secreted Ig bound onto FcRs present on certain cytotoxic cells (e.g., Natural Killer cells, neutrophils, and macrophages) enables these cytotoxic effector cells to bind specifically to an antigen-bearing target cell and subsequently kill the target cell with cytotoxins. In the context of the instant invention antibody variants are provided with “altered” FcR binding affinity, which is either enhanced or diminished binding as compared to a parent or unmodified antibody or to an antibody comprising a native sequence FcR. Such variants which display decreased binding may possess little or no appreciable binding, e.g., 0-20% binding to the FcR compared to a native sequence, e.g. as determined by techniques well known in the art. In other embodiments the variant will exhibit enhanced binding as compared to the native immunoglobulin Fc domain. It will be appreciated that these types of Fc variants may advantageously be used to enhance the effective anti-neoplastic properties of the disclosed antibodies. In yet other embodiments, such alterations lead to increased binding affinity, reduced immunogenicity, increased production, altered glycosylation and/or disulfide bonds (e.g., for conjugation sites), modified binding specificity, increased phagocytosis; and/or down regulation of cell surface receptors (e.g. B cell receptor; BCR), etc.

d. Altered Glycosylation

Still other embodiments comprise one or more engineered glycoforms, i.e., a DLL3 site-specific antibody comprising an altered glycosylation pattern or altered carbohydrate composition that is covalently attached to the protein (e.g., in the Fc domain). See, for example, Shields, R. L. et al. (2002) J. Biol. Chem. 277:26733-26740. Engineered glycoforms may be useful for a variety of purposes, including but not limited to enhancing or reducing effector function, increasing the affinity of the modulator for a target or facilitating production of the modulator. In certain embodiments where reduced effector function is desired, the molecule may be engineered to express an aglycosylated form. Substitutions that may result in elimination of one or more variable region framework glycosylation sites to thereby eliminate glycosylation at that site are well known (see e.g. U.S. Pat. Nos. 5,714,350 and 6,350,861). Conversely, enhanced effector functions or improved binding may be imparted to the Fc containing molecule by engineering in one or more additional glycosylation sites.

Other embodiments include an Fc variant that has an altered glycosylation composition, such as a hypofucosylated antibody having reduced amounts of fucosyl residues or an antibody having increased bisecting GlcNAc structures. Such altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTI11)), by expressing a molecule comprising an Fc region in various organisms or cell lines from various organisms or by modifying carbohydrate(s) after the molecule comprising Fc region has been expressed (see, for example, WO 2012/117002).

e. Additional Processing

The site-specific antibodies or conjugates may be differentially modified during or after production, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Various post-translational modifications also encompassed by the invention include, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. Moreover, the modulators may also be modified with a detectable label, such as an enzymatic, fluorescent, radioisotopic or affinity label to allow for detection and isolation of the modulator.

6. Site-Specific Antibody Characteristics

No matter how obtained or which of the aforementioned forms the site-specific conjugate takes, various embodiments of the disclosed antibodies may exhibit certain characteristics. In selected embodiments, antibody-producing cells (e.g., hybridomas or yeast colonies) may be selected, cloned and further screened for favorable properties including, for example, robust growth, high antibody production and, as discussed in more detail below, desirable site-specific antibody characteristics. In other cases characteristics of the antibody may be imparted or influenced by selecting a particular antigen (e.g., a specific DLL3 isoform) or immunoreactive fragment of the target antigen for inoculation of the animal. In still other embodiments the selected antibodies may be engineered as described above to enhance or refine immunochemical characteristics such as affinity or pharmacokinetics.

a. Neutralizing Antibodies

In certain embodiments, the conjugates will comprise “neutralizing” antibodies or derivatives or fragments thereof. That is, the present invention may comprise antibody molecules that bind specific domains, motifs or epitopes and are capable of blocking, reducing or inhibiting the biological activity of DLL3. More generally the term “neutralizing antibody” refers to an antibody that binds to or interacts with a target molecule or ligand and prevents binding or association of the target molecule to a binding partner such as a receptor or substrate, thereby interrupting a biological response that otherwise would result from the interaction of the molecules.

It will be appreciated that competitive binding assays known in the art may be used to assess the binding and specificity of an antibody or immunologically functional fragment or derivative thereof. With regard to the instant invention an antibody or fragment will be held to inhibit or reduce binding of DLL3 to a binding partner or substrate when an excess of antibody reduces the quantity of binding partner bound to DLL3 by at least about 20%, 30%, 40%, 50%, 60%, 70%, 80%, 85%, 90%, 95%, 97%, 99% or more as measured, for example, by Notch receptor activity or in an in vitro competitive binding assay. In the case of antibodies to DLL3 for example, a neutralizing antibody or antagonist will preferably alter Notch receptor activity by at least about 20%, 30%, 40%, 50%, 60%, 70%, 80%, 85%, 90%, 95%, 97%, 99% or more. It will be appreciated that this modified activity may be measured directly using art-recognized techniques or may be measured by the impact the altered activity has downstream (e.g., oncogenesis, cell survival or activation or suppression of Notch responsive genes). Preferably, the ability of an antibody to neutralize DLL3 activity is assessed by inhibition of DLL3 binding to a Notch receptor or by assessing its ability to relieve DLL3 mediated repression of Notch signaling.

b. Internalizing Antibodies

There is evidence that a substantial portion of expressed DLL3 protein remains associated with the tumorigenic cell surface, thereby allowing for localization and internalization of the disclosed site-specific conjugates. In preferred embodiments such modulators will be associated with, or conjugated to, one or more PBDs through engineered free cysteine site(s) that kill the cell upon internalization. In particularly preferred embodiments the site-specific conjugates will comprise an internalizing ADC.

As used herein, a modulator that “internalizes” is one that is taken up (along with any payload) by the cell upon binding to an associated antigen or receptor. As will be appreciated, the internalizing antibody may, in select embodiments, comprise antibody fragments and derivatives thereof, as well as antibody conjugates comprising a DAR of approximately 2. Internalization may occur in vitro or in vivo. For therapeutic applications, internalization will preferably occur in vivo in a subject in need thereof. The number of site-specific antibody conjugates internalized may be sufficient or adequate to kill an antigen-expressing cell, especially an antigen-expressing cancer stem cell. Depending on the potency of the payload or site-specific antibody conjugate as a whole, in some instances, the uptake of a single engineered antibody molecule into the cell is sufficient to kill the target cell to which the antibody binds. For example, certain PBDs are so highly potent that the internalization of a few molecules of the toxin conjugated to the antibody is sufficient to kill the tumor cell. Whether an antibody internalizes upon binding to a mammalian cell can be determined by various art-recognized assays including those described in the Examples below. Methods of detecting whether an antibody internalizes into a cell are also described in U.S. Pat. No. 7,619,068 which is incorporated herein by reference in its entirety.

c. Depleting Antibodies

In other embodiments the site-specific conjugate will comprise depleting antibodies or derivatives or fragments thereof. The term “depleting” antibody refers to an antibody that preferably binds to or associates with an antigen on or near the cell surface and induces, promotes or causes the death or elimination of the cell (e.g., by CDC, ADCC or introduction of a cytotoxic agent). In preferred embodiments, the selected depleting antibodies will be associated or conjugated to a PBD.

Preferably a depleting antibody will be able to remove, incapacitate, eliminate or kill at least 20%, 30%, 40%, 50%, 60%, 70%, 80%, 85%, 90%, 95%, 97%, or 99% of DLL3 expressing cells in a defined cell population. In some embodiments the cell population may comprise enriched, sectioned, purified or isolated tumor perpetuating cells. In other embodiments the cell population may comprise whole tumor samples or heterogeneous tumor extracts that comprise cancer stem cells. Those skilled in the art will appreciate that standard biochemical techniques may be used to monitor and quantify the depletion of tumorigenic cells or tumor perpetuating cells in accordance with the teachings herein.

d. Binning and Epitope Mapping

It will further be appreciated the disclosed anti-DLL3 site-specific antibody conjugates will associate with, or bind to, discrete epitopes or immunogenic determinants presented by the selected target or fragment thereof. In certain embodiments, epitope or immunogenic determinants include chemically active surface groupings of molecules such as amino acids, sugar side chains, phosphoryl groups, or sulfonyl groups, and, in certain embodiments, may have specific three-dimensional structural characteristics, and/or specific charge characteristics. Thus, as used herein the term “epitope” includes any protein determinant capable of specific binding to an immunoglobulin or T-cell receptor or otherwise interacting with a molecule. In certain embodiments, an antibody is said to specifically bind (or immunospecifically bind or react) an antigen when it preferentially recognizes its target antigen in a complex mixture of proteins and/or macromolecules. In preferred embodiments, an antibody is said to specifically bind an antigen when the equilibrium dissociation constant (K_{D}) is less than or equal to 10⁻⁶M or less than or equal to 10⁻⁷M, more preferably when the equilibrium dissociation constant is less than or equal to 10⁻⁸M, and even more preferably when the dissociation constant is less than or equal to 10⁻⁹M

More directly the term “epitope” is used in its common biochemical sense and refers to that portion of the target antigen capable of being recognized and specifically bound by a particular antibody modulator. When the antigen is a polypeptide such as DLL3, epitopes may generally be formed from both contiguous amino acids and noncontiguous amino acids juxtaposed by tertiary folding of a protein (“conformational epitopes”). In such conformational epitopes the points of interaction occur across amino acid residues on the protein that are linearly separated from one another. Epitopes formed from contiguous amino acids (sometimes referred to as “linear” or “continuous” epitopes) are typically retained upon protein denaturing, whereas epitopes formed by tertiary folding are typically lost upon protein denaturing. In any event an antibody epitope typically includes at least 3, and more usually, at least 5 or 8-10 amino acids in a unique spatial conformation.

In this respect it will be appreciated that, in certain embodiments, an epitope may be associated with, or reside in, one or more regions, domains or motifs of the DLL3 protein (e.g., amino acids 1-618 of isoform 1). As discussed in more detail herein the extracellular region of the DLL3 protein comprises a series of generally recognized domains including six EGF-like domains and a DSL domain. For the purposes of the instant disclosure the term “domain” will be used in accordance with its generally accepted meaning and will be held to refer to an identifiable or definable conserved structural entity within a protein that exhibits a distinctive secondary structure content. In many cases, homologous domains with common functions will usually show sequence similarities and be found in a number of disparate proteins (e.g., EGF-like domains are reportedly found in at least 471 different proteins). Similarly, the art-recognized term “motif” will be used in accordance with its common meaning and shall generally refer to a short, conserved region of a protein that is typically ten to twenty contiguous amino acid residues. As discussed throughout, selected embodiments comprise site-specific antibodies that associate with or bind to an epitope within specific regions, domains or motifs of DLL3.

As discussed in more detail in PCT/US14/17810 particularly preferred epitopes of human DLL3 bound by exemplary site-specific antibody conjugates are set forth in Table 3 immediately below.

TABLE 3

| Antibody Clone | Epitope | SEQ ID NO: |
|---|---|---|
| SC16.23 | Q93, P94, G95, A96, P97 | 3 |
| SC16.34 | G203, R205, P206 | 4 |
| SC16.56 | G203, R205, P206 | 4 |

In any event once a desired epitope on an antigen is determined, it is possible to generate antibodies to that epitope, e.g., by immunizing with a peptide comprising the epitope using techniques described in the present invention. Alternatively, during the discovery process, the generation and characterization of antibodies may elucidate information about desirable epitopes located in specific domains or motifs. From this information, it is then possible to competitively screen antibodies for binding to the same epitope. An approach to achieve this is to conduct competition studies to find antibodies that competitively bind with one another, i.e. the antibodies compete for binding to the antigen. A high throughput process for binning antibodies based upon their cross-competition is described in WO 03/48731. Other methods of binning or domain level or epitope mapping comprising antibody competition or antigen fragment expression on yeast are well known in the art.

As used herein, the term “binning” refers to methods used to group or classify antibodies based on their antigen binding characteristics and competition. While the techniques are useful for defining and categorizing modulators of the instant invention, the bins do not always directly correlate with epitopes and such initial determinations of epitope binding may be further refined and confirmed by other art-recognized methodology as described herein. However, as discussed herein, empirical assignment of antibody modulators to individual bins provides information that may be indicative of the therapeutic potential of the disclosed modulators.

More specifically, one can determine whether a selected reference antibody (or fragment thereof) binds to the same epitope or cross competes for binding with a second test antibody (i.e., is in the same bin) by using methods known in the art and set forth in the Examples herein. In one embodiment, a reference antibody modulator is associated with DLL3 antigen under saturating conditions and then the ability of a secondary or test antibody modulator to bind to DLL3 is determined using standard immunochemical techniques. If the test antibody is able to substantially bind to DLL3 at the same time as the reference anti-DLL3 antibody, then the secondary or test antibody binds to a different epitope than the primary or reference antibody. However, if the test antibody is not able to substantially bind to DLL3 at the same time, then the test antibody binds to the same epitope, an overlapping epitope, or an epitope that is in close proximity (at least sterically) to the epitope bound by the primary antibody. That is, the test antibody competes for antigen binding and is in the same bin as the reference antibody.

The term “compete” or “competing antibody” when used in the context of the disclosed antibodies means competition between antibodies as determined by an assay in which a test antibody or immunologically functional fragment under test prevents or inhibits specific binding of a reference antibody to a common antigen. Typically, such an assay involves the use of purified antigen (e.g., DLL3 or a domain or fragment thereof) bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess and/or allowed to bind first. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more.

Conversely, when the reference antibody is bound it will preferably inhibit binding of a subsequently added test antibody (i.e., a DLL3 modulator) by at least 30%, 40%, 45%, 50%, 55%, 60%, 65%, 70% or 75%. In some instance, binding of the test antibody is inhibited by at least 80%, 85%, 90%, 95%, or 97% or more.

With regard to the instant invention, and as set forth in PCT/US14/17810 which is incorporated herein as to the anti-DLL3 antibody bins, it has been determined (via surface plasmon resonance or bio-layer interferometry) that the extracellular domain of DLL3 defines at least nine bins by competitive binding termed “bin A” to “bin I” herein. Given the resolution provided by modulator binning techniques, it is believed that these nine bins comprise the majority of the bins that are present in the extracellular region of the DLL3 protein.

In this respect, and as known in the art the desired binning or competitive binding data can be obtained using solid phase direct or indirect radioimmunoassay (RIA), solid phase direct or indirect enzyme immunoassay (EIA or ELISA), sandwich competition assay, a Biacore™ 2000 system (i.e., surface plasmon resonance—GE Healthcare), a ForteBio® Analyzer (i.e., bio-layer interferometry—ForteBio, Inc.) or flow cytometric methodology. The term “surface plasmon resonance,” as used herein, refers to an optical phenomenon that allows for the analysis of real-time specific interactions by detection of alterations in protein concentrations within a biosensor matrix. The term “bio-layer interferometry” refers to an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. In particularly preferred embodiments the analysis (whether surface plasmon resonance, bio-layer interferometry or flow cytometry) is performed using a Biacore or ForteBio instrument or a flow cytometer (e.g., FACSAria II) as known in the art.

In order to further characterize the epitopes that the disclosed DLL3 antibody modulators associate with or bind to, domain-level epitope mapping may be performed using a modification of the protocol described by Cochran et al. (J Immunol Methods. 287 (1-2):147-158 (2004) which is incorporated herein by reference). Briefly, individual domains of DLL3 comprising specific amino acid sequences were expressed on the surface of yeast and binding by each DLL3 antibody was determined through flow cytometry.

Other compatible epitope mapping techniques include alanine scanning mutants, peptide blots (Reineke (2004) Methods Mol Biol 248:443-63) (herein specifically incorporated by reference in its entirety), or peptide cleavage analysis. In addition, methods such as epitope excision, epitope extraction and chemical modification of antigens can be employed (Tomer (2000) Protein Science 9: 487-496) (herein specifically incorporated by reference in its entirety). In other embodiments Modification-Assisted Profiling (MAP), also known as Antigen Structure-based Antibody Profiling (ASAP) provides a method that categorizes large numbers of monoclonal antibodies (mAbs) directed against the same antigen according to the similarities of the binding profile of each antibody to chemically or enzymatically modified antigen surfaces (U.S.P.N. 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. It will be appreciated that MAP may be used to sort the hDLL3 antibody modulators of the invention into groups of antibodies binding different epitopes

Agents useful for altering the structure of the immobilized antigen include enzymes such as proteolytic enzymes (e.g., trypsin, endoproteinase Glu-C, endoproteinase Asp-N, chymotrypsin, etc.). Agents useful for altering the structure of the immobilized antigen may also be chemical agents, such as, succinimidyl esters and their derivatives, primary amine-containing compounds, hydrazines and carbohydrazines, free amino acids, etc.

The antigen protein may be immobilized on either biosensor chip surfaces or polystyrene beads. The latter can be processed with, for example, an assay such as multiplex LUMINEX™ detection assay (Luminex Corp.). Because of the capacity of LUMINEX to handle multiplex analysis with up to 100 different types of beads, LUMINEX provides almost unlimited antigen surfaces with various modifications, resulting in improved resolution in antibody epitope profiling over a biosensor assay.

e. Binding Affinity

Besides epitope specificity the disclosed site-specific antibodies may be characterized using physical characteristics such as, for example, binding affinities. In this regard the present invention further encompasses the use of antibodies that have a high binding affinity for one or more DLL3 isoforms or, in the case of pan-antibodies, more than one member of the DLL family. As used herein, the term “high affinity” for an IgG antibody refers to an antibody having a K_{D} of 10⁻⁸M or less, more preferably 10⁻⁹M or less and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷M or less, more preferably 10⁻⁸M or less, even more preferably 10⁻⁹M or less.

The term “K_{D}”, as used herein, is intended to refer to the dissociation constant of a particular antibody-antigen interaction. An antibody of the invention is said to immunospecifically bind its target antigen when the dissociation constant K_{D} (k_{off}/kₒₙ) is ≦10⁻⁷M. The antibody specifically binds antigen with high affinity when the K_{D} is ≦5×10⁻⁹M, and with very high affinity when the K_{D} is ≦5×10⁻¹⁰M. In one embodiment of the invention, the antibody has a K_{D} of ≦10⁻⁹M and an off-rate of about 1×10⁻⁴/sec. In one embodiment of the invention, the off-rate is <1×10⁻⁵/sec. In other embodiments of the invention, the antibodies will bind to DLL3 with a K_{D} of between about 10⁻⁷M and 10⁻¹⁰M, and in yet another embodiment it will bind with a K_{D}≦2×10⁻¹⁰M. Still other selected embodiments of the present invention comprise antibodies that have a disassociation constant or K_{D} (k_{off}/kₒₙ) of less than 10⁻²M, less than 5×10⁻²M, less than 10⁻³M, less than 5×10⁻³M, less than 10⁻⁴M, less than 5×10⁻⁴M, less than 10⁻⁵M, less than 5×10⁻⁵M, less than 10⁻⁶M, less than 5×10⁻⁶M, less than 10⁻⁷M, less than 5×10⁻⁷M, less than 10⁻⁸M, less than 5×10⁻⁸M, less than 10⁻⁹M, less than 5×10⁻⁹M, less than 10⁻¹⁰M, less than 5×10¹⁰M, less than 10⁻¹¹M, less than 5×10⁻¹¹M, less than 10⁻¹²M, less than 5×10⁻¹²M, less than 10⁻¹³M, less than 5×10⁻¹³M, less than 10⁻¹⁴M, less than 5×10⁻¹⁴M, less than 10⁻¹⁵M or less than 5×10⁻¹⁵M.

In specific embodiments, an antibody of the invention that immunospecifically binds to DLL3 has an association rate constant or kₒₙ (or kₐ) rate (DLL3 (Ab)+antigen (Ag)^{k}_{off}←Ab-Ag) of at least 10⁵M⁻¹s⁻¹, at least 2×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹.

In another embodiment, an antibody of the invention that immunospecifically binds to DLL3 has a disassociation rate constant or k_{off} (or k_{d}) rate (DLL3 (Ab)+antigen (Ag)^{k}_{off}←Ab-Ag) of less than 10⁻¹s⁻¹, less than 5×10⁻¹s⁻¹, less than 10⁻²s⁻¹, less than 5×10⁻²s⁻¹, less than 10⁻³s⁻¹, less than 5×10⁻³s⁻¹, less than 10⁻⁴s⁻¹, less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹ less than 10⁻⁷S⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹ or less than 10⁻¹⁰s⁻¹.

In other selected embodiments of the present invention anti-DLL3 antibodies will have an affinity constant or Kₐ (kₒₙ/k_{off}) of at least 10²M⁻¹, at least 5×10²M⁻¹, at least 10³M⁻¹, at least 5×10³M⁻¹, at least 10⁴M⁻¹, at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹⁰M⁻¹, at least 5×10¹⁰M⁻¹, at least 10¹¹M⁻¹, at least 5×10¹¹M⁻¹, at least 10¹²M⁻¹, at least 5×10¹²M⁻¹, at least 10¹³M⁻¹, at least 5×10¹³M⁻¹, at least 10¹⁴M⁻¹, at least 5×10¹⁴M⁻¹, at least 10¹⁵M⁻¹ or at least 5×10¹⁵M⁻¹.

Besides the aforementioned modulator characteristics antibodies of the instant invention may further be characterized using additional physical characteristics including, for example, thermal stability (i.e, melting temperature; Tm), and isoelectric points. (See, e.g., Bjellqvist et al., 1993, Electrophoresis 14:1023; Vermeer et al., 2000, Biophys. J. 78:394-404; Vermeer et al., 2000, Biophys. J. 79: 2150-2154 each of which is incorporated herein by reference).

### IV. Site-Specific Antibody Drug Conjugates

It will be appreciated that the site-specific anti-DLL3 conjugates of the instant invention comprise a site-specific anti-DLL3 antibody covalently linked (preferably through a linker moiety) to one or more PBD drug payload(s) via unpaired cysteines. As discussed herein the site-specific anti-DLL3 conjugates of the instant invention may be used to provide cytotoxic PBDs at the target location (e.g., tumorigenic cells). This is advantageously achieved by the disclosed site-specific ADCs which direct the bound drug payload to the target site in a relatively unreactive, non-toxic state before releasing and activating the drug payload. As discussed herein this targeted release of the toxic payload is largely achieved through the stable site-specific conjugation of the payloads via one or more free cysteines and the relatively homogeneous composition of the ADC preparations which minimize over-conjugated toxic species. Coupled with drug linkers that are designed to largely release the PBD payload once it has been delivered to the tumor site, the conjugates of the instant invention can substantially reduce undesirable non-specific toxicity. This advantageously provides for relatively high levels of the active PBD cytotoxin at the tumor site while minimizing exposure of non-targeted cells and tissue thereby providing an enhanced therapeutic index when compared with conventional drug conjugates.

More specifically, once the disclosed site-specific antibodies of the invention have been generated and/or fabricated and selected according to the teachings herein they may be linked with, fused to, conjugated to, or otherwise associated with one or more PBDs as described below. As used herein the term “conjugate” or “site-specific conjugate” or “antibody conjugate” will be used broadly and held to mean any PBD associated with the disclosed site-specific antibodies via an unpaired cysteine regardless of the method of association. Moreover, as indicated above the selected conjugate may be associated with, or linked to, the engineered antibody and exhibit various stoichiometric molar ratios depending, at least in part, on the method used to effect the conjugation and the number of free cysteines.

In this regard it will be appreciated that, unless otherwise dictated by context, the site-specific anti-DLL3 conjugates of the instant invention may be represented by the formula:

Ab-[L-D]n or a pharmaceutically acceptable salt thereof wherein
a) Ab comprises a DLL3 antibody comprising one or more unpaired cysteines;
b) L comprises an optional linker;
c) D comprises a PBD; and
d) n is an integer from about 1 to about 8.

Those of skill in the art will appreciate that site-specific conjugates according to the aforementioned formula may be fabricated using a number of different linkers and PBDs and that fabrication or conjunction methodology will vary depending on the selection of components. As such, any PBD or PBD-linker compound that reacts with a thiol on the reactive cysteine(s) of the site-specific antibody is compatible with the teachings herein. Similarly, any reaction conditions that allow for site-specific conjugation of the selected PBD to the DLL3 antibody are within the scope of the present invention. Notwithstanding the foregoing, particularly preferred embodiments of the instant invention comprise selective conjugation of the PBD or PBD-linker using stabilization agents in combination with mild reducing agents as described herein and set forth in the Examples below. Such reaction conditions tend to provide more homogeneous preparations with less non-specific conjugation and contaminants and correspondingly less toxicity.

Particularly preferred site-specific ADCs according to the above formula comprise the following:

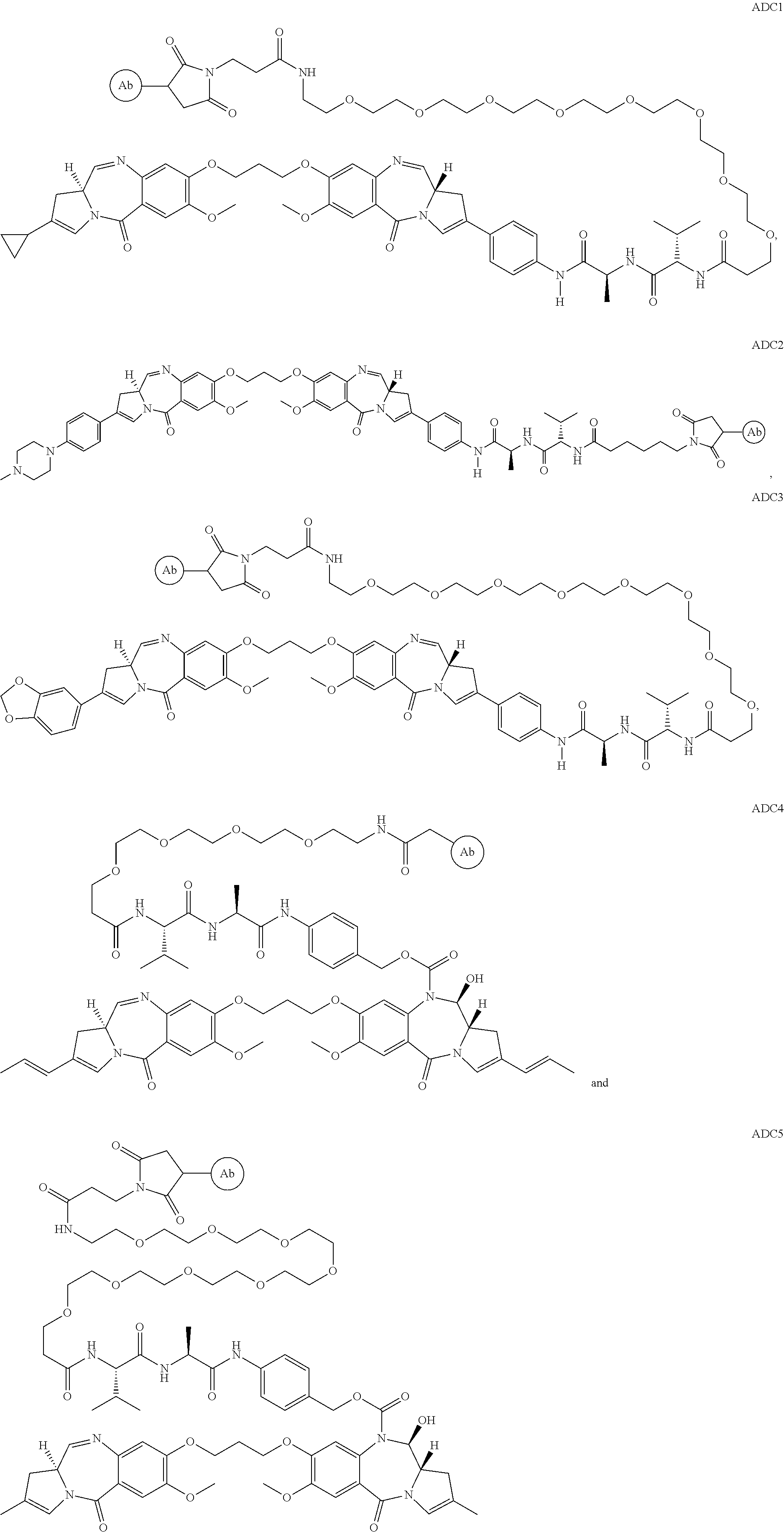

wherein Ab comprises a DLL3 antibody comprising one or more free cysteines and n is an integer between 1 and 20.

As used herein the terms “site-specific conjugate” or “antibody conjugate” or “DLL3 conjugate” or “site-specific ADC” may be used interchangeably unless otherwise dictated by context and held to comprise any of ADC 1, ADC 2, ADC 3, ADC 4 or ADC 5. Along with art recognized techniques, it will be appreciated that novel reaction conditions disclosed herein can be used to conjugate the selected site-specific antibody and the PBD-linker to provide the desired site-specific ADC. In this regard preferred selective reduction techniques are set forth in Examples 6-8 below. Moreover, by using the selective reduction techniques set forth herein in combination with particular site-specific antibody constructs, highly homogeneous ADC preparations exhibiting tightly defined stoichiometric DAR values and payload positioning along with relatively low non-specific conjugation may be provided.

1. Pyrrolobenzodiazepines

As indicated throughout the instant specification embodiments of the instant invention are directed to site-specific conjugated anti-DLL3 antibodies that comprise pyrrolobenzodiazepine (PBD) as a cytotoxic agent. It will be appreciated that PBDs are alkylating agents that exert antitumor activity by covalently binding to DNA in the minor groove and inhibiting nucleic acid synthesis. In this respect PBDs have been shown to have potent antitumor properties while exhibiting minimal bone marrow depression. PBDs compatible with the present invention may be linked to the DLL3 modulator using any one of several types of linker (e.g., a peptidyl linker comprising a maleimido moiety with a free sulfhydryl) and, in certain embodiments are dimeric in form (Le PBD dimers). PBDs are of the general structure:

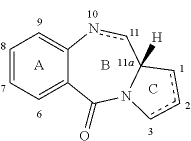

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as cytotoxic agents. As alluded to above, in order to increase their potency PBDs are often used in a dimeric form which may be conjugated to site-specific anti-DLL3 antibodies as described herein. Compatible PBDs (and optional linkers) that may be conjugated to the disclosed site-specific antibodies are described, for example, in U.S. Pat. Nos. 6,362,331, 7,049,311, 7,189,710, 7,429,658, 7,407,951, 7,741,319, 7,557,099, 8,034,808, 8,163,736 U.S.P.N. 2011/0256157 and PCT filings WO2011/130613, WO2011/128650, WO2011/130616 and WO2014/057074 each of which is incorporated herein by reference as to the PBDs disclosed.

In particularly preferred embodiments compatible PBDs that may be conjugated to the disclosed modulators are described, in U.S.P.N. 2011/0256157. In this disclosure, PBD dimers, i.e. those comprising two PBD moieties may be preferred. Thus, preferred conjugates of the present invention are those having the formula (AB) or (AC):

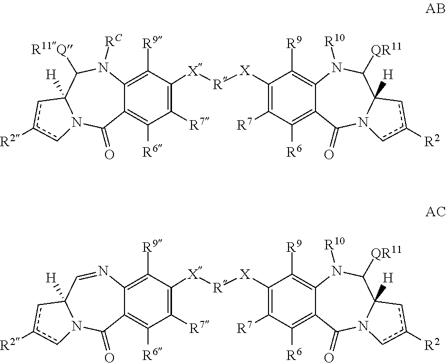

wherein:

the dotted lines indicate the optional presence of a double bond between C1 and C2 or C2 and C3;
R² is independently selected from H, OH, ═O, ═CH₂, CN, R, OR, ═CH—R^{D}, ═C(R^{D})₂, O—SO₂—R, CO₂R and COR, and optionally further selected from halo or dihalo;
where R^{D} is independently selected from R, CO₂R, COR, CHO, CO₂H, and halo;
R⁶ and R⁹ are independently selected from H, R, OH, OR, SH, SR, NH₂, NHR, NRR′, NO₂, Me₃Sn and halo;
R⁷ is independently selected from H, R, OH, OR, SH, SR, NH₂, NHR, NRR′, NO₂, Me₃Sn and halo;
R¹⁰ is a linker connected to a modulator or fragment or derivative thereof, as described above;
Q is independently selected from O, S and NH;
R¹¹ is either H, or R or, where Q is O, SO₃M, where M is a metal cation;
R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring; and

wherein R^{2″}, R^{6″}, R^{7″}, R^{9″}, X″, Q″ and R^{11″} and are as defined according to R², R⁶, R⁷, R⁹, X, Q and R¹¹ respectively, and R^{C} is a capping group.

Double Bond

In one embodiment, there is no double bond present between C1 and C2, and C2 and C3.

In one embodiment, the dotted lines indicate the optional presence of a double bond between C2 and C3, as shown below:

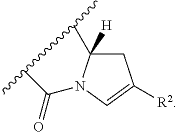

In one embodiment, a double bond is present between C2 and C3 when R² is C₅₋₂₀ aryl or C₁₋₁₂ alkyl.

In one embodiment, the dotted lines indicate the optional presence of a double bond between C1 and C2, as shown below:

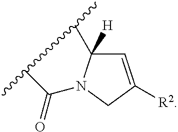

In one embodiment, a double bond is present between C1 and C2 when R² is C₅₋₂₀ aryl or C₁₋₁₂ alkyl.

R²

In one embodiment, R² is independently selected from H, OH, ═O, ═CH₂, CN, R, OR, ═CH—R^{D}, ═C(R^{D})₂, O—SO₂—R, CO₂R and COR, and optionally further selected from halo or dihalo.

In one embodiment, R² is independently selected from H, OH, ═O, ═CH₂, CN, R, OR, ═CH—R^{D}, ═C(R^{D})₂, O—SO₂—R, CO₂R and COR.

In one embodiment, R² is independently selected from H, ═O, ═CH₂, R, ═CH—R^{D}, and ═C(RD)₂.

In one embodiment, R² is independently H.

In one embodiment, R² is independently ═O.

In one embodiment, R² is independently ═CH₂.

In one embodiment, R² is independently ═CH—R^{D}. Within the PBD compound, the group ═CH—R^{D} may have either configuration shown below:

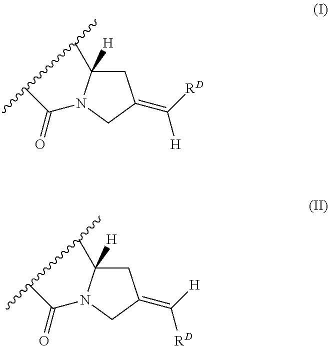

In one embodiment, the configuration is configuration (I).

In one embodiment, R² is independently ═C(R^{D})_{2.}

In one embodiment, R² is independently ═CF₂.

In one embodiment, R² is independently R.

In one embodiment, R² is independently optionally substituted C₅₋₂₀ aryl.

In one embodiment, R² is independently optionally substituted C₁₋₁₂ alkyl.

In one embodiment, R² is independently optionally substituted C₅₋₂₀ aryl.

In one embodiment, R² is independently optionally substituted C₅₋₇ aryl.

In one embodiment, R² is independently optionally substituted C₈₋₁₀ aryl.

In one embodiment, R² is independently optionally substituted phenyl.

In one embodiment, R² is independently optionally substituted napthyl.

In one embodiment, R² is independently optionally substituted pyridyl.

In one embodiment, R² is independently optionally substituted quinolinyl or isoquinolinyl.

In one embodiment, R² bears one to three substituent groups, with 1 and 2 being more preferred, and singly substituted groups being most preferred. The substituents may be any position.

Where R² is a C₅₋₇ aryl group, a single substituent is preferably on a ring atom that is not adjacent the bond to the remainder of the compound, i.e. it is preferably β or γ to the bond to the remainder of the compound. Therefore, where the C₅₋₇ aryl group is phenyl, the substituent is preferably in the meta- or para-positions, and more preferably is in the para-position.

In one embodiment, R² is selected from:

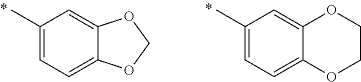

where the asterisk indicates the point of attachment.

Where R² is a C₈₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

In one embodiment, where R² is optionally substituted, the substituents are selected from those substituents given in the substituent section below.

Where R is optionally substituted, the substituents are preferably selected from:
Halo, Hydroxyl, Ether, Formyl, Acyl, Carboxy, Ester, Acyloxy, Amino, Amido, Acylamido, Aminocarbonyloxy, Ureido, Nitro, Cyano and Thioether.

In one embodiment, where R or R² is optionally substituted, the substituents are selected from the group consisting of R, OR, SR, NRR′, NO₂, halo, CO₂R, COR, CONH₂, CONHR, and CONRR′.

Where R² is C₁₋₁₂ alkyl, the optional substituent may additionally include C₃₋₂₀ heterocyclyl and C₅₋₂₀ aryl groups.

Where R² is C₃₋₂₀ heterocyclyl, the optional substituent may additionally include C₁₋₁₂ alkyl and C₅₋₂₀ aryl groups.

Where R² is C₅₋₂₀ aryl groups, the optional substituent may additionally include C₃₋₂₀ heterocyclyl and C₁₋₁₂alkyl groups.

It is understood that the term “alkyl” encompasses the sub-classes alkenyl and alkynyl as well as cycloalkyl. Thus, where R² is optionally substituted C₁₋₁₂ alkyl, it is understood that the alkyl group optionally contains one or more carbon-carbon double or triple bonds, which may form part of a conjugated system. In one embodiment, the optionally substituted C₁₋₁₂ alkyl group contains at least one carbon-carbon double or triple bond, and this bond is conjugated with a double bond present between C1 and C2, or C2 and C3. In one embodiment, the C₁₋₁₂ alkyl group is a group selected from saturated C₁₋₁₂ alkyl, C₂₋₁₂ alkenyl, C₂₋₁₂ alkynyl and C₃₋₁₂ cycloalkyl.

If a substituent on R² is halo, it is preferably F or Cl, more preferably Cl.

If a substituent on R² is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy).

If a substituent on R² is C₁₋₇ alkyl, it may preferably be a C₁₋₄ alkyl group (e.g. methyl, ethyl, propyl, butyl).

If a substituent on R² is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups.

If a substituent on R² is bis-oxy-C₁₋₃ alkylene, this is preferably bis-oxy-methylene or bis-oxy-ethylene.

Particularly preferred substituents for R² include methoxy, ethoxy, fluoro, chloro, cyano, bis-oxy-methylene, methyl-piperazinyl, morpholino and methyl-thienyl.

Particularly preferred substituted R² groups include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthienyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl.

In one embodiment, R² is halo or dihalo. In one embodiment, R² is —F or —F₂, which substituents are illustrated below as (III) and (IV) respectively:

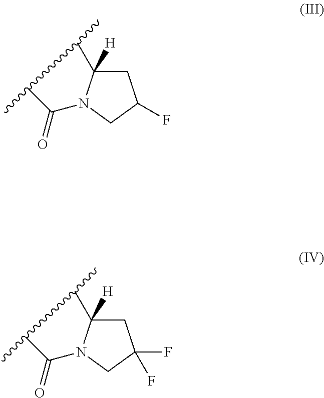

R^{D}

In one embodiment, R^{D} is independently selected from R, CO₂R, COR, CHO, CO₂H, and halo.

In one embodiment, R^{D} is independently R.

In one embodiment, R^{D} is independently halo.

R⁶

In one embodiment, R⁶ is independently selected from H, R, OH, OR, SH, SR, NH₂, NHR, NRR′, NO₂, Me₃Sn— and Halo.

In one embodiment, R⁶ is independently selected from H, OH, OR, SH, NH₂, NO₂ and Halo.

In one embodiment, R⁶ is independently selected from H and Halo.

In one embodiment, R⁶ is independently H.

In one embodiment, R⁶ and R⁷ together form a group —O—(CH₂)ₚ—O—, where p is 1 or 2.

R⁷

R⁷ is independently selected from H, R, OH, OR, SH, SR, NH₂, NHR, NRR′, NO₂, Me₃Sn and halo.

In one embodiment, R⁷ is independently OR.

In one embodiment, R⁷ is independently OR^{7A}, where R^{7A} is independently optionally substituted C₁₋₆ alkyl.

In one embodiment, R^{7A} is independently optionally substituted saturated C₁₋₆ alkyl.

In one embodiment, R^{7A} is independently optionally substituted C₂₋₄ alkenyl.

In one embodiment, R^{7A} is independently Me.

In one embodiment, R^{7A} is independently CH₂Ph.

In one embodiment, R^{7A} is independently allyl.

In one embodiment, the compound is a dimer where the R⁷ groups of each monomer form together a dimer bridge having the formula X—R″—X linking the monomers.

R⁸

In one embodiment, the compound is a dimer where the R⁸ groups of each monomer form together a dimer bridge having the formula X—R″—X linking the monomers.

In one embodiment, R⁸ is independently OR^{8A}, where R^{8A} is independently optionally substituted C₁₋₄ alkyl.

In one embodiment, R^{8A} is independently optionally substituted saturated C₁₋₆ alkyl or optionally substituted C₂₋₄ alkenyl.

In one embodiment, R^{8A} is independently Me.

In one embodiment, R^{8A} is independently CH₂Ph.

In one embodiment, R^{8A} is independently allyl.

In one embodiment, R and R⁷ together form a group —O—(CH₂)ₚ—O—, where p is 1 or 2.

In one embodiment, R and R⁹ together form a group —O—(CH₂)ₚ—O—, where p is 1 or 2.

R⁹

In one embodiment, R⁹ is independently selected from H, R, OH, OR, SH, SR, NH₂, NHR, NRR′, NO₂, Me₃Sn— and Halo.

In one embodiment, R⁹ is independently H.

In one embodiment, R⁹ is independently R or OR.

R and R′

In one embodiment, R is independently selected from optionally substituted C₁₋₁₂ alkyl, C₃₋₂₀ heterocyclyl and C₅₋₂₀ aryl groups. These groups are each defined in the substituents section below.

In one embodiment, R is independently optionally substituted C₁₋₁₂ alkyl.

In one embodiment, R is independently optionally substituted C₃₋₂₀ heterocyclyl.

In one embodiment, R is independently optionally substituted C₅₋₂₀ aryl.

In one embodiment, R is independently optionally substituted C₁₋₁₂ alkyl.

Described above in relation to R² are various embodiments relating to preferred alkyl and aryl groups and the identity and number of optional substituents. The preferences set out for R² as it applies to R are applicable, where appropriate, to all other groups R, for examples where R⁶, R⁷, R⁸ or R⁹ is R.

The preferences for R apply also to R′.

In some embodiments of the invention there is provided a compound having a substituent group —NRR′. In one embodiment, R and R′ together with the nitrogen atom to which they are attached form an optionally substituted 4-, 5-, 6- or 7-membered heterocyclic ring. The ring may contain a further heteroatom, for example N, O or S.

In one embodiment, the heterocyclic ring is itself substituted with a group R. Where a further N heteroatom is present, the substituent may be on the N heteroatom.

R″

R″ is a C₃₋₁₂ alkylene group, which chain may be interrupted by one or more heteroatoms, e.g. O, S, N(H), NMe and/or aromatic rings, e.g. benzene or pyridine, which rings are optionally substituted.

In one embodiment, R″ is a C₃₋₁₂ alkylene group, which chain may be interrupted by one or more heteroatoms and/or aromatic rings, e.g. benzene or pyridine.

In one embodiment, the alkylene group is optionally interrupted by one or more heteroatoms selected from O, S, and NMe and/or aromatic rings, which rings are optionally substituted.

In one embodiment, the aromatic ring is a C₅₋₂₀ arylene group, where arylene pertains to a divalent moiety obtained by removing two hydrogen atoms from two aromatic ring atoms of an aromatic compound, which moiety has from 5 to 20 ring atoms.

In one embodiment, R″ is a C₃₋₁₂ alkylene group, which chain may be interrupted by one or more heteroatoms, e.g. O, S, N(H), NMe and/or aromatic rings, e.g. benzene or pyridine, which rings are optionally substituted by NH₂.

In one embodiment, R″ is a C₃₋₁₂ alkylene group.

In one embodiment, R″ is selected from a C₃, C₅, C₇, C₉ and a C₁₁ alkylene group.

In one embodiment, R″ is selected from a C₃, C₅ and a C₇ alkylene group.

In one embodiment, R″ is selected from a C₃ and a C₅ alkylene group.

In one embodiment, R″ is a C₃ alkylene group.

In one embodiment, R″ is a C₅ alkylene group.

The alkylene groups listed above may be optionally interrupted by one or more heteroatoms and/or aromatic rings, e.g. benzene or pyridine, which rings are optionally substituted.

The alkylene groups listed above may be optionally interrupted by one or more heteroatoms and/or aromatic rings, e.g. benzene or pyridine.

The alkylene groups listed above may be unsubstituted linear aliphatic alkylene groups.

X

In one embodiment, X is selected from O, S, or N(H).

Preferably, X is O.

R¹⁰

Preferably compatible linkers such as those described below attach to the DLL3 site-specific antibody to the PBD drug moiety through covalent bond(s) at the R¹⁰ position (i.e., N10).

In addition to the aforementioned PBDs a number of PBDs have been shown to be particularly active and may be used in conjunction with the instant invention. To this end particularly preferred embodiments the site-specific conjugates (i.e., ADC 1-5) may comprise a PBD compound as set forth immediately below as PBD 1-5. The synthesis of each of PBD 1-5 as a component of drug-linker compounds is presented in great detail in PCT/US14/17810 which is hereby incorporated by reference as to such synthesis. In view of PCT/US14/17810 the toxic compounds that comprise preferred payloads of the site-specific ADCs of the present invention could readily be generated and employed as set forth herein. The PBD compounds that are released from ADCs 1-5 upon cleavage of the linker are set forth immediately below:

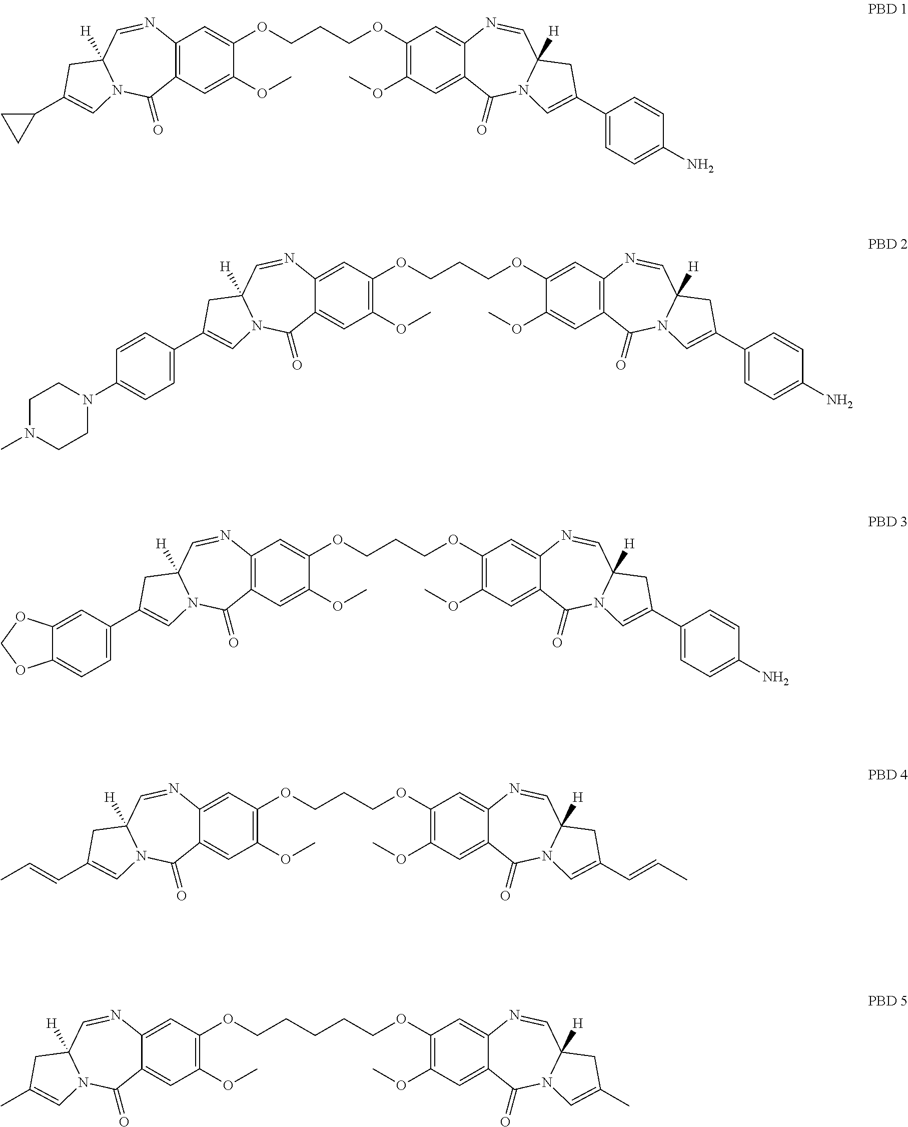

Delivery and release of such compounds at the tumor site(s) may prove clinically effective in treating or managing proliferative disorders in accordance with the instant disclosure. With regard to the compounds it will be appreciated that each of the disclosed PBDs have two sp² centers in each C-ring, which may allow for stronger binding in the minor groove of DNA (and hence greater toxicity), than for compounds with only one sp² centre in each C-ring. Thus, when used in DLL3 ADCs as set forth herein the disclosed PBDs may prove to be particularly effective for the treatment of proliferative disorders.

The foregoing provides exemplary PBD compounds that are compatible with the instant invention and is in no way meant to be limiting as to other PBDs that may be successfully incorporated in site-specific anti-DLL3 conjugates according to the teachings herein. Rather, any PBD that may be conjugated to a site-specific antibody as described herein and set forth in the Examples below is compatible with the disclosed conjugates expressly with the metes and bounds of the invention.

2. Linker Compounds

As with PBDs numerous linker compounds are compatible with the instant invention and may be successfully used in combination with the teachings herein to provide the disclosed anti-DLL3 site-specific conjugates. In a broad sense the linkers merely need to covalently bind with the reactive thiol provided by the free cysteine and the selected PBD compound. As briefly alluded to above in selected embodiments the selected linker will covalently bind to the N10 position of the dimeric PBD. However, in other embodiments compatible linkers may covalently bind the selected PBD at any accessible site on one of the rings or a substituent appended to the rings. Accordingly, any linker that reacts with the free cysteine(s) of the engineered antibody and may be used to provide the relatively stable site-specific conjugates of the instant invention is compatible with the teachings herein.

With regard to effectively binding to the selectively reduced free cysteine a number of art-recognized compounds take advantage of the good nucleophilicity of thiols and thus are available for use as part of a compatible linker. Free cysteine conjugation reactions include, but are not limited to, thiol-maleimide, thiol-halogeno (acyl halide), thiol-ene, thiol-yne, thiol-vinylsulfone, thiol-bisulfone, thiol-thiosulfonate, thiol-pyridyl disulfide and thiol-parafluoro reactions. As further discussed herein and shown in the Examples below, thiol-maleimide bioconjugation is one of the most widely used approaches due to its fast reaction rates and mild conjugation conditions. One issue with this approach is possibility of the retro-Michael reaction and loss or transfer of the maleimido-linked payload from the antibody or other target protein to other proteins in the plasma, such as, for example, human serum albumin. However, as specifically shown in Example 12 the use of selective reduction and site-specific antibodies as set forth herein may be used to stabilize the conjugate and reduce this undesired transfer. Thiol-acyl halide reactions provide bioconjugates that cannot undergo retro-Michael reaction and therefore are more stable. However, the thiol-halide reactions in general have slower reaction rates compared to maleimide-based conjugations and are thus not as efficient. Thiol-pyridyl disulfide reaction is another popular bioconjugation route. The pyridyl disulfide undergoes fast exchange with free thiol resulting in the mixed disulfide and release of pyridine-2-thione. Mixed disulfides can be cleaved in the reductive cell environment releasing the payload. Other approaches gaining more attention in bioconjugation are thiol-vinylsulfone and thiol-bisulfone reactions, each of which are compatible with the teachings herein and expressly included within the scope of the invention.

With regard to compatible linkers the compounds incorporated into the disclosed ADCs are preferably stable extracellularly, prevent aggregation of ADC molecules and keep the ADC freely soluble in aqueous media and in a monomeric state. Before transport or delivery into a cell, the antibody-drug conjugate is preferably stable and remains intact, i.e. the antibody remains linked to the drug moiety. While the linkers are stable outside the target cell they are designed to be cleaved or degraded at some efficacious rate inside the cell. Accordingly an effective linker will: (i) maintain the specific binding properties of the antibody; (ii) allow intracellular delivery of the conjugate or drug moiety; (iii) remain stable and intact, i.e. not cleaved or degraded, until the conjugate has been delivered or transported to its targeted site; and (iv) maintain a cytotoxic, cell-killing effect or a cytostatic effect of the drug moiety. As discussed in more detail in the appended Examples stability of the ADC may be measured by standard analytical techniques such as mass spectroscopy, hydrophobic interaction chromatography (HIC), HPLC, and the separation/analysis technique LC/MS. As set forth above covalent attachment of the antibody and the drug moiety requires the linker to have two reactive functional groups, i.e. bivalency in a reactive sense. Bivalent linker reagents which are useful to attach two or more functional or biologically active moieties, such as PBDs and site-specific antibodies are known, and methods have been described to provide their resulting conjugates.

Linkers compatible with the present invention may broadly be classified as cleavable and non-cleavable linkers. Cleavable linkers, which may include acid-labile linkers, protease cleavable linkers and disulfide linkers, take advantage of internalization by the target cell and cleavage in the endosomal-lysosomal pathway. Release and activation of the cytotoxin relies on endosome/lysosome acidic compartments that facilitate cleavage of acid-labile chemical linkages such as hydrazone or oxime. If a lysosomal-specific protease cleavage site is engineered into the linker the cytotoxins will be released in proximity to their intracellular targets. Alternatively, linkers containing mixed disulfides provide an approach by which cytotoxic payloads are released intracellularly as they are selectively cleaved in the reducing environment of the cell, but not in the oxygen-rich environment in the bloodstream. By way of contrast, compatible non-cleavable linkers containing amide linked polyethyleneglycol or alkyl spacers liberate toxic payloads during lysosomal degradation of the antibody-drug conjugate within the target cell. In some respects the selection of linker will depend on the particular PBD used in the site-specific conjugate.

Accordingly, certain embodiments of the invention comprise a linker that is cleavable by a cleaving agent that is present in the intracellular environment (e.g., within a lysosome or endosome or caveolae). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, each of which is known to hydrolyze dipeptide drug derivatives resulting in the release of active drug inside target cells.

Exemplary peptidyl linkers that are cleavable by the thiol-dependent protease Cathepsin-B are peptides comprising Phe-Leu since cathepsin-B has been found to be highly expressed in cancerous tissue. Other examples of such linkers are described, for example, in U.S. Pat. No. 6,214,345 and U.S.P.N. 2012/0078028 each of which incorporated herein by reference in its entirety. In a specific preferred embodiment, the peptidyl linker cleavable by an intracellular protease is a Val-Cit linker, a Val-Ala linker or a Phe-Lys linker such as is described in U.S. Pat. No. 6,214,345. One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, oxime, semicarbazone, thiosemicarbazone, cis-aconitic amide, orthoester, acetal, ketal, or the like) can be used (See, e.g., U.S. Pat. Nos. 5,122,368; 5,824,805; 5,622,929). Such linkers are relatively stable under neutral pH conditions, such as those in the blood, but are unstable at below pH 5.5 or 5.0, the approximate pH of the lysosomne.

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio)toluene). In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In particularly preferred embodiments (set forth in U.S.P.N. 2011/0256157 which is incorporated herein as to the linkers) compatible peptidyl linkers will comprise:

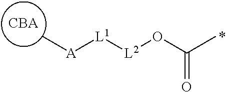

where the asterisk indicates the point of attachment to the cytotoxic PBD, CBA is the site-specific antibody, L¹ is a linker, A is a connecting group connecting L¹ to an unpaired cysteine on the site specific antibody, L² is a covalent bond or together with —OC(═O)— forms a self-immolative linker, and L¹ or L² is a cleavable linker.

L¹ is preferably the cleavable linker, and may be referred to as a trigger for activation of the linker for cleavage.

The nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidising conditions may also find use in the present invention.

L¹ may comprise a contiguous sequence of amino acids. The amino acid sequence may be the target substrate for enzymatic cleavage, thereby allowing release of R¹⁰ from the N10 position.

In one embodiment, L¹ is cleavable by the action of an enzyme. In one embodiment, the enzyme is an esterase or a peptidase.

In one embodiment, L¹ comprises a dipeptide. The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a cathepsin labile linker, the dipeptide may be the site of action for cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂—CO—, is selected from:
-Phe-Lys-, -Val-Ala-, -Val-Lys-, -Ala-Lys-, -Val-Cit-, -Phe-Cit-, -Leu-Cit-, -Ile-Cit-, -Phe-Arg- and -Trp-Cit- where Cit is citrulline.

Preferably, the group —X₁—X₂— in dipeptide, —NH—X₁—X₂—CO—, is selected from:
-Phe-Lys-, -Val-Ala-, -Val-Lys-, -Ala-Lys-, and -Val-Cit-.

Most preferably, the group —X₁—X₂— in dipeptide, —NH—X₁—X₂—CO—, is -Phe-Lys- or -Val-Ala-.

In one embodiment, L² is present and together with —C(═O)O— forms a self-immolative linker. In one embodiment, L² is a substrate for enzymatic activity, thereby allowing release of R¹⁰ from the N10 position.

In one embodiment, where L¹ is cleavable by the action of an enzyme and L² is present, the enzyme cleaves the bond between L¹ and L².

L¹ and L², where present, may be connected by a bond selected from:
—C(═O)NH—, —C(═O)O—, —NHC(═O)—, —OC(═O)—, —OC(═O)O—, —NHC(═O)O—, —OC(═O)NH—, and —NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

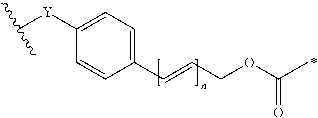

where the asterisk indicates the point of attachment to the drug or cytotoxic agent position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents as described herein. In one embodiment, the phenylene group is optionally substituted with halo, NO₂, R or OR.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

In another particularly preferred embodiments the linker may include a self-immolative linker and the dipeptide together form the group —NH-Val-Ala-CO—NH-PABC-, which is illustrated below:

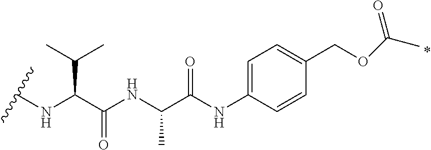 [SMILES: *N[C@H](C(=O)N[C@@H](C)C(=O)Nc1ccc(COC(C)=O)cc1)C(C)C]

where the asterisk indicates the point of attachment to the selected PBD cytotoxic moiety, and the wavy line indicates the point of attachment to the remaining portion of the linker (e.g., the spacer-antibody binding segments) which may be conjugated to the antibody. Upon enzymatic cleavage of the dipeptide the self-immolative linker will allow for clean release of the protected compound (i.e., the toxic PBD) when a remote site is activated, proceeding along the lines shown below:

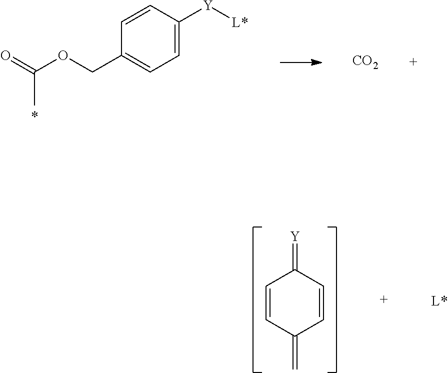

where L* is the activated form of the remaining portion of the linker comprising the now cleaved peptidyl unit. The clean release of PBD 4 and PBD 5 ensure they will maintain the desired toxic activity.

In one embodiment, A is a covalent bond. Thus, L¹ and the cell binding agent are directly connected. For example, where L¹ comprises a contiguous amino acid sequence, the N-terminus of the sequence may connect directly to the free cysteine.

In another embodiment, A is a spacer group. Thus, L¹ and the cell binding agent are indirectly connected.

L¹ and A may be connected by a bond selected from:
—C(═O)NH—, —C(═O)O—, —NHC(═O)—, —OC(═O)—, —OC(═O)O—, —NHC(═O)O—, —OC(═O)NH—, and —NHC(═O)NH—.

As will be discussed in more detail below and set forth in Examples 5-8 below the drug linkers of the instant invention will be linked to reactive thiol nucleophiles on free cysteines. Antibodies may be made reactive for conjugation with linker reagents by treatment with a reducing agent such as DTT or TCEP.

Preferably, the linker contains an electrophilic functional group for reaction with a nucleophilic functional group on the modulator. Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones, carboxyl, and, some of which are exemplified as follows:

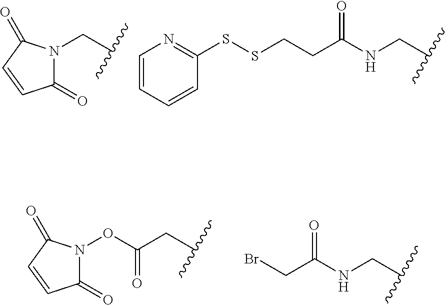

In particularly preferred embodiments the connection between the site-specific antibody and the drug-linker moiety is through a thiol residue of a free cysteine of the engineered DLL3 antibody and a terminal maleimide group of present on the linker. In such embodiments, the connection between the cell binding agent and the drug-linker is:

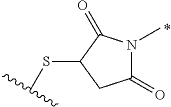 [SMILES: *SC1CC(=O)N(C)C1=O]

where the asterisk indicates the point of attachment to the remaining portion of drug-linker and the wavy line indicates the point of attachment to the remaining portion of the engineered antibody. In this embodiment, the S atom is typically derived from the DLL3 antibody. With regard to ADC 4 above the binding moiety comprises a terminal iodoacetamide that may be reacted with activated thiols to provide the desired site-specific conjugate. The preferred conjugation procedure for this linker is slightly different from the preferred conjugation procedure for the maleimide binding group comprising selective reduction found in the other embodiments and set forth in the Examples below. In any event one skilled in the art could readily conjugate each of the disclosed drug-linker compounds with a compatible anti-DLL3 site-specific antibody in view of the instant disclosure.

3. Conjugation

As discussed above, the conjugate preparations provided by the instant invention exhibit enhanced stability and substantial homogeneity due, at least in part, to the provision of engineered free cysteine site(s) and/or the novel conjugation procedures set forth herein. Unlike conventional conjugation methodology that fully or partially reduces each of the intrachain or interchain antibody disulfide bonds to provide conjugation sites, the present invention advantageously provides for the selective reduction of certain prepared free cysteine sites and direction of the drug-linker to the same. The conjugation specificity promoted by the engineered sites and attendant selective reduction allows for a high percentage of site directed conjugation at the desired positions. Significantly some of these conjugation sites, such as those present in the terminal region of the light chain constant region, are typically difficult to conjugate effectively as they cross-react with other free cysteines. However, through molecular engineering and selective reduction of the resulting free cysteines efficient conjugation rates may be obtained which considerably reduces unwanted high-DAR contaminants and non-specific toxicity. More generally the engineered constructs and disclosed novel conjugation methods comprising selective reduction apparently provide ADC preparations having improved pharmacokinetics and/or pharmacodynamics and, potentially, an improved therapeutic index.

In this respect the site-specific constructs present free cysteine(s), which when reduced comprise thiol groups that are nucleophilic and capable of reacting to form covalent bonds with electrophilic groups on linker moieties such as those disclosed immediately above. Preferred antibodies of the instant invention will have reducible unpaired interchain or intrachain cysteines, i.e. cysteines providing such nucleophilic groups. Thus, in certain embodiments the reaction of free sulfhydryl groups of the reduced unpaired cysteines and the terminal maleimido or haloacetamide groups of the disclosed drug-linkers will provide the desired conjugation. In such cases, and as set forth in Example 5 below, the free cysteines of the antibodies may be made reactive for conjugation with linker reagents by treatment with a reducing agent such as dithiothreitol (DTT) or (tris (2-carboxyethyl)phosphine (TCEP). Each free cysteine will thus present, theoretically, a reactive thiol nucleophile. While such reagents are compatible it will be appreciated that conjugation of the site-specific antibodies may be effected using various reactions, conditions and reagents known to those skilled in the art.

Conversely, the present inventors have discovered that the free cysteines of the engineered antibodies may be selectively reduced to provide enhanced site-directed conjugation and a reduction in unwanted, potentially toxic contaminants. More specifically “stabilizing agents” such as arginine have been found to modulate intra- and inter-molecular interactions in proteins and may be used, in conjunction with selected reducing agents (preferably relatively mild), to selectively reduce the free cysteines and to facilitate site-specific conjugation as set forth herein. As used herein the terms “selective reduction” or “selectively reducing” may be used interchangeably and shall mean the reduction of free cysteine(s) without substantially disrupting native disulfide bonds present in the engineered antibody. In selected embodiments this may be effected by certain reducing agents. In other preferred embodiments selective reduction of an engineered construct will comprise the use of stabilization agents in combination with reducing agents (including mild reducing agents). It will be appreciated that the term “selective conjugation” shall mean the conjugation of an engineered antibody that has been selectively reduced with a PBD as described herein. In this respect, and as demonstrated in Examples 6-8, the use of such stabilizing agents in combination with reducing agents can markedly improve the efficiency of site-specific conjugation as determined by extent of conjugation on the heavy and light antibody chains and DAR distribution of the preparation.

While not wishing to be bound by any particular theory, such stabilizing agents may act to modulate the electrostatic microenvironment and/or modulate conformational changes at the desired conjugation site, thereby allowing relatively mild reducing agents (which do not materially reduce intact native disulfide bonds) to facilitate conjugation at the desired free cysteine site. Such agents (e.g., certain amino acids) are known to form salt bridges (via hydrogen bonding and electrostatic interactions) and may modulate protein-protein interactions in such a way as to impart a stabilizing effect which may cause favorable conformation changes and/or may reduce unfavorable protein-protein interactions. Moreover, such agents may act to inhibit the formation of undesired intramolecular (and intermolecular) cysteine-cysteine bonds after reduction thus facilitating the desired conjugation reaction wherein the engineered site-specific cysteine is bound to the PBD (preferably via a linker). Since the reaction conditions do not provide for the significant reduction of intact native disulfide bonds the conjugation reaction is naturally driven to the relatively few reactive thiols on the free cysteines (e.g., preferably 2 free thiols). As alluded to this considerably reduces the levels of non-specific conjugation and corresponding impurities in conjugate preparations fabricated as set forth herein.

In selected embodiments stabilizing agents compatible with the present invention will generally comprise compounds with at least one amine moiety having a basic pKa. In certain embodiments the amine moiety will comprise a primary amine while in other preferred embodiments the amine moiety will comprise a secondary amine. In still other preferred embodiments the amine moiety will comprise a tertiary amine. In other selected embodiments the amine moiety will comprise an amino acid while in other compatible embodiments the amine moiety will comprise an amino acid side chain. In yet other embodiments the amine moiety will comprise a proteinogenic amino acid. In still other embodiments the amine moiety comprises a non-proteinogenic amino acid. In particularly preferred embodiments, compatible stabilizing agents may comprise arginine, lysine, proline and cysteine. In addition compatible stabilizing agents may include guanidine and nitrogen containing heterocycles with basic pKa.

In certain embodiments compatible stabilizing agents comprise compounds with at least one amine moiety having a pKa of greater than about 7.5, in other embodiments the subject amine moiety will have a pKa of greater than about 8.0, in yet other embodiments the amine moiety will have a pKa greater than about 8.5 and in still other embodiments the stabilizing agent will comprise an amine moiety having a pKa of greater than about 9.0. Other preferred embodiments will comprise stabilizing agents where the amine moiety will have a pKa of greater than about 9.5 while certain other embodiments will comprise stabilizing agents exhibiting at least one amine moiety having a pKa of greater than about 10.0. In still other preferred embodiments the stabilizing agent will comprise a compound having the amine moiety with a pKa of greater than about 10.5, in other embodiments the stabilizing agent will comprise a compound having a amine moiety with a pKa greater than about 11.0, while in still other embodiments the stabilizing agent will comprise a amine moiety with a pKa greater than about 11.5. In yet other embodiments the stabilizing agent will comprise a compound having an amine moiety with a pKa greater than about 12.0, while in still other embodiments the stabilizing agent will comprise an amine moiety with a pKa greater than about 12.5. Those of skill in the art will understand that relevant pKa's may readily be calculated or determined using standard techniques and used to determine the applicability of using a selected compound as a stabilizing agent.

The disclosed stabilizing agents are shown to be particularly effective at targeting conjugation to free site-specific cysteines when combined with certain reducing agents. For the purposes of the instant invention, compatible reducing agents may include any compound that produces a reduced free site-specific cysteine for conjugation without significantly disrupting the engineered antibody native disulfide bonds. Under such conditions, provided by the combination of selected stabilizing and reducing agents, the activated drug linker is largely limited to binding to the desired free site-specific cysteine site. Relatively mild reducing agents or reducing agents used at relatively low concentrations to provide mild conditions are particularly preferred. As used herein the terms “mild reducing agent” or “mild reducing conditions” shall be held to mean any agent or state brought about by a reducing agent (optionally in the presence of stabilizing agents) that provides thiols at the free cysteine site(s) without substantially disrupting native disulfide bonds present in the engineered antibody. That is, mild reducing agents or conditions are able to effectively reduce free cysteine(s) (provide a thiol) without significantly disrupting the protein's native disulfide bonds. The desired reducing conditions may be provided by a number of sulfhydryl-based compounds that establish the appropriate environment for selective conjugation. In preferred embodiments mild reducing agents may comprise compounds having one or more free thiols while in particularly preferred embodiments mild reducing agents will comprise compounds having a single free thiol. Non-limiting examples of reducing agents compatible with the instant invention comprise glutathione, n-acetyl cysteine, cysteine, 2-aminoethane-1-thiol and 2-hydroxyethane-1-thiol.

It will be appreciated that selective reduction process set forth above is particularly effective at targeted conjugation to the free cysteine. In this respect the extent of conjugation to the desired target site (defined here as “conjugation efficiency”) in site-specific antibodies may be determined by various art-accepted techniques. The efficiency of the site-specific conjugation of a PBD) to an antibody may be determined by assessing the percentage of conjugation on the target conjugation site (in this invention the free cysteine on the c-terminus of the light chain) relative to all other conjugated sites. In certain embodiments, the method herein provides for efficiently conjugating a PBD to an antibody comprising free cysteines. In some embodiments, the conjugation efficiency is at least 5%, at least 10%, at least 15%, at least 20%, at least 25%, at least 30%, at least 35%, at least 40%, at least 45%, at least 45%, at least 50%, at least 55%, at least 60%, at least 70%, at least 75%, at least 80%, at least 85%, at least 90%, at least 95%, at least 98% or more as measured by the percentage of target conjugation relative to all other conjugation sites.

It will further be appreciated that the engineered antibodies capable of conjugation may contain free cysteine residues that comprise sulfhydryl groups that are blocked or capped as the antibody is produced or stored. Such caps include proteins, peptides, ions and other materials that interact with the sulfhydryl group and prevent or inhibit conjugate formation. In some cases the unconjugated engineered antibody may comprise free cysteines that bind other free cysteines on the same or different antibodies. As discussed in the Examples such cross-reactivity may lead to various contaminants during the fabrication procedure. In some embodiments, the engineered antibodies may require uncapping prior to a conjugation reaction. In specific embodiments, antibodies herein are uncapped and display a free sulfhydryl group capable of conjugation. In specific embodiments, antibodies herein are subjected to an uncapping reaction that does not disturb or rearrange the naturally occurring disulfide bonds. It will be appreciated that in most cases the uncapping reactions will occur during the normal reduction reactions (reduction or selective reduction).

4. DAR Distribution and Purification

One of the advantages of the present invention is the ability to generate relatively homogeneous conjugate preparations comprising a narrowly tailored DAR distribution. In this regard the disclosed constructs and/or selective conjugation provides for homogeneity of the ADC species within a sample in terms of the stoichiometric ratio between the PBD and the engineered antibody. As briefly discussed above the term “drug to antibody ratio” or “DAR” refers to the molar ratio of PBD to site-specific antibody. In some embodiments a conjugate preparation may be substantially homogeneous with respect to its DAR distribution, meaning that within the preparation is a predominant species of site-specific ADC with a particular DAR (e.g., a DAR of 2 or 4) that is also uniform with respect to the site of loading (i.e., on the free cysteines). In certain embodiments of the invention it is possible to achieve the desired homogeneity through the use of site-specific antibodies or selective combination. In other preferred embodiments the desired homogeneity may be achieved through the use of site-specific constructs in combination with selective reduction. In yet other particularly preferred embodiments the preparations may be further purified using analytical or preparative chromatography techniques. In each of these embodiments the homogeneity of the ADC sample can be analyzed using various techniques known in the art including but not limited to SDS-PAGE, HPLC (e.g. size exclusion HPLC, RP-HPLC, HIC-HPLC etc.) or capillary electrophoresis.

With regard to the purification of ADC preparations it will be appreciated that standard pharmaceutical preparative methods may be employed to obtain the desired purity. As demonstrated in the Examples below liquid chromatography methods such as reverse phase (RP) and hydrophobic interaction chromatography (HIC) may separate compounds in the mixture by drug loading value. In some cases, mixed-mode chromatography (MMC) may also be used to isolate species with a specific drug load. More generally, once insoluble contaminants are removed the modulator preparation may be further purified using standard techniques such as, for example, hydroxylapatite chromatography, gel electrophoresis, dialysis, and affinity chromatography, with affinity chromatography of particular interest. In this regard protein A can be used to purify antibodies that are based on human IgG1, IgG2 or IgG4 heavy chains while protein G is recommended for all mouse isotypes and for human IgG3. Other techniques for protein purification such as fractionation on an ion-exchange column, ethanol precipitation, chromatography on silica, chromatography on heparin, sepharose chromatography on an anion or cation exchange resin (such as a polyaspartic acid column), chromatofocusing, SDS-PAGE and ammonium sulfate precipitation are also available depending on the antibody or conjugate to be recovered.

In this regard the disclosed site-specific conjugates and preparations thereof may comprise drug and antibody moieties in various stoichiometric molar ratios depending on the configuration of the engineered construct and, at least in part, on the method used to effect conjugation. Depending on how many and which interchain and intrachain disulfide bonds are disrupted theoretical drug loading may be relatively high though practical limitations such as free cysteine cross reactivity would limit the generation of homogeneous preparations comprising such DAR due to aggregates and other contaminants. That is, higher drug loading, e.g. >6, may cause aggregation, insolubility, toxicity, or loss of cellular permeability of certain antibody-drug conjugates. In view of such concerns practical drug loading provided by the instant invention may range from 1 to 8 drugs per engineered conjugate, i.e. where 1, 2, 3, 4, 5, 6, 7, or 8 PBDs are covalently attached to each site specific antibody (e.g., for IgG1, other antibodies may have different loading capacity depending the number of disulfide bonds). Preferably the DAR of compositions of the instant invention will be approximately 2, 4 or 6 and in particularly preferred embodiments the DAR will comprise approximately 2.

Despite the relatively high level of homogeneity provided by the instant invention the disclosed compositions actually comprise a mixture engineered conjugates with a range of PBD compounds, from 1 to 8 (in the case of a IgG1). As such, the disclosed ADC compositions include mixtures of conjugates where most of the constituent antibodies are covalently linked to one or more PBD drug moieties and (despite the conjugate specificity of selective reduction) where the drug moieties may be attached to the antibody by various thiol groups. That is, following conjugation ADC compositions of the invention will comprise a mixture of anti-DLL3 conjugates with different drug loads (e.g., from 1 to 8 drugs per IgG1 antibody) at various concentrations (along with certain reaction contaminants primarily caused by free cysteine cross reactivity). Using selective reduction and post-fabrication purification the conjugate compositions may be driven to the point where they largely contain a single predominant desired ADC species (e.g., with a drug loading of 2) with relatively low levels of other ADC species (e.g., with a drug loading of 1, 4, 6, etc.). The average DAR value represents the weighted average of drug loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting.

Thus, in certain preferred embodiments the present invention will comprise compositions having an average DAR of 1, 2, 3, 4, 5, 6, 7 or 8 each +/−0.5. In other preferred embodiments the present invention will comprise an average DAR of 2, 4, 6 or 8+/−0.5. Finally, in selected preferred embodiments the present invention will comprise an average DAR of 2+/−0.5. It will be appreciated that the range or deviation may be less than 0.4 in certain preferred embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4 and relatively low levels (i.e., less than 30%) of non-predominant ADC species. In other preferred embodiments the ADC composition will comprise an average DAR of 2, 4, 6 or 8 each +/−0.4 with relatively low levels (<30%) of non-predominant ADC species. In particularly preferred embodiments the ADC composition will comprise an average DAR of 2+/−0.4 with relatively low levels (<30%) of non-predominant ADC species. In yet other embodiments the predominant ADC species (e.g., DAR of 2) will be present at a concentration of greater than 70%, a concentration of greater than 75%, a concentration of greater that 80%, a concentration of greater than 85%, a concentration of greater than 90%, a concentration of greater than 93%, a concentration of greater than 95% or even a concentration of greater than 97% when measured against other DAR species.

As detailed in the Examples below the distribution of drugs per antibody in preparations of ADC from conjugation reactions may be characterized by conventional means such as UV-Vis spectrophotometry, reverse phase HPLC, HIC, mass spectroscopy, ELISA, and electrophoresis. The quantitative distribution of ADC in terms of drugs per antibody may also be determined. By ELISA, the averaged value of the drugs per antibody in a particular preparation of ADC may be determined. However, the distribution of drug per antibody values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues.

### V. Pharmaceutical Preparations and Therapeutic Uses

1. Formulations and Routes of Administration

Depending on the form of the selected site-specific conjugate, the mode of intended delivery, the disease being treated or monitored and numerous other variables, compositions of the invention may be formulated as desired using art-recognized techniques. In some embodiments, the therapeutic compositions of the invention may be administered neat or with a minimum of additional components while others may optionally be formulated to contain suitable pharmaceutically acceptable carriers comprising excipients and auxiliaries that are well known in the art (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents and the like, are also available. Certain non-limiting exemplary carriers include saline, buffered saline, dextrose, water, glycerol, ethanol, and combinations thereof.

More particularly it will be appreciated that, in some embodiments, the therapeutic compositions of the invention may be administered neat or with a minimum of additional components. Conversely the anti-DLL3 site-specific ADCs of the present invention may optionally be formulated to contain suitable pharmaceutically acceptable carriers comprising excipients and auxiliaries that are well known in the art and are relatively inert substances that facilitate administration of the conjugate or which aid processing of the active compounds into preparations that are pharmaceutically optimized for delivery to the site of action. For example, an excipient can give form or consistency or act as a diluent to improve the pharmacokinetics or stability of the ADC. Suitable excipients or additives include, but are not limited to, stabilizing agents, wetting and emulsifying agents, salts for varying osmolarity, encapsulating agents, buffers, and skin penetration enhancers. In certain preferred embodiments the pharmaceutical compositions may be provided in a lyophilized form and reconstituted in, for example, buffered saline prior to administration. Such reconstituted compositions are preferably administered intravenously.

Disclosed ADCs for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington, The Science and Practice of Pharmacy 20th Ed. Mack Publishing (2000). Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, hexylsubstituted poly(lactide), sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection.

Compatible formulations for parenteral administration (e.g., intravenous injection) will comprise ADC concentrations of from about 10 μg/ml to about 100 mg/ml. In certain selected embodiments ADC concentrations will comprise 20 μg/ml, 40 μg/ml, 60 μg/ml, 80 μg/ml, 100 jag/ml, 200 μg/ml, 300, jag/ml, 400 μg/ml, 500 μg/ml, 600 μg/ml, 700 μg/ml, 800 μg/ml, 900 μg/ml or 1 mg/ml. In other preferred embodiments ADC concentrations will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

In general the compounds and compositions of the invention, comprising anti-DLL3 site-specific ADCs may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen. In particularly preferred embodiments the compounds of the instant invention will be delivered intravenously.

2. Dosages

Similarly, the particular dosage regimen, i.e., dose, timing and repetition, will depend on the particular individual and that individual's medical history, as well as empirical considerations such as pharmacokinetics (e.g., half-life, clearance rate, etc.). Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In other embodiments the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages of the conjugate compound, and compositions comprising the conjugate compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the site-specific ADCs of the invention may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In selected embodiments the site-specific ADCs will be administered (preferably intravenously) at approximately 10, 20, 30, 40, 50, 60, 70, 80, 90 or 100 μg/kg body weight per dose. Other embodiments will comprise the administration of ADCs at about 200, 300, 400, 500, 600, 700, 800, 900, 1000, 1100, 1200, 1300, 1400, 1500, 1600, 1700, 1800, 1900 or 2000 μg/kg body weight per dose. In other preferred embodiments the disclosed conjugates will be administered at 2.5, 3, 3.5, 4, 4.5, 5, 5.5, 6, 6.5, 7, 7.58, 9 or 10 mg/kg. In still other embodiments the conjugates may be administered at 12, 14, 16, 18 or 20 mg/kg body weight per dose. In yet other embodiments the conjugates may be administered at 25, 30, 35, 40, 45, 50, 55, 60, 65, 70, 75, 80, 90 or 100 mg/kg body weight per dose. With the teachings herein one of skill in the art could readily determine appropriate dosages for various site-specific ADCs based on preclinical animal studies, clinical observations and standard medical and biochemical techniques and measurements. In particularly preferred embodiments such DLL3 conjugate dosages will be administered intravenously over a period of time. Moreover, such dosages may be administered multiple times over a defined course of treatment.

Other dosing regimens may be predicated on Body Surface Area (BSA) calculations as disclosed in U.S. Pat. No. 7,744,877. As is well known, the BSA is calculated using the patient's height and weight and provides a measure of a subject's size as represented by the surface area of his or her body. In certain embodiments, the conjugates may be administered in dosages from 1 mg/m² to 800 mg/m², from 50 mg/m² to 500 mg/m² and at dosages of 100 mg/m², 150 mg/m², 200 mg/m², 250 mg/m², 300 mg/m², 350 mg/m², 400 mg/m² or 450 mg/m². It will also be appreciated that art recognized and empirical techniques may be used to determine appropriate dosage.

In any event, DLL3 ADCs are preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like. Generally, an effective dose of the DLL3 conjugate is administered to a subject one or more times. More particularly, an effective dose of the ADC is administered to the subject once a month, more than once a month, or less than once a month. In certain embodiments, the effective dose of the DLL3 ADC may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the disclosed modulators.

In certain preferred embodiments the course of treatment involving conjugated modulators will comprise multiple doses of the selected drug product over a period of weeks or months. More specifically, conjugated modulators of the instant invention may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

3. Combination Therapies

In accordance with the instant invention combination therapies may be particularly useful in decreasing or inhibiting unwanted neoplastic cell proliferation, decreasing the occurrence of cancer, decreasing or preventing the recurrence of cancer, or decreasing or preventing the spread or metastasis of cancer. In such cases the ADCs of the instant invention may function as sensitizing or chemosensitizing agents by removing the CSCs that would otherwise prop up and perpetuate the tumor mass and thereby allow for more effective use of current standard of care debulking or anti-cancer agents. That is, the disclosed ADCs may, in certain embodiments provide an enhanced effect (e.g., additive or synergistic in nature) that potentiates the mode of action of another administered therapeutic agent. In the context of the instant invention “combination therapy” shall be interpreted broadly and merely refers to the administration of an anti-DLL3 site-specific ADC and one or more anti-cancer agents that include, but are not limited to, cytotoxic agents, cytostatic agents, anti-angiogenic agents, debulking agents, chemotherapeutic agents, radiotherapy and radiotherapeutic agents, targeted anti-cancer agents (including both monoclonal antibodies and small molecule entities), BRMs, therapeutic antibodies, cancer vaccines, cytokines, hormone therapies, radiation therapy and anti-metastatic agents and immunotherapeutic agents, including both specific and non-specific approaches.

There is no requirement for the combined results to be additive of the effects observed when each treatment (e.g., ADC and anti-cancer agent) is conducted separately. Although at least additive effects are generally desirable, any increased anti-tumor effect above one of the single therapies is beneficial. Furthermore, the invention does not require the combined treatment to exhibit synergistic effects. However, those skilled in the art will appreciate that with certain selected combinations that comprise preferred embodiments, synergism may be observed.

In practicing combination therapy, the DLL3 conjugate and anti-cancer agent may be administered to the subject simultaneously, either in a single composition, or as two or more distinct compositions using the same or different administration routes. Alternatively, the ADC may precede, or follow, the anti-cancer agent treatment by, e.g., intervals ranging from minutes to weeks. The time period between each delivery is such that the anti-cancer agent and conjugate are able to exert a combined effect on the tumor. In at least one embodiment, both the anti-cancer agent and the ADC are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the DLL3 ADC and the anti-cancer agent.

The combination therapy may be administered once, twice or at least for a period of time until the condition is treated, palliated or cured. In some embodiments, the combination therapy is administered multiple times, for example, from three times daily to once every six months. The administering may be on a schedule such as three times daily, twice daily, once daily, once every two days, once every three days, once weekly, once every two weeks, once every month, once every two months, once every three months, once every six months or may be administered continuously via a minipump. The combination therapy may be administered via any route, as noted previously. The combination therapy may be administered at a site distant from the site of the tumor.

In one embodiment a site-specific ADC is administered in combination with one or more anti-cancer agents for a short treatment cycle to a subject in need thereof. The invention also contemplates discontinuous administration or daily doses divided into several partial administrations. The conjugate and anti-cancer agent may be administered interchangeably, on alternate days or weeks; or a sequence of antibody treatments may be given, followed by one or more treatments of anti-cancer agent therapy. In any event, as will be understood by those of ordinary skill in the art, the appropriate doses of chemotherapeutic agents and the disclosed conjugates will be generally around those already employed in clinical therapies wherein the chemotherapeutics are administered alone or in combination with other chemotherapeutics.

In another preferred embodiment the DLL3 conjugates of the instant invention may be used in maintenance therapy to reduce or eliminate the chance of tumor recurrence following the initial presentation of the disease. Preferably the disorder will have been treated and the initial tumor mass eliminated, reduced or otherwise ameliorated so the patient is asymptomatic or in remission. At such time the subject may be administered pharmaceutically effective amounts of the disclosed DLL3 conjugates one or more times even though there is little or no indication of disease using standard diagnostic procedures. In some embodiments, the ADCs will be administered on a regular schedule over a period of time, such as weekly, every two weeks, monthly, every six weeks, every two months, every three months every six months or annually. Given the teachings herein, one skilled in the art could readily determine favorable dosages and dosing regimens to reduce the potential of disease recurrence. Moreover such treatments could be continued for a period of weeks, months, years or even indefinitely depending on the patient response and clinical and diagnostic parameters.

In yet another preferred embodiment the ADCs of the present invention may be used to prophylactically or as an adjuvant therapy to prevent or reduce the possibility of tumor metastasis following a debulking procedure. As used in the instant disclosure a “debulking procedure” is defined broadly and shall mean any procedure, technique or method that eliminates, reduces, treats or ameliorates a tumor or tumor proliferation. Exemplary debulking procedures include, but are not limited to, surgery, radiation treatments (i.e., beam radiation), chemotherapy, immunotherapy or ablation. At appropriate times readily determined by one skilled in the art in view of the instant disclosure the disclosed ADCs may be administered as suggested by clinical, diagnostic or theragnostic procedures to reduce tumor metastasis. The conjugates may be administered one or more times at pharmaceutically effective dosages as determined using standard techniques. Preferably the dosing regimen will be accompanied by appropriate diagnostic or monitoring techniques that allow it to be modified.

Yet other embodiments of the invention comprise administering the disclosed DLL3 conjugates to subjects that are asymptomatic but at risk of developing a proliferative disorder. That is, the conjugates of the instant invention may be used in a truly preventative sense and given to patients that have been examined or tested and have one or more noted risk factors (e.g., genomic indications, family history, in vivo or in vitro test results, etc.) but have not developed neoplasia. In such cases those skilled in the art would be able to determine an effective dosing regimen through empirical observation or through accepted clinical practices.

4. Anti-Cancer Agents

As discussed throughout the instant application the anti-DLL3 site-specific conjugates of the instant invention may be used in combination with anti-cancer agents. The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents.

As used herein the term “cytotoxic agent” means a substance that is toxic to the cells and decreases or inhibits the function of cells and/or causes destruction of cells. In certain embodiments the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the instant invention a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the DLL3 ADCs of the present invention include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Particularly preferred anti-cancer agents comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, W O 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

In other embodiments the DLL3 conjugates of the instant invention may be used in combination with any one of a number of antibodies (or immunotherapeutic agents) presently in clinical trials or commercially available. To this end the disclosed DLL3 conjugates may be used in combination with an antibody selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, ramucirumab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8 and combinations thereof.

Still other particularly preferred embodiments will comprise the use of antibodies in testing or approved for cancer therapy including, but not limited to, rituximab, trastuzumab, gemtuzumab ozogamcin, alemtuzumab, ibritumomab tiuxetan, tositumomab, bevacizumab, cetuximab, panitumumab, ramucirumab, ofatumumab, ipilimumab and brentuximab vedotin. Those skilled in the art will be able to readily identify additional anti-cancer agents that are compatible with the teachings herein.

5. Radiotherapy

The present invention also provides for the combination of DLL3 conjugates with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### VI. Indications

It will be appreciated that the ADCs of the instant invention may be used to treat, prevent, manage or inhibit the occurrence or recurrence of any DLL3 associated disorder. Accordingly, whether administered alone or in combination with an anti-cancer agent or radiotherapy, the ADCs of the invention are particularly useful for generally treating neoplastic conditions in patients or subjects which may include benign or malignant tumors (e.g., adrenal, liver, kidney, bladder, breast, gastric, ovarian, colorectal, prostate, pancreatic, lung, thyroid, hepatic, cervical, endometrial, esophageal and uterine carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders; and inflammatory, angiogenic, immunologic disorders and disorders caused by pathogens. Particularly, key targets for treatment are neoplastic conditions comprising solid tumors, although hematologic malignancies are within the scope of the invention.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

More specifically, neoplastic conditions subject to treatment in accordance with the instant invention may be selected from the group including, but not limited to, adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma).

In certain preferred embodiments the proliferative disorder will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors. In other preferred embodiments, and as shown in the Examples below, the disclosed ADCs are especially effective at treating small cell lung cancer (SCLC) and non-small cell lung cancer (NSCLC) (e.g., squamous cell non-small cell lung cancer or squamous cell small cell lung cancer). In one embodiment, the lung cancer is refractory, relapsed or resistant to a platinum based agent (e.g., carboplatin, cisplatin, oxaliplatin, topotecan) and/or a taxane (e.g., docetaxel, paclitaxel, larotaxel or cabazitaxel).

In particularly preferred embodiments the disclosed ADCs may be used to treat small cell lung cancer. With regard to such embodiments the conjugated modulators may be administered to patients exhibiting limited stage disease. In other embodiments the disclosed ADCs will be administered to patients exhibiting extensive stage disease. In other preferred embodiments the disclosed ADCs will be administered to refractory patients (i.e., those who recur during or shortly after completing a course of initial therapy) or recurrent small cell lung cancer patients. Still other embodiments comprise the administration of the disclosed ADCs to sensitive patients (i.e., those whose relapse is longer than 2-3 months after primary therapy. In each case it will be appreciated that compatible ADCs may be used in combination with other anti-cancer agents depending the selected dosing regimen and the clinical diagnosis.

As discussed above the disclosed ADCs may further be used to prevent, treat or diagnose tumors with neuroendocrine features or phenotypes including neuroendocrine tumors. True or canonical neuroendocrine tumors (NETs) arising from the dispersed endocrine system are relatively rare, with an incidence of 2-5 per 100,000 people, but highly aggressive. Neuroendocrine tumors occur in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). These tumors may secrete several hormones including serotonin and/or chromogranin A that can cause debilitating symptoms known as carcinoid syndrome. Such tumors can be denoted by positive immunohistochemical markers such as neuron-specific enolase (NSE, also known as gamma enolase, gene symbol=ENO2), CD56 (or NCAM1), chromogranin A (CHGA), and synaptophysin (SYP) or by genes known to exhibit elevated expression such as ASCL1. Unfortunately traditional chemotherapies have not been particularly effective in treating NETs and liver metastasis is a common outcome.

While the disclosed ADCs may be advantageously used to treat neuroendocrine tumors they may also be used to treat, prevent or diagnose pseudo neuroendocrine tumors (pNETs) that genotypically or phenotypically mimic, resemble or exhibit common traits with canonical neuroendocrine tumors. Pseudo neuroendocrine tumors or tumors with neuroendocrine features are tumors that arise from cells of the diffuse neuroendocrine system or from cells in which a neuroendocrine differentiation cascade has been aberrantly reactivated during the oncogenic process. Such pNETs commonly share certain phenotypic or biochemical characteristics with traditionally defined neuroendocrine tumors, including the ability to produce subsets of biologically active amines, neurotransmitters, and peptide hormones. Histologically, such tumors (NETs and pNETs) share a common appearance often showing densely connected small cells with minimal cytoplasm of bland cytopathology and round to oval stippled nuclei. For the purposes of the instant invention commonly expressed histological markers or genetic markers that may be used to define neuroendocrine and pseudo neuroendocrine tumors include, but are not limited to, chromogranin A, CD56, synaptophysin, PGP9.5, ASCL1 and neuron-specific enolase (NSE).

Accordingly the ADCs of the instant invention may beneficially be used to treat both pseudo neuroendocrine tumors and canonical neuroendocrine tumors. In this regard the ADCs may be used as described herein to treat neuroendocrine tumors (both NET and pNET) arising in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). Moreover, the ADCs of the instant invention may be used to treat tumors expressing one or more markers selected from the group consisting of NSE, CD56, synaptophysin, chromogranin A, ASCL1 and PGP9.5 (UCHL1). That is, the present invention may be used to treat a subject suffering from a tumor that is NSE⁺ or CD56⁺ or PGP9.5⁺ or ASCL1⁺ or SYP⁺ or CHGA⁺ or some combination thereof.

With regard to hematologic malignancies it will be further be appreciated that the compounds and methods of the present invention may be particularly effective in treating a variety of B-cell lymphomas, including low grade/NHL follicular cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, Waldenstrom's Macroglobulinemia, lymphoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic, follicular, diffuse large cell, diffuse small cleaved cell, large cell immunoblastic lymphoblastoma, small, non-cleaved, Burkitt's and non-Burkitt's, follicular, predominantly large cell; follicular, predominantly small cleaved cell; and follicular, mixed small cleaved and large cell lymphomas. See, Gaidono et al., “Lymphomas”, IN CANCER: PRINCIPLES & PRACTICE OF ONCOLOGY, Vol. 2: 2131-2145 (DeVita et al., eds., 5.sup.th ed. 1997). It should be clear to those of skill in the art that these lymphomas will often have different names due to changing systems of classification, and that patients having lymphomas classified under different names may also benefit from the combined therapeutic regimens of the present invention.

The present invention also provides for a preventative or prophylactic treatment of subjects who present with benign or precancerous tumors. Beyond being a DLL3 associated disorder it is not believed that any particular type of tumor or proliferative disorder should be excluded from treatment using the present invention. However, the type of tumor cells may be relevant to the use of the invention in combination with secondary therapeutic agents, particularly chemotherapeutic agents and targeted anti-cancer agents.

Preferably the “subject” or “patient” to be treated will be human although, as used herein, the terms are expressly held to comprise any species including all mammals. Accordingly the subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

### VII. Articles of Manufacture

Pharmaceutical packs and kits comprising one or more containers, comprising one or more doses of an anti-DLL3 site-specific ADC are also provided. In certain embodiments, a unit dosage is provided wherein the unit dosage contains a predetermined amount of a composition comprising, for example, an anti-DLL3 conjugate, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present invention also provides kits for producing single-dose or multi-dose administration units of a DLL3 conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed DLL3 conjugates in a conjugated or unconjugated form. In other preferred embodiments the container(s) comprise a sterile access port (for example the container may be an intravenous solution bag or a vial having a stopper pierceable by a hypodermic injection needle). Such kits will generally contain in a suitable container a pharmaceutically acceptable formulation of the DLL3 conjugate and, optionally, one or more anti-cancer agents in the same or different containers. The kits may also contain other pharmaceutically acceptable formulations, either for diagnosis or combined therapy. For example, in addition to the DLL3 conjugates of the invention such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the DLL3 ADCs, with or without additional components, or they may have distinct containers for each desired agent. Where combined therapeutics are provided for conjugation, a single solution may be pre-mixed, either in a molar equivalent combination, or with one component in excess of the other. Alternatively, the DLL3 conjugates and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody conjugate and any optional components to an animal or patient, e.g., one or more needles, I.V. bags or syringes, or even an eye dropper, pipette, or other such like apparatus, from which the formulation may be injected or introduced into the animal or applied to a diseased area of the body. The kits of the present invention will also typically include a means for containing the vials, or such like, and other component in close confinement for commercial sale, such as, e.g., injection or blow-molded plastic containers into which the desired vials and other apparatus are placed and retained. Any label or package insert indicates that the DLL3 conjugate composition is used for treating cancer, for example small cell lung cancer.

In other preferred embodiments the conjugates of the instant invention may be used in conjunction with, or comprise, diagnostic or therapeutic devices useful in the prevention or treatment of proliferative disorders. For example, in on preferred embodiment the compounds and compositions of the instant invention may be combined with certain diagnostic devices or instruments that may be used to detect, monitor, quantify or profile cells or marker compounds involved in the etiology or manifestation of proliferative disorders. For selected embodiments the marker compounds may comprise NSE, CD56, synaptophysin, chromogranin A, and PGP9.5.

In particularly preferred embodiments the devices may be used to detect, monitor and/or quantify circulating tumor cells either in vivo or in vitro (see, for example, WO 2012/0128801 which is incorporated herein by reference). In still other preferred embodiments, and as discussed above, circulating tumor cells may comprise cancer stem cells.

### VIII. Miscellaneous

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In addition, ranges provided in the specification and appended claims include both end points and all points between the end points. Therefore, a range of 2.0 to 3.0 includes 2.0, 3.0, and all points between 2.0 and 3.0.

Generally, nomenclature used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well known and commonly used in the art. Moreover, any section headings used herein are for organizational purposes only and are not to be construed as limiting the subject matter described.

As used herein, tumor cell types are abbreviated as follows: adenocarcinoma (Adeno), adrenal (AD), breast (BR), estrogen receptor positive breast (BR-ER+), estrogen receptor negative breast (BR-ER−), progesterone receptor positive breast (BR-PR+), progesterone receptor negative breast (BR-PR−), ERb2/Neu positive breast (BR-ERB2/Neu+), Her2 positive breast (BR-Her2+), claudin-low breast (BR-CLDN-lo), triple-negative breast cancer (BR-TNBC), colorectal (CR), endometrial (EM), gastric (GA), head and neck (HN), kidney (KDY), large cell neuroendocrine (LCNEC), liver (LIV), lymph node (LN), lung (LU), lung-carcinoid (LU-CAR), lung-spindle cell (LU-SPC), melanoma (MEL), non-small cell lung (NSCLC), ovarian (OV), ovarian serous (OV-S), ovarian papillary serous (OV-PS), ovarian malignant mixed mesodermal tumor (OV-MMMT), ovarian mucinous (OV-MUC), ovarian clear cell (OV-CC), neuroendocrine tumor (NET), pancreatic (PA), prostate (PR), squamous cell (SCC), small cell lung (SCLC) and tumors derived from skin (SK).

### IX. References

Unless The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PBD, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described.

### X. Sequence Listing Summary

Appended to the instant application is a sequence listing comprising a number of nucleic acid and amino acid sequences. The following TABLE 4 provides a summary of the included sequences.

TABLE 4

| SEQ ID NO. | Description |
|---|---|
| 1 | DLL3 isoform 1 protein |
| 2 | DLL3 isoform 2 protein |
| 3 | Epitope SC16.23 protein |
| 4 | Epitope SC16.34 & SC 16.56 protein |
| 5 | Kappa light chain constant region protein |
| 6 | IgG1 heavy chain constant region protein |
| 7 | C220S IgG1 heavy constant region protein |
| 8 | C220Δ IgG1 heavy constant region protein |
| 9 | C214Δ Kappa light chain constant region protein |
| 10 | C214S Kappa light chain constant region protein |
| 11 | Lambda light chain constant region protein |
| 12 | C214Δ Lambda light chain constant region protein |
| 13 | C214S Lambda light chain constant region protein |
| 14 | SC16.56 ss1 and ss2 full length light chain protein |
| 15 | SC16.56 ss3 and ss4 full length heavy chain protein |
| 16 | SC16.56 ss1 full length heavy chain protein |
| 17 | SC16.56 ss2 full length heavy chain protein |
| 18 | SC16.56 ss3 full length light chain protein |
| 19 | SC16.56 ss4 full length light chain protein |
| 20 | SC16.3 VL DNA (aligned with encoded protein) |
| 21 | SC16.3 VL protein |
| 22 | SC16.3 VH DNA (aligned with encoded protein) |
| 23 | SC16.3 VH protein |
| 24-387 | Additional murine clones as in SEQ ID NOs: 20-23 |
| 388-407 | Humanized clones as in SEQ ID NOs: 20-23 |
| 408, 409, 410 | hSC16.13 CDRL1, CDRL2, CDRL3 |
| 411, 412, 413 | hSC16.13 CDRH1, CDRH2, CDRH3 |
| 414, 415, 416 | hSC16.15 CDRL1, CDRL2, CDRL3 |
| 417, 418, 419 | hSC16.15 CDRH1, CDRH2, CDRH3 |
| 420, 421, 422 | hSC16.25 CDRL1, CDRL2, CDRL3 |
| 423, 424, 425 | hSC16.25 CDRH1, CDRH2, CDRH3 |
| 426, 427, 428 | hSC16.34 CDRL1, CDRL2, CDRL3 |
| 429, 430, 431 | hSC16.34 CDRH1, CDRH2, CDRH3 |
| 432, 433, 434 | hSC16.56 CDRL1, CDRL2, CDRL3 |
| 435, 436, 437 | hSC16.56 CDRH1, CDRH2, CDRH3 |

### EXAMPLES

The present invention, thus generally described, will be understood more readily by reference to the following Examples, which are provided by way of illustration and are not intended to be limiting of the instant invention. The Examples are not intended to represent that the experiments below are all or the only experiments performed.

### Example 1

### Generation of Anti-DLL3 Antibodies

Anti-DLL3 murine antibodies were produced as follows. In a first immunization campaign, three mice (one from each of the following strains: Balb/c, CD-1, FVB) were inoculated with human DLL3-fc protein (hDLL3-Fc) emulsified with an equal volume of TiterMax® or alum adjuvant. The hDLL3-Fc fusion construct was purchased from Adipogen International (Catalog No. AG-40A-0113). An initial immunization was performed with an emulsion of 10 μg hDLL3-Fc per mouse in TiterMax. Mice were then boosted biweekly with 5 μg hDLL3-Fc per mouse in alum adjuvant. The final injection prior to fusion was with 5 μg hDLL3-Fc per mouse in PBS.

In a second immunization campaign six mice (two each of the following strains: Balb/c, CD-1, FVB), were inoculated with human DLL3-His protein (hDLL3-His), emulsified with an equal volume of TiterMax® or alum adjuvant. Recombinant hDLL3-His protein was purified from the supernatants of CHO—S cells engineered to overexpress hDLL3-His. The initial immunization was with an emulsion of 10 μg hDLL3-His per mouse in TiterMax. Mice were then boosted biweekly with 5 μg hDLL3-His per mouse in alum adjuvant. The final injection was with 2×10⁵ HEK-293T cells engineered to overexpress hDLL3.

Solid-phase ELISA assays were used to screen mouse sera for mouse IgG antibodies specific for human DLL3. A positive signal above background was indicative of antibodies specific for DLL3. Briefly, 96 well plates (VWR International, Cat. #610744) were coated with recombinant DLL3-His at 0.5 μg/ml in ELISA coating buffer overnight. After washing with PBS containing 0.02% (v/v) Tween 20, the wells were blocked with 3% (w/v) BSA in PBS, 200 μL/well for 1 hour at room temperature (RT). Mouse serum was titrated (1:100, 1:200, 1:400, and 1:800) and added to the DLL3 coated plates at 50 μL/well and incubated at RT for 1 hour. The plates are washed and then incubated with 50 μL/well HRP-labeled goat anti-mouse IgG diluted 1:10,000 in 3% BSA-PBS or 2% FCS in PBS for 1 hour at RT. Again the plates were washed and 40 μL/well of a TMB substrate solution (Thermo Scientific 34028) was added for 15 minutes at RT. After developing, an equal volume of 2N H₂SO₄ was added to stop substrate development and the plates were analyzed by spectrophotometer at OD 450.

Sera-positive immunized mice were sacrificed and draining lymph nodes (popliteal, inguinal, and medial iliac) were dissected and used as a source for antibody producing cells. Cell suspensions of B cells (approximately 229×10⁶ cells from the hDLL3-Fc immunized mice, and 510×10⁶ cells from the hDLL3-His immunized mice) were fused with non-secreting P3×63Ag8.653 myeloma cells at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum, 10% BM Condimed (Roche Applied Sciences), 1 mM nonessential amino acids, 1 mM HEPES, 100 IU penicillin-streptomycin, and 50 μM 2-mercaptoethanol, and were cultured in four T225 flasks in 100 mL selection medium per flask. The flasks were placed in a humidified 37° C. incubator containing 5% CO₂ and 95% air for six to seven days.

On day six or seven after the fusions the hybridoma library cells were collected from the flasks and plated at one cell per well (using the FACSAria I cell sorter) in 200 μL of supplemented hybridoma selection medium (as described above) into 64 Falcon 96-well plates, and 48 96-well plates for the hDLL3-His immunization campaign. The rest of the library was stored in liquid nitrogen.

The hybridomas were cultured for 10 days and the supernatants were screened for antibodies specific to hDLL3 using flow cytometry performed as follows. 1×10⁵ per well of HEK-293T cells engineered to overexpress human DLL3, mouse DLL3 (pre-stained with dye), or cynomolgus DLL3 (pre-stained with Dylight800) were incubated for 30 minutes with 25 μL hybridoma supernatant. Cells were washed with PBS/2% FCS and then incubated with 25 μL per sample DyeLight 649 labeled goat-anti-mouse IgG, Fc fragment specific secondary diluted 1:300 in PBS/2% FCS. After a 15 minute incubation cells were washed twice with PBS/2% FCS and re-suspended in PBS/2% FCS with DAPI and analyzed by flow cytometry for fluorescence exceeding that of cells stained with an isotype control antibody. Remaining unused hybridoma library cells were frozen in liquid nitrogen for future library testing and screening.

The hDLL3-His immunization campaign yielded approximately 50 murine anti-hDLL3 antibodies and the hDLL3-Fc immunization campaign yielded approximately 90 murine anti-hDLL3 antibodies.

### Example 2

### Sequencing of Anti-DLL3 Antibodies

Based on the foregoing, a number of exemplary distinct monoclonal antibodies that bind immobilized human DLL3 or h293-hDLL3 cells with apparently high affinity were selected for sequencing and further analysis. Sequence analysis of the light chain variable regions and heavy chain variable regions from selected monoclonal antibodies generated in Example 1 confirmed that many had novel complementarity determining regions and often displayed novel VDJ arrangements.

Initially selected hybridoma cells expressing the desired antibodies were lysed in Trizol® reagent (Trizol® Plus RNA Purification System, Life Technologies) to prepare the RNA encoding the antibodies. Between 10⁴ and 10⁵ cells were re-suspended in 1 mL Trizol and shaken vigorously after addition of 200 μL chloroform. Samples were then centrifuged at 4° C. for 10 minutes and the aqueous phase was transferred to a fresh microfuge tube and an equal volume of 70% ethanol was added. The sample was loaded on an RNeasy Mini spin column, placed in a 2 mL collection tube and processed according to the manufacturer's instructions. Total RNA was extracted by elution, directly to the spin column membrane with 100 μL RNase-free water. The quality of the RNA preparations was determined by fractionating 3 μL in a 1% agarose gel before being stored at −80° C. until used.

The variable region of the Ig heavy chain of each hybridoma was amplified using a 5′ primer mix comprising 32 mouse specific leader sequence primers designed to target the complete mouse V_{H} repertoire in combination with a 3′ mouse Cγ primer specific for all mouse Ig isotypes. Similarly, a primer mix containing thirty two 5′ Vκ leader sequences designed to amplify each of the Vκ mouse families was used in combination with a single reverse primer specific to the mouse kappa constant region in order to amplify and sequence the kappa light chain. For antibodies containing a lambda light chain, amplification was performed using three 5′ V_{L} leader sequences in combination with one reverse primer specific to the mouse lambda constant region. The V_{H} and V_{L} transcripts were amplified from 100 ng total RNA using the Qiagen One Step RT-PCR kit as follows. A total of eight RT-PCR reactions were run for each hybridoma, four for the Vκ light chain and four for the Vγ heavy chain. PCR reaction mixtures included 3 μL of RNA, 0.5 μL of 100 μM of either heavy chain or kappa light chain primers (custom synthesized by Integrated Data Technologies), 5 μL of 5× RT-PCR buffer, 1 μL dNTPs, 1 μL of enzyme mix containing reverse transcriptase and DNA polymerase, and 0.4 μL of ribonuclease inhibitor RNasin (1 unit). The thermal cycler program was RT step 50° C. for 30 minutes, 95° C. for 15 minutes followed by 30 cycles of (95° C. for 30 seconds, 48° C. for 30 seconds, 72° C. for 1 minute). There was then a final incubation at 72° C. for 10 minutes.

The extracted PCR products were sequenced using the same specific variable region primers as described above for the amplification of the variable regions. To prepare the PCR products for direct DNA sequencing, they were purified using the QIAquick™ PCR Purification Kit (Qiagen) according to the manufacturer's protocol. The DNA was eluted from the spin column using 50 μL of sterile water and then sequenced directly from both strands (MCLAB).

Selected nucleotide sequences were analyzed using the IMGT sequence analysis tool (http://www.imgt.org/IMGTmedical/sequence_analysis.html) to identify germline V, D and J gene members with the highest sequence homology. These derived sequences were compared to known germline DNA sequences of the Ig V- and J-regions by alignment of V_{H} and V_{L} genes to the mouse germline database using a proprietary antibody sequence database.

The derived sequences of the murine heavy and light chain variable regions are provided in the appended sequence listing and, in an annotated form, PCT/US14/17810 which is incorporated herein by reference with respect to such sequences.

### Example 3

### Generation of Humanized Anti-DLL3 Antibodies

Certain murine antibodies generated as per Example 1 (termed SC16.13, SC16.15, SC16.25, SC16.34 and SC16.56) were used to derive humanized antibodies comprising murine CDRs grafted into a human acceptor antibody. In preferred embodiments the humanized heavy and light chain variable regions described in the instant Example may be incorporated in the disclosed site-specific conjugates as described below.

In this respect the murine antibodies were humanized with the assistance of a proprietary computer-aided CDR-grafting method (Abysis Database, UCL Business) and standard molecular engineering techniques as follows. Total RNA was extracted from the hybridomas and amplified as set forth in Example 2. Data regarding V, D and J gene segments of the V_{H} and V_{L} chains of the murine antibodies was obtained from the derived nucleic acid sequences. Human framework regions were selected and/or designed based on the highest homology between the framework sequences and CDR canonical structures of human germline antibody sequences, and the framework sequences and CDRs of the selected murine antibodies. For the purpose of the analysis the assignment of amino acids to each of the CDR domains was done in accordance with Kabat et al. numbering. Once the human receptor variable region frameworks are selected and combined with murine CDRs, the integrated heavy and light chain variable region sequences are generated synthetically (Integrated DNA Technologies) comprising appropriate restriction sites.

The humanized variable regions are then expressed as components of engineered full length heavy and light chains to provide the site-specific antibodies as described herein. More specifically, humanized anti-DLL3 engineered antibodies were generated using art-recognized techniques as follows. Primer sets specific to the leader sequence of the V_{H} and V_{L} chain of the antibody were designed using the following restriction sites: AgeI and XhoI for the V_{H} fragments, and XmaI and DraIII for the V_{L} fragments. PCR products were purified with a Qiaquick PCR purification kit (Qiagen), followed by digestion with restriction enzymes AgeI and XhoI for the V_{H} fragments and XmaI and DraIII for the V_{L} fragments. The V_{H} and V_{L} digested PCR products were purified and ligated, respectively, into a human IgG heavy chain constant region expression vector or a kappa C_{L} human light chain constant region expression vector. As discussed in detail below the heavy and/or light chain constant regions may be engineered to present site-specific conjugation sites on the assembled antibody.

The ligation reactions were performed as follows in a total volume of 10 μL with 200U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto ampicillin plates at a concentration of 100 μg/mL. Following purification and digestion of the amplified ligation products, the V_{H} fragment was cloned into the AgeI-XhoI restriction sites of the pEE6.4HulgG1 expression vector (Lonza) and the V_{L} fragment was cloned into the XmaI-DraIII restriction sites of the pEE12.4Hu-Kappa expression vector (Lonza) where either the HuIgG1 and/or Hu-Kappa expression vector may comprise either a native or an engineered constant region.

The humanized antibodies were expressed by co-transfection of HEK-293T cells with pEE6.4HulgG1 and pEE12.4Hu-Kappa expression vectors. Prior to transfection the HEK-293T cells were cultured in 150 mm plates under standard conditions in Dulbecco's Modified Eagle's Medium (DMEM) supplemented with 10% heat inactivated FCS, 100 μg/mL streptomycin and 100 U/mL penicillin G. For transient transfections cells were grown to 80% confluency. 12.5 μg each of pEE6.4HulgG1 and pEE12.4Hu-Kappa vector DNA were added to 50 μL HEK-293T transfection reagent in 1.5 mL Opti-MEM. The mix was incubated for 30 minutes at room temperature and plated. Supernatants were harvested three to six days after transfection. Culture supernatants containing recombinant humanized antibodies were cleared from cell debris by centrifugation at 800×g for 10 minutes and stored at 4° C. Recombinant humanized antibodies were purified by MabSelect SuRe Protein A affinity chromatography (GE Life Sciences). For larger scale antibody expression, CHO—S cells were transiently transfected in 1 L volumes, seeded at 2.2e6 cells per mL Polyethylenimine (PEI) was used as a transfection reagent. After 7-10 days of antibody expression, culture supernatants containing recombinant antibodies were cleared from cell debris by centrifugation and purified by MabSelect SuRe Protein A affinity chromatography.

The genetic composition for the selected human acceptor variable regions are shown in Table 5 immediately below for each of the humanized DLL3 antibodies. The sequences depicted in Table 5 correspond to the annotated heavy and light chain sequences set forth in FIGS. 2A and 2B for the subject clones. Note that the complementarity determining regions and framework regions set forth in FIGS. 2A and 2B are defined as per Kabat et al. (supra) using a proprietary version of the Abysis database (Abysis Database, UCL Business).

More specifically, the entries in Table 5 below correspond to the contiguous variable region sequences set forth SEQ ID NOS: 389 and 391 (hSC16.13), SEQ ID NOS: 393 and 395 (hSC16.15), SEQ ID NOS: 397 and 399 (hSC16.25), SEQ ID NOS: 401 and 403 (hSC16.34) and SEQ ID NOS: 405 and 407 (hSC16.56). Besides the genetic composition TABLE 5 shows that, in these selected embodiments, no framework changes or back mutations were necessary to maintain the favorable binding properties of the selected antibodies. Of course, in other CDR grafted constructs it will be appreciated that such framework changes or back mutations may be desirable and as such, are expressly contemplated as being within the scope of the instant invention.

TABLE 5

| | | human | FW | human | human | FW |
|---|---|---|---|---|---|---|
| mAb | human VH | JH | changes | VK | JK | changes |
| hSC16.13 | IGHV2- | JH6 | None | IGKV1- | JK1 | None |
| | 5*01 | | | 39*01 | | |
| hSC16.15 | IGHV1- | JH4 | None | IGKV1- | JK4 | None |
| | 46*01 | | | 13*02 | | |
| hSC16.25 | IGHV2- | JH6 | None | IGKV6- | JK2 | None |
| | 5*01 | | | 21*01 | | |
| hSC16.34 | IGHV1- | JH4 | None | IGKV1- | JK1 | None |
| | 3*02 | | | 27*01 | | |
| hSC16.56 | IGHV1- | JH4 | None | IGKV3- | JK2 | None |
| | 18*01 | | | 15*01 | | |

Though no residues were altered in the framework regions, in one of humanized clones (hSC16.13) mutations were introduced into heavy chain CDR2 to address stability concerns. The binding affinity of the antibody with the modified CDR was evaluated to ensure that it was equivalent to either the corresponding murine antibody.

Following humanization of all selected antibodies by CDR grafting, the resulting light and heavy chain variable region amino acid sequences were analyzed to determine their homology with regard to the murine donor and human acceptor light and heavy chain variable regions. The results, shown in TABLE 6 immediately below, reveal that the humanized constructs consistently exhibited a higher homology with respect to the human acceptor sequences than with the murine donor sequences. More particularly, the murine heavy and light chain variable regions show a similar overall percentage homology to a closest match of human germline genes (85%-93%) compared with the homology of the humanized antibodies and the donor hybridoma protein sequences (74%-83%).

TABLE 6

| | | Homology to Murine |
|---|---|---|
| | Homology to Human | Parent |
| mAb | (CDR acceptor) | (CDR donor) |
| hSC16.13 HC | 93% | 81% |
| hSC16.13 LC | 87% | 77% |
| hSC16.15 HC | 85% | 83% |
| hSC16.15 LC | 85% | 83% |
| hSC16.25 HC | 91% | 83% |
| hSC16.25 LC | 85% | 79% |
| hSC16.34 HC | 87% | 79% |
| hSC16.34 LC | 85% | 81% |
| hSC16.56 HC | 87% | 74% |
| hSC16.56 LC | 87% | 76% |

Upon testing each of the derived humanized constructs exhibited favorable binding characteristics roughly comparable to those shown by the murine parent antibodies.

### Example 4

### Fabrication of Site-Specific Anti-DLL3 Antibodies

Four engineered human IgG1/kappa anti-DLL3 site-specific antibodies were constructed. Two of the four engineered antibodies comprised a native light chain constant regions and had mutations in the heavy chain, wherein cysteine 220 (C220) in the upper hinge region of the heavy chain, which forms an interchain disulfide bond with cysteine 214 in the light chain, was either substituted with serine (C220S) or removed (C220Δ). The remaining two engineered antibodies comprised a native heavy chain constant regions and a mutated light chain, wherein cysteine 214 of the light chain was either substituted with serine (C214S) or removed (C214Δ). When assembled the heavy and light chains form antibodies comprising two free cysteines that are suitable for conjugation to a therapeutic agent. Amino acid sequences for the heavy and light antibody chains for each of the exemplary SC16.56 constructs are shown in FIGS. 3A and 3B while Table 7 immediately below summarizes the alterations. With regard to FIGS. 3A and 3B the reactive cysteine is underlined as is the mutated residue (in ss1 and ss4) at position 220 for the heavy chain and position 214 for the light chain. Unless otherwise noted, all numbering of constant region residues is in accordance with the EU numbering scheme as set forth in Kabat et al.

TABLE 7

| | Antibody | | Const. Reg. | SC16.56 |
|---|---|---|---|---|
| Designation | Component | Alteration | SEQ ID NO: | SEQ ID NO: |
| ss1 | Heavy Chain | C220S | 7 | 16 |
| | Light Chain | WT | 5 | 14 |
| ss2 | Heavy Chain | C220Δ | 8 | 17 |
| | Light Chain | WT | 5 | 14 |
| ss3 | Heavy Chain | WT | 6 | 15 |
| | Light Chain | C214Δ | 9 | 18 |
| ss4 | Heavy Chain | WT | 6 | 15 |
| | Light Chain | C214S | 10 | 19 |

The engineered antibodies were generated as follows.

An expression vector encoding the humanized anti-DLL3 antibody hSC16.56 light chain (SEQ ID NO: 14) or heavy chain (SEQ ID NO: 15) derived as set forth in Example 3 were used as a template for PCR amplification and site directed mutagenesis. Site directed mutagenesis was performed using the Quick-change® system (Agilent Technologies) according to the manufacturer's instructions.

For the two heavy chain mutants, the vector encoding the mutant C220S or C220Δ heavy chain of hSC16.56 was co-transfected with the native IgG kappa light chain of hSC16.56 in CHO—S cells and expressed using a mammalian transient expression system. The engineered anti-DLL3 site-specific antibodies containing the C220S or C220Δ mutants were termed hSC16.56ss1 (SEQ ID NOS: 16 and 14) or hSC16.56ss2 (SEQ ID NOS: 17 and 14) respectively.

For the two light chain mutants, the vector encoding the mutant C214S or C214Δ light chain of hSC16.56 was co-transfected with the native IgG heavy chain of hSC16.56 in CHO—S cells and expressed using a mammalian transient expression system. The engineered antibodies were purified using protein A chromatography (MabSelect SuRe) and stored in appropriate buffer. The engineered anti-DLL3 site-specific antibodies containing the C214S or C214Δ mutants were termed hSC16.56ss3 (SEQ ID NOS: 15 and 18) or hSC16.56ss4 (SEQ ID NOS: 15 and 19) respectively.

The engineered anti-DLL3 antibodies were characterized by SDS-PAGE to confirm that the correct mutants had been generated. SDS-PAGE was conducted on a pre-cast 10% Tris-Glycine mini gel from life technologies in the presence and absence of a reducing agent such as DTT (dithiothreitol). Following electrophoresis, the gels were stained with a colloidal coomassie solution.

Band patterns of the two heavy chain (HC) mutants, hSC16.56ss1 (C220S) and hSC16.56ss2 (C220Δ) and the two light chain (LC) mutants, hSC16.56ss3 (C214S) and hSC16.56ss4 (C214Δ) were observed. Under reducing conditions, for each antibody, two bands corresponding to the free LCs and free HCs, were observed. This pattern is typical of IgG molecules in reducing conditions. Under non-reducing conditions, the four engineered antibodies (hSC16.56ss1-hSC16.56ss4) exhibited band patterns that were different from native IgG molecules, indicative of the absence of a disulfide bond between the HC and LC. All four mutants exhibited a band around 98 kD corresponding to the HC-HC dimer. The mutants with a deletion or mutation on the LC (hSC16.56ss3 and hSC16.56ss4) exhibited a single band around 24 kD corresponding to a free LC. The engineered antibodies containing a deletion or mutation on the heavy chain (hSC16.56ss1 and hSC16.56ss2) had a faint band corresponding to the free LC and a predominant band around 48 kD that corresponded to a LC-LC dimer. The formation of some amount of LC-LC species is expected with the ss1 and ss2 constructs due to the free cysteines on the c-terminus of each light chain.

### Example 5

### Conjugation of Site-Specific Antibodies

A site-specific antibody (hSC16.56ss1) fabricated as set forth in Example 4 above was completely reduced using DTT or partially reduced using TCEP (tris(2-carboxyethyl)phosphine) prior to conjugation with linker-drug comprising a PBD in order to demonstrate site-specific conjugation. Unless otherwise noted PBD5 was used in all the following examples.

A schematic diagram of the process can be seen in FIG. 4. The target conjugation site for this construct is the unpaired cysteine (C214) on each light chain constant region. Conjugation efficiency (on-target and off-target conjugation) can be monitored using a reverse-phase (RP-HPLC) assay that can track on-target conjugation on the light chain vs. off-target conjugation on the heavy chain. A hydrophobic interaction chromatography (HIC) assay may be used to monitor the distribution of drug to antibody ratio species (DAR). In this example, the desired product is an ADC that is maximally conjugated on the light chain (on-target) as determined by reverse-phase chromatography and that minimizes over-conjugated (DAR>2) species while maximizing DAR=2 species.

Different preparations of hSC16.56ss1 were either completely reduced with a 40 molar equivalent addition of 10 mM DTT or partially reduced with a 2.6 molar equivalent addition of 10 mM TCEP.

Samples reduced with 10 mM DTT were reduced overnight (>12 h) at room temperature prior to buffer exchange into a Tris pH 7.5 buffer using a 30 kDa membrane (Millipore Amicon Ultra) and the equivalent of 10 diavolumes of buffer exchange. The resulting fully reduced preparations were then re-oxidized with 4.0 molar equivalent addition of 10 mM dehydroascorbic acid (DHAA) in dimethylacetamide (DMA). When the free thiol concentrations (number of free thiols per antibody, as measured by Ellman's method) of the samples were between 1.9 and 2.3, the free cysteines of the antibodies were conjugated to PBD cytotoxins via a maleimido linker for a minimum of 30 minutes at room temperature. The reaction was then quenched with the addition of 1.2 molar excess of N-acetyl-cysteine (NAC) using a 10 mM stock solution prepared in water. After a minimum quench time of 20 minutes, the pH was adjusted to 6.0 with the addition of 0.5 M acetic acid. The various conjugated preparations of antibody-PBD were then buffer exchanged into 20 mM histidine chloride pH 6.0 by diafiltration using a 30 kDa membrane.

The samples partially reduced with 10 mM TCEP were reduced for a minimum of 90 minutes at room temperature. When the free thiol concentrations of the samples were between 1.9 and 2.3, the partially reduced antibodies were conjugated to a PBD, again via a maleimido linker, for a minimum of 30 minutes at room temperature. The reaction was then quenched with the addition of 1.2 molar excess NAC from a 10 mM stock solution prepared in water. After a minimum quench time of 20 minutes, the pH was adjusted to 6.0 with the addition of 0.5 M acetic acid. The preparations of conjugated antibody-PBD were then buffer exchanged into 20 mM histidine chloride pH 6.0 by diafiltration using a 30 kDa membrane.

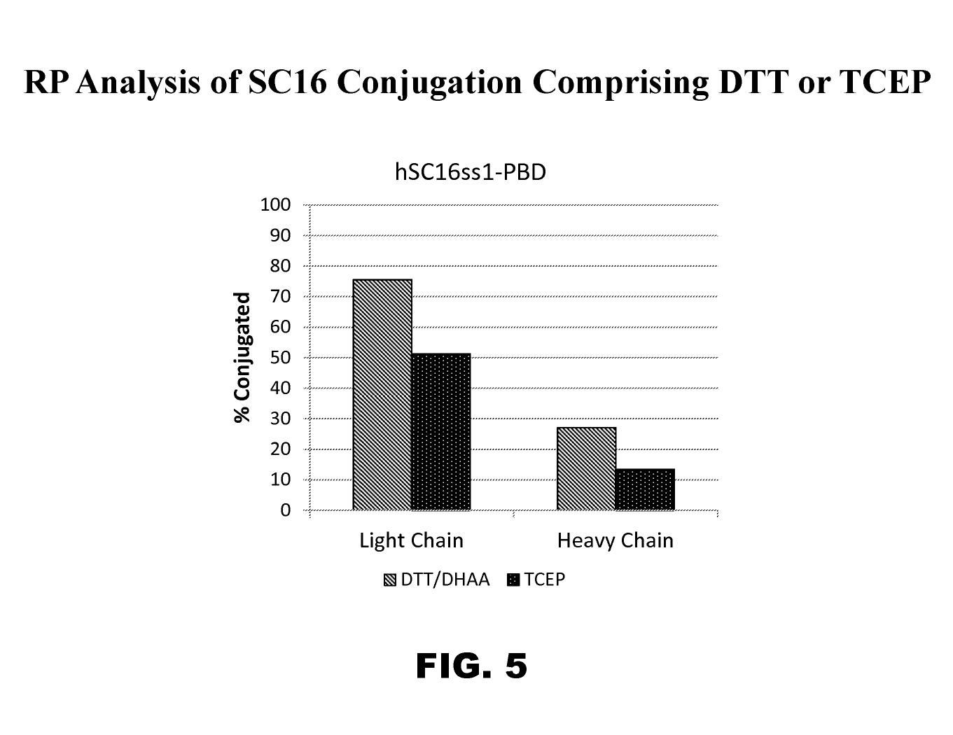

The final antibody-drug preparations (both DTT reduced and TCEP reduced) were analyzed using RP-HPLC to quantify heavy vs. light chain conjugation sites in order to determine the percentage of on-target light-chain conjugation (FIG. 5). The analysis employed an Aeris WIDEPORE 3.6 am C4 column (Phenomenex) with 0.1% v/v TFA in water as mobile phase A, and 0.1% v/v TFA in 90% v/v acetonitrile as mobile phase B. Samples were fully reduced with DTT prior to analysis, then injected onto the column, where a gradient of 30-50% mobile phase B was applied over 10 minutes. UV signal at 214 nm was collected and then used to calculate the extent of heavy and light chain conjugation.

More particularly the distribution of payloads between heavy and light chains in hSC16.56ss1-PBD conjugated using DTT and TCEP are shown in FIG. 5. Percent conjugation on the heavy and light chains were performed by integrating the area under the RP-HPLC curve of the previously established peaks (light chain, light chain+1 drug, heavy chain, heavy chain+1 drug, heavy chain+2 drugs, etc) and calculating the % conjugated for each chain separately. As discussed throughout the instant specification selected embodiments of the invention comprise conjugation procedures that favor placement of the payload on the light chain.

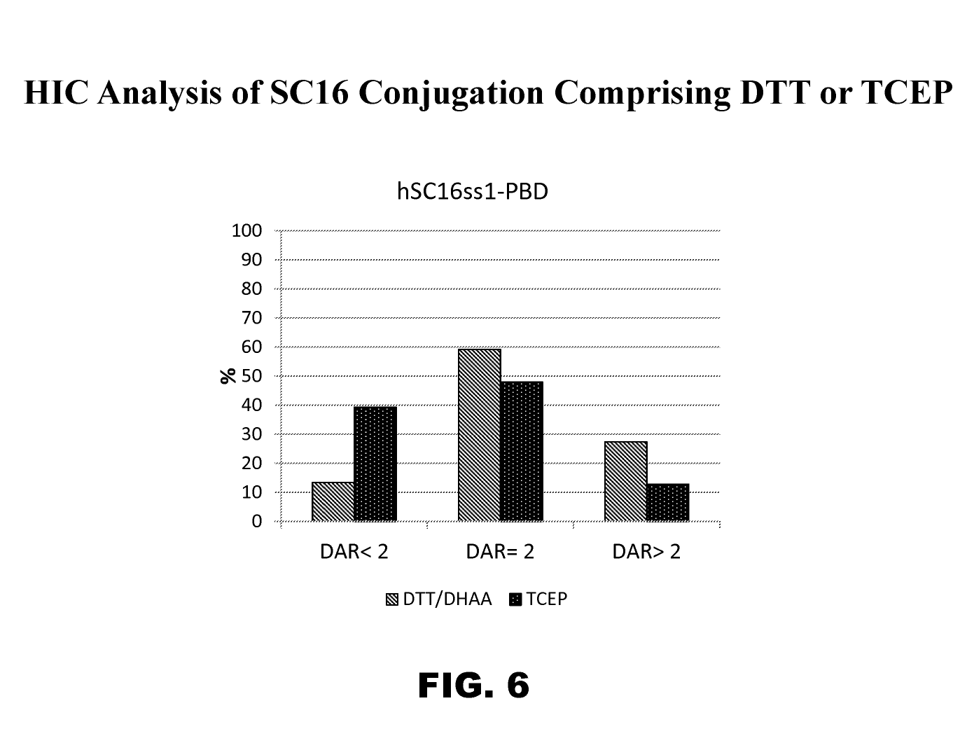

The same preparations were also analyzed using HIC to determine the amount of DAR=2 species relative to the unwanted DAR>2 species (FIG. 6). In this regard HIC was conducted using a PolyPROPYL A 3 am column (PolyLC) with 1.5M ammonium sulfate and 25 mM potassium phosphate in water as mobile phase A, and 0.25% w/v CHAPS and 25 mM potassium phosphate in water as mobile phase B. Samples were injected directly onto the column, where a gradient of 0-100% mobile phase B was applied over 15 minutes. UV signal at 280 nm was collected, and the chromatogram analyzed for unconjugated antibody and higher DAR species. DAR calculations were performed by integrating the area under the HIC curve of the previously established peaks (DAR=0, DAR=1, DAR=2, DAR=4, etc) and calculating the % of each peak. The resulting DAR distribution in hSC16.56ss1-PBD conjugated using DTT and TCEP are shown in FIG. 6.

The DAR distributions as determined by HIC of the hSC16 site-specific conjugate preparations indicate that the DTT/DHAA full reduction and reoxidation method results in ˜60% DAR=2 species, whereas the typical partial TCEP reduction method results in ˜50% DAR=2. The full reduction and reoxidation method also results in higher unwanted DAR>2 species (20-25%) while the partial TCEP reduction method results in 10-15% DAR>2 (FIG. 6). Note that while the TCEP partial reduction had lower levels of DAR>2 species, the DAR=2 percentage is only 50%. Driving up the % DAR=2 species in the TCEP system would result in a corresponding increase in the unwanted DAR>2 species. The increase in high DAR species for the DTT/DHAA full reduction samples can be attributed to higher off-target conjugation on the heavy chain as shown by RP-HPLC (FIG. 5), which is due to non-specific reduction of the hinge region cysteine residues as the driving force for reduction is increased. Thus, while the disclosed site-specific constructs provide improved DAR and less unwanted higher DAR impurities relative to native antibodies, conventional reduction methods generate at least some non-specific conjugates comprising cytotoxic agents on cysteine residues that are different from the intended engineered sites.

### Example 6

### Conjugation of Engineered Antibodies Using a Selective Reduction Process

In order to further improve the specificity of the conjugation and homogeneity of the final product site-specific antibodies fabricated as set forth in Example 4 were selectively reduced using a novel process comprising a stabilizing agent (e.g. L-arginine) and a mild reducing agent (e.g. glutathione) prior to conjugation with linker-drug comprising a PBD. As discussed above, selective conjugation preferentially conjugates the PBDs on the free cysteine with a little non-specific conjugation.

Per Example 4, the target conjugation site for the hSC16.56ss1 construct is the unpaired cysteine on each light chain. In order to direct conjugation to these engineered sites, preparations of hSC16.56ss1 were partially reduced in a buffer containing 1M L-arginine/5 mM glutathione, reduced (GSH)/5 mM EDTA, pH 8.0 for a minimum of one hour at room temperature. Additionally, as controls, each antibody preparation was separately incubated in 1M L-arginine/5 mM EDTA, pH 8.0 and 20 mM Tris/3.2 mM EDTA/5 mM GSH, pH 8.2 buffers for one hour or longer. All preparations were then buffer exchanged into a 20 mM Tris/3.2 mM EDTA, pH 8.2 buffer using a 30 kDa membrane (Millipore Amicon Ultra). The resulting partially reduced preparations (for samples incubated in arginine and glutathione together) had free thiol concentrations between 1.9 and 2.3, and all preparations were then conjugated to a PBD via a maleimido linker for a minimum of 30 minutes at room temperature. The reaction was then quenched with the addition of 1.2 molar excess of NAC using a 10 mM stock solution prepared in water. After a minimum quench time of 20 minutes, the pH was adjusted to 6.0 with the addition of 0.5 M acetic acid. The various conjugated preparations of antibody-PBD were then diafiltered into 20 mM histidine chloride, pH 6.0 by diafiltration using a 30 kDa membrane.

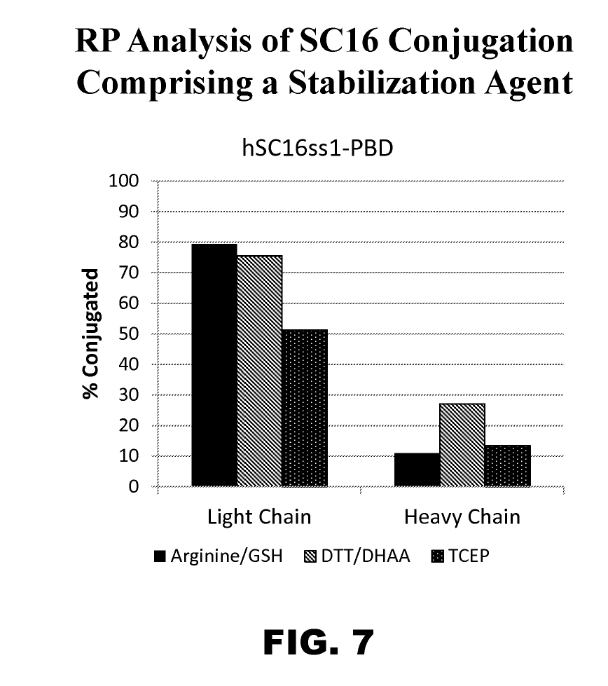

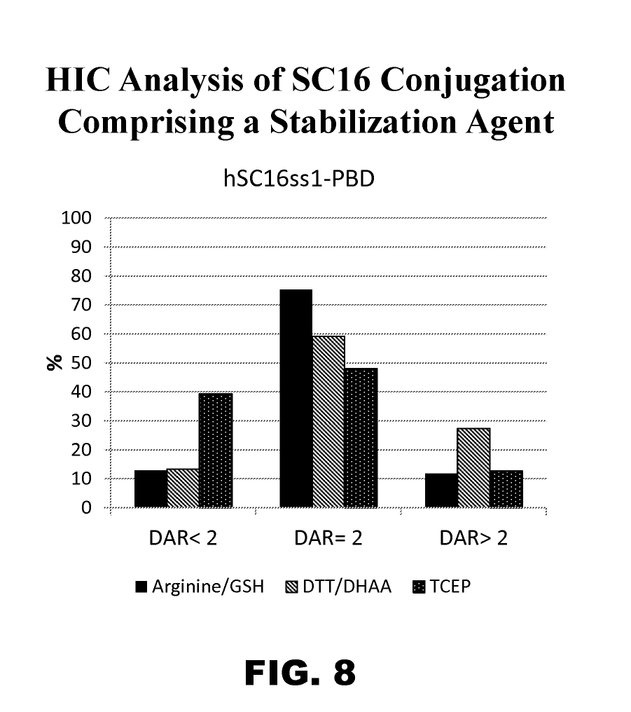

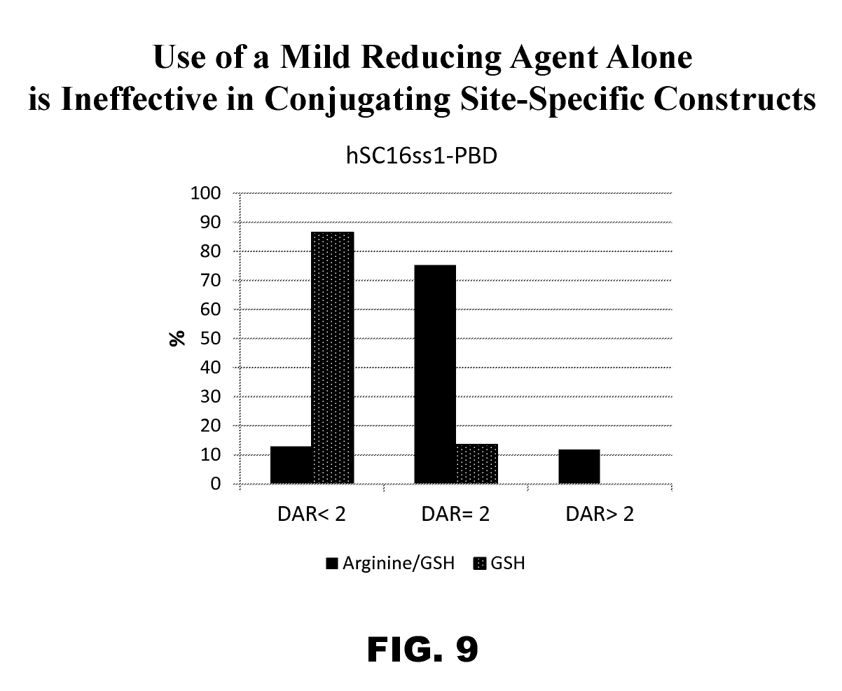

The final antibody-drug preparations were analyzed using RP-HPLC as previously discussed to quantify heavy vs. light chain conjugation sites in order to determine the percentage of on-target light-chain conjugation (FIG. 7). The samples were also analyzed using hydrophobic interaction chromatography to determine the amount of DAR=2 species relative to the unwanted DAR>2 species (FIG. 8). For comparative purposes results obtained in the previous Example are included in FIGS. 7 and 8 for DTT/DHAA and TCEP reduced samples. HIC analysis of the EDTA/GSH controls are presented in FIG. 9 where they are shown next to the selectively reduced samples.

FIGS. 7 and 8 summarize the HIC DAR distributions and the % conjugated light chain of the antibodies reduced using the selective reduction process compared to standard complete or partial reduction processes (as described in Example 6). The benefit of the selective conjugation method in combination with the engineered constructs is readily apparent, resulting in superior selectivity of the desired light chain conjugation site (FIG. 7) and providing an average DAR=2 level of 60-75% while maintaining unwanted DAR>2 species below 15% (FIG. 8). The results shown in FIGS. 7 and 8 demonstrate that selective reduction drives the reaction to provide higher levels of DAR=2 and less of the undesired DAR>2 species than the standard partial or complete reduction procedures. Control procedures shown in FIG. 9 demonstrate that the mild reducing agent (e.g. GSH) cannot effect the desired conjugation in the absence of a stabilizing agent (e.g. L-arginine). Control procedures shown in FIG. 9 demonstrate that the mild reducing agent (e.g. GSH) cannot effect the desired conjugation in the absence of a stabilizing agent (e.g. L-arginine).

These data demonstrate that selective reduction provides advantages over conventional partial and complete reduction conjugation methods. This is particularly true when the novel selective reduction procedures are used in conjunction with antibodies engineered to provide unpaired (or free) cysteine residues. Mild reduction in combination with a stabilizing agent (i.e., selective reduction) produced stable free thiols that were readily conjugated to various linker-drugs, whereas DHAA reoxidation is time sensitive and TCEP reduction was not as successful, particularly for the engineered constructs described here.

### Example 7

### Selective Reduction with Different Systems

To further demonstrate the advantages of selective reduction using various combinations of stabilizing agents and reducing agents, hSC16.56ss1 was selectively reduced using different stabilizing agents (e.g. L-lysine) in combination with different mild reducing agents (e.g. N-acetyl-cysteine or NAC) prior to conjugation.

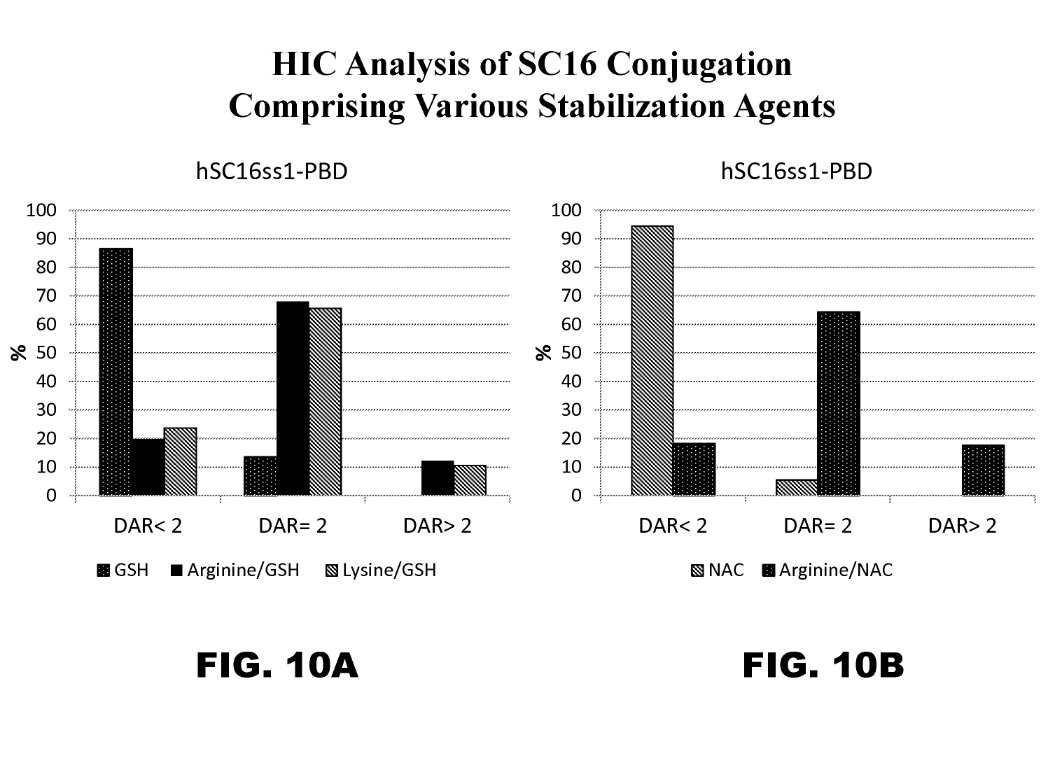

Three preparations of hSC16.56ss1 were selectively reduced using three different buffer systems: (1) 1M L-arginine/6 mM GSH/5 mM EDTA, pH 8.0, (2) 1M L-arginine/10 mM NAC/5 mM EDTA, pH 8.0, and (3) 1M L-Lysine/5 mM GSH/5 mM EDTA, pH 8.0. Additionally, as controls, the antibody preparations were separately incubated in 20 mM Tris/5 mM EDTA/10 mM NAC, pH 8.0 and 20 mM Tris/3.2 mM EDTA/5 mM GSH, pH 8.2 buffers. All preparations were incubated for a minimum of one hour at room temperature, and then buffer exchanged into a 20 mM Tris/3.2 mM EDTA, pH 8.2 buffer by diafiltration using a 30 kDa membrane (Millipore Amicon Ultra). The resulting selectively reduced preparations, which were found to have free thiol concentrations between 1.7 and 2.4, were then conjugated to a PBD via a maleimido linker. After allowing the conjugation reaction to proceed for a minimum of 30 minutes at room temperature, the reaction was quenched with the addition of 1.2 molar excess of NAC using a 10 mM stock solution. Following a minimum quench time of 20 minutes, the pH was adjusted to 6.0 with the addition of 0.5 M acetic acid. The various conjugated preparations of antibody-PBD were then buffer exchanged into 20 mM histidine chloride pH 6.0 by diafiltration using a 30 kDa membrane. Final antibody-drug preparations were then analyzed using hydrophobic interaction chromatography to determine DAR distribution (see FIGS. 10A and 10B).

DAR distributions as determined by HIC show similar results for the three different selective reduction systems employed (Arg/GSH, Lys/GSH and Arg/NAC). More particularly, DAR=2 levels are 60-65% for the different preparations, and high-DAR species (DAR>2) are maintained below 20% for all selective reduction systems and linker-drug combinations, indicating high selectivity for the engineered cysteine residues in the constant region of the light chain. Again, as previously shown in Example 6, mild reducing agents alone (e.g. GSH or NAC) did not provide sufficient conjugation selectivity while the addition of the stabilizing agent results in significant improvement.

### Example 8

### Production of Highly Homogenous Antibody-Drug Conjugate Preparations

In order to further increase DAR homogeneity of the site-specific antibody-drug conjugates and demonstrate that such homogeneous preparations have improved therapeutic index and toxicity profiles, preparative hydrophobic interaction chromatography was used to separate the different DAR species generated by the disclosed conjugation procedures.

A preparation of hSC16.56ss1 was selectively reduced in a buffer containing 1M L-arginine/5 mM glutathione, reduced (GSH)/5 mM EDTA, pH 8.0 for a minimum of one hour at room temperature. The preparation was then buffer exchanged into a 20 mM Tris/3.2 mM EDTA, pH 8.2 buffer using a 30 kd membrane (Millipore Amicon Ultra). The resulting preparation, which had a measured free thiol concentration of 2.4, was then conjugated to a PBD via a maleimido linker. Conjugation was allowed to proceed for a minimum of 30 minutes at room temperature before the reaction was quenched with the addition of 1.2 molar excess of NAC using a 10 mM stock solution. After quenching the reaction for at least 20 minutes, the pH was adjusted to 6.0 with the addition of 0.5 M acetic acid. The conjugated antibody preparation was then diluted with a high salt buffer to increase the conductivity of the load to 100±20 mS/cm, and then loaded on a Butyl HP resin chromatography column (GE Life Sciences). A decreasing salt gradient using buffer A (25 mM potassium phosphate, 1 M ammonium sulfate, pH 6) and Buffer B (25 mM potassium phosphate, pH 6) was then employed to separate the different DAR species based on hydrophobicity, where DAR=0 species eluted first, followed by DAR=1, DAR=2, and then higher DAR species.

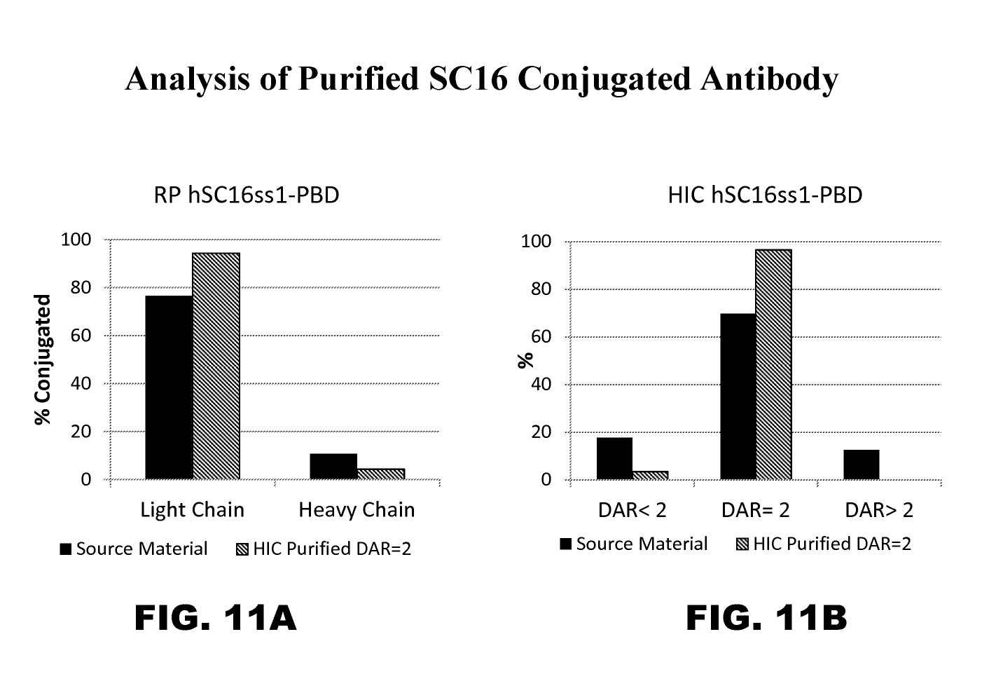

The final antibody-drug “HIC purified DAR=2” preparation was analyzed using RP-HPLC to quantify heavy vs. light chain conjugation sites in order to determine the percentage of on-target light-chain conjugation compared to the source material (FIG. 11A). The sample was also analyzed using analytical hydrophobic interaction chromatography to determine the amount of DAR=2 species relative to the unwanted DAR>2 species, and the distribution was also compared to the source material (FIG. 11B).

The HIC purification process results in DAR=2 levels greater than 95%, as well as light chain conjugation levels greater than 90%, indicating a high degree of homogeneity in the final sample with conjugation substantially limited to the desired free cysteine residues on the C-terminus of the light chain constant region. This purification process was executed successfully at several scales (data not shown), achieving reproducible high DAR=2 levels and high light chain conjugation levels from the milligram to gram scales. The process was successfully implemented to generate material for in vivo toxicology studies as described in the Examples below. It will be appreciated that this process can be further scaled and can be implemented in a GMP process to produce therapeutic material.

### Example 9

### Site-Specific Constructs Retain Binding Characteristics

Site-specific anti-DLL3 antibodies and ADCs fabricated as set forth in the previous Examples were screened by an ELISA assay to determine whether they bound to DLL3 purified protein. The parental non-engineered antibody was used, in conjugated and non-conjugated forms, as a control and run alongside the site-specific anti-DLL3 antibody and anti-DLL3 antibody drug conjugate. Binding of the antibodies to DLL3 was detected with a monoclonal antibody (mAb) reporter antibody conjugated to horseradish peroxidase (HRP), (Southern Biotech, Cat. No. SB9052-05), which binds to an epitope present on human IgG1 molecules. Binding of the ADCs (site-specific or conventional) to DLL3 was detected with R3.56 antibody conjugated to horseradish peroxidase (HRP) which binds to the drug linker on the ADC. HRP reacts with its substrate tetramethyl benzidine (TMB). The amount of hydrolyzed TMB is directly proportional to the amount of test article bound to DLL3.

ELISA plates were coated with 1 μg/ml purified DLL3 in PBS and incubated overnight at 4° C. Excess protein was removed by washing and the wells were blocked with 2% (w/v) BSA in PBS with 0.05% tween 20 (PBST), 200 μL/well for 1 hour at room temperature. After washing, 100 μL/well serially diluted antibody or ADC were added in PBST for 1 hour at room temperature. The plates were washed again and 0.5 ug/ml of 100 μL/well of the appropriate reporter antibody was added in PBST for 1 hour at room temperature. After another washing, plates were developed by the addition of 100 μL/well of the TMB substrate solution (Thermo Scientific) for 15 minutes at room temperature. An equal volume of 2 M H₂SO₄ was added to stop substrate development. The samples were then analyzed by spectrophotometer at OD 450.

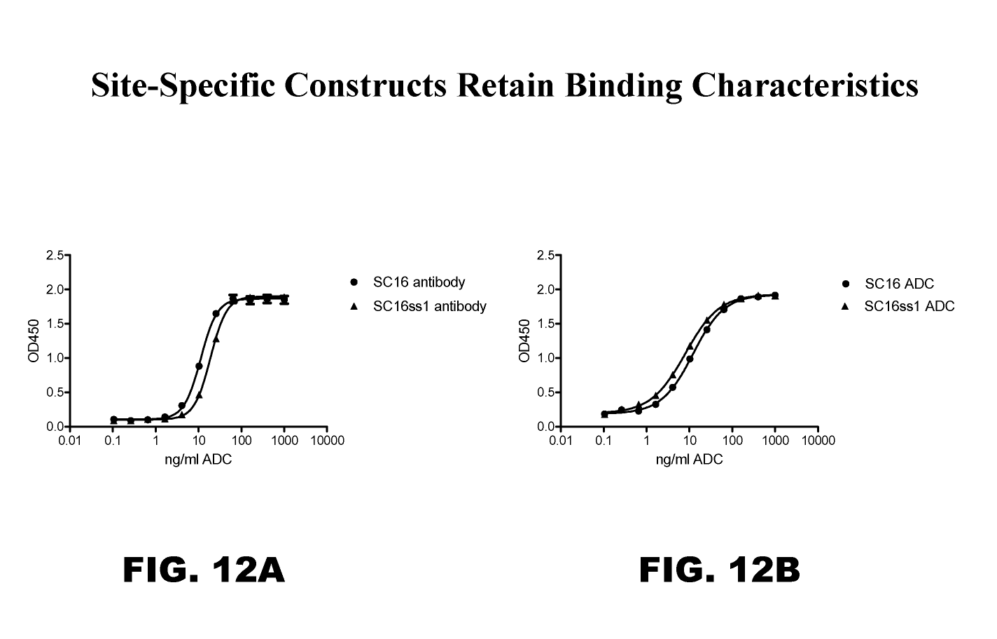

The results of the ELISAs are shown in FIGS. 12A (antibody) and 12B (ADC). A review of the data demonstrates that engineering of the heavy chain CH1 domain to provide a free cysteine on the light chain constant region did not adversely impact the binding of the antibodies to the target antigen. Similar assays (data not shown) conducted with various site-specific constructs shows that engineering of the light chain constant region or the CH1 region to provide free cysteines has little impact on the binding characteristics of the resulting antibody or ADC.

### Example 10

### In Vitro Cytotoxicity of Site-Specific Conjugates

Assays were run to demonstrate the ability of site-specific conjugates to effectively kill cells expressing the human DLL3 antigen in vitro. In this regard the assay measures the ability of anti-DLL3 site-specific conjugate to kill HEK293T cells engineered to express human DLL3. In this assay killing requires binding of the ADC (site-specific or control) to its DLL3 target on the cell surface followed by internalization of ADC. Upon internalization the linker (a Val-Ala protease cleavable linker as described above) is cleaved and releases the PBD toxin inside the cells leading to cell death. Cell death is measured using CellTiter-Glo reagent that measures ATP content as a surrogate for cell viability.

Specifically, 500 cells per well in DMEM supplemented with 10% fetal bovine serum and penicillin/streptomycin (DMEM complete media), were plated into 96 well tissue culture treated plates one day before the addition of antibody drug conjugates. 24 hours post plating cells were treated with serially diluted SC16.56-PBD control or SC16.56 ss1-PBD in DMEM complete media. The cells were cultured for 96 hours post treatment, after which, viable cell numbers were enumerated using Cell Titer Glo® (Promega) as per manufacturer's instructions.

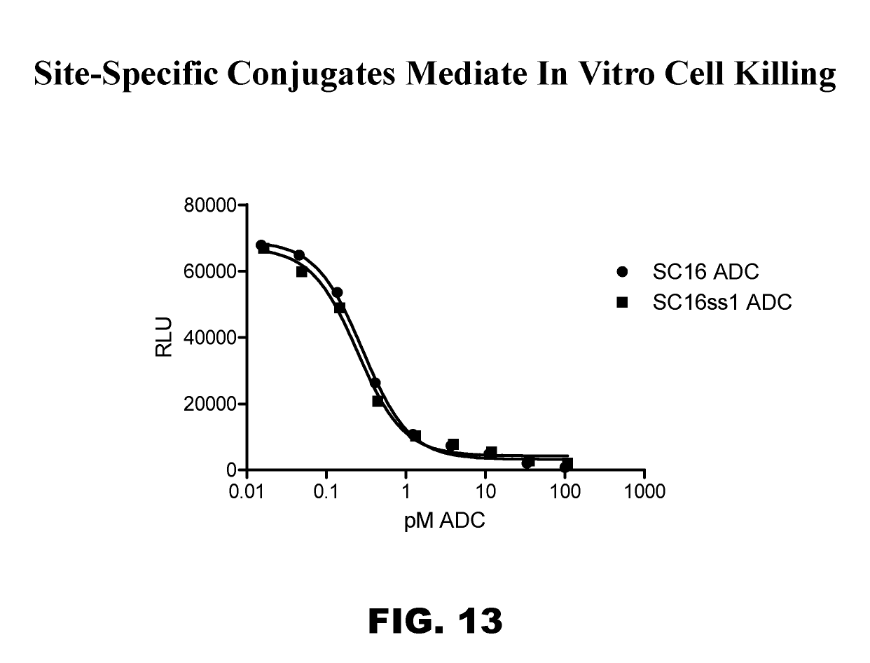

As illustrated in FIG. 13, SC16.56-PBD and SC16.56ss1-PBD both proved effective in killing cells at concentrations under 1.0 pM of ADC. These data indicate that both conventional anti-DLL3 PBD conjugates and anti-DLL3 site-specific PBD conjugates are lethal at therapeutic levels.

### Example 11

### Stability of Site-Specific Conjugates in Serum

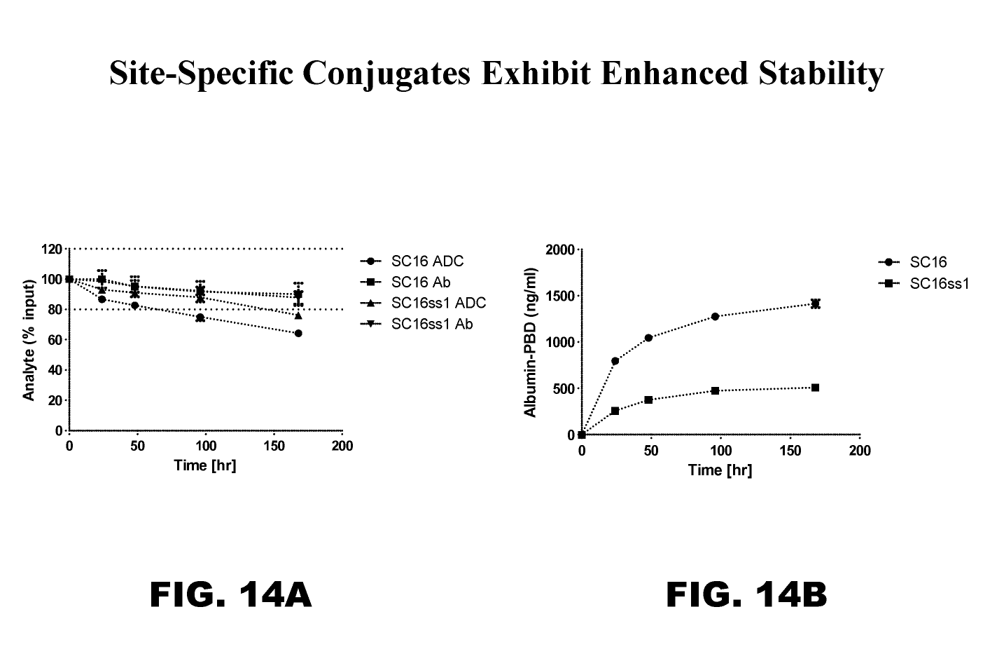

In order to demonstrate improved stability provided by the site-specific conjugates of the instant invention, selected conjugates were exposed to human serum in vitro for extended periods. Degradation of the ADCs were measured over time to provide the data set forth in FIG. 14A.

More specifically SC16 ADC and SC16ss1 ADC, each comprising the same PBD cytotoxin, were added to human serum obtained commercially (Bioreclamation) and incubated at 37° C., 5% CO2 for extended periods. Samples were collected at 0, 24, 48, 96 and 168 hours post addition and stability was measured using a sandwich ELISA to measure both total antibody content and ADC levels.

With regard to the measurement of total antibody content the ELISA is configured to detect both conjugated and unconjugated SC16 or SC16ss1 antibodies. This assay employs a pair of anti-idiotypic antibodies which specifically capture and detect SC16 and SC16ss1 with or without conjugated cytotoxins. Mechanically the assay is run using the MSD Technology Platform (Meso Scale Diagnostics, LLC) which uses electrochemiluminescence for increased sensitivity and linearity.

To this end MSD high bind plates were coated overnight at 4° C. with 2 ug/mL capture anti-idiotypic (ID-16) antibody. Next day, plates were washed with PBST (PBS+0.05% Tween20) and blocked with 150 uL 3% BSA in PBST. 25 uL serum samples, along with ADC standard curve were added to the plate and allowed to incubate for 2 hours at room temperature. After incubation, plates were washed with PBST and 25 uL sulfo-tagged detection anti-idiotypic (ID-36) antibody at 0.5 ug/mL was added to each well and incubated for 1 hour at room temperature. Plates were then washed and 150 uL 1×MSD read buffer was added per well and read out with the MSD reader.

Data in FIG. 14A is graphed as percent of total ADC initially added into the human serum. FIG. 14A shows that antibody levels of SC16 and SC16ss1 (SC16 Ab and SC16ss1 Ab in the legend) essentially remain stable over the course of 168 hours at 37° C. Further monitoring showed there was little change in total antibody concentration out to 336 hours (data not shown).

In addition to monitoring the total antibody concentration ELISA assays were run on the collected samples to determine levels of antibody drug conjugate remaining. That is, the assay measures the levels of intact SC16-PBD and SC16ss1-PBD using the ELISA methodology generally as described immediately above. However, unlike the previous ELISA assay this ELISA quantifies the SC16 or SC16ss1 antibody conjugated to one or more PBD molecules, but cannot determine the number of PBD molecules on actually present on the detected ADC. Unlike the total antibody assay this assay uses a combination of an anti-idiotypic mAb and an anti-PBD specific mAb and does not detect the unconjugated SC16 antibody.

Again, this ELISA assay uses the MSD Technology Platform to generate the data. MSD standard bind plates were coated overnight at 4° C. with 4 ug/mL anti-PBD specific mAb (R3.56). Next day, plates were washed with PBST (PBS+0.05% Tween20) and blocked with 150 uL 3% BSA in PBST. 25 uL serum samples, along with ADC standard curve and QC samples were added to the plate and allowed to incubate for 2 hours at room temperature. After incubation, plates were washed with PBST and 25 uL sulfo-tagged detection anti-idiotypic antibody (ID-36) at 0.5 ug/mL was added to each well and incubated for 1 hour at room temperature. Plates were then washed and 150 uL 1×MSD read buffer was added per well and read out with the MSD reader. Data for samples out to 168 hours is shown in FIG. 14A (SC16 ADC and SC16ss1 ADC in the legend)

Data in FIG. 14A show that, unlike total antibody levels, the concentration of intact conventional ADCs (SC16 ADC) falls off markedly more than the concentration of intact site-specific ADC (SC16ss1 ADC). Such results indicate that ADCs conjugated at random cysteine sites tend to degrade more rapidly than the presently disclosed site-specific conjugates. As previously discussed, degradation of the ADC may lead to increased non-specific toxicity resulting from the free cytotoxin with a corresponding reduction in the therapeutic index.

### Example 12

### Albumin Transfer of Site-Specific Conjugates in Serum

With conventional ADCs it has been noted that albumin in serum can leach the conjugated cytotoxin thereby increasing non-specific cytotoxicity. In order to determine the amount of site-specific ADC degradation mediated by albumin transfer, an ELISA assay was developed to measure the amount of albumin-PBD (hAlb-PBD) in serum exposed to SC16-PBD and SC16ss1-PBD. This ELISA uses an anti-PBD specific mAb to capture hAlb-PBD and an anti-human albumin mAb is used as detection antibody. As free ADC will compete with the hAlb-PBD, serum samples must be depleted of the PBD ADC prior to testing. Quantitation is extrapolated from a hAlb-PBD standard curve. Along with the previous Example this assay uses the MSD Technology Platform to generate the data which is shown in FIG. 14B.

Initially the serum samples were inoculated with SC16-PBD or SC16ss1-PBD to a final concentration of 10 μg along with the relevant controls. As with the previous Example samples were taken at 0, 24, 48, 96 and 168 hours post addition.

As to the assay, MSD standard bind plates were coated overnight at 4° C. with 4 ug/mL anti-PBD specific mAb (R3.56). Next day, plates were washed with PBST (PBS+0.05% Tween20) and blocked with 25 uL MSD Diluent 2+0.05% Tween-20 for 30 minutes at room temperature. Serum samples were diluted 1:10 in MSD Diluent 2+0.1% Tween-20 (10 uL serum+90 uL diluent) and incubated with 20 uL GE's MabSelect SuRe Protein A resin for 1 hour on vortex shaker. After depletion of intact SC16-PBD or SC16ss1-PBD by anti-idiotypic antibodies, samples were separated from resin using 96-well 3M filter plate. 25 uL of depleted serum samples were then added to the blocked plate along with an hAlb-6.5 standard curve and incubated for 1 hour at room temperature. After incubation, the plates were washed with PBST and 25 uL of 1 ug/mL sulfo-tagged anti-human albumin mAb (Abcam ab10241) diluted in MSD Diluent 3+0.05% Tween-20 were added. The plates were then incubated for 1 hour, washed with PBST and read out with 150 uL 1×MSD read buffer.

FIG. 14B shows that substantially less hAlb-PBD was detected in all SC16ss1 ADC samples collected than in SC16 spiked samples indicating that the albumin transfer rate was slower for the site-specific conjugates. As with the previous Example, this data implies that the site-specific conjugates of the instant invention may be more stable than conventional conjugates in a physiological environment and thus exhibit an improved therapeutic index due, at least in part, to the reduction of non-specific toxicity caused by non-targeted cytotoxin (e.g., hAlb-PBD).

### Example 13

### Site-Specific Constructs Demonstrate In Vivo Efficacy

In vivo experiments were conducted to confirm the cell killing ability of the site-specific constructs demonstrated in Example 10. To this end site-specific DLL3 ADCs prepared as set forth in the previous Examples were tested for in vivo therapeutic effects in immunocompromised NODSCID mice bearing subcutaneous patient-derived xenograft (PDX) small cell lung cancer (SCLC) tumors. More particularly, anti-DLL3-PBD conjugates (SC16-ADC), HIC purified anti-DLL3-PBD conjugates (SC16-ADCD2), and HIC purified site-specific anti-DLL3-PBD conjugates (SC16ss1-ADCD2) were each tested in three different SCLC models.

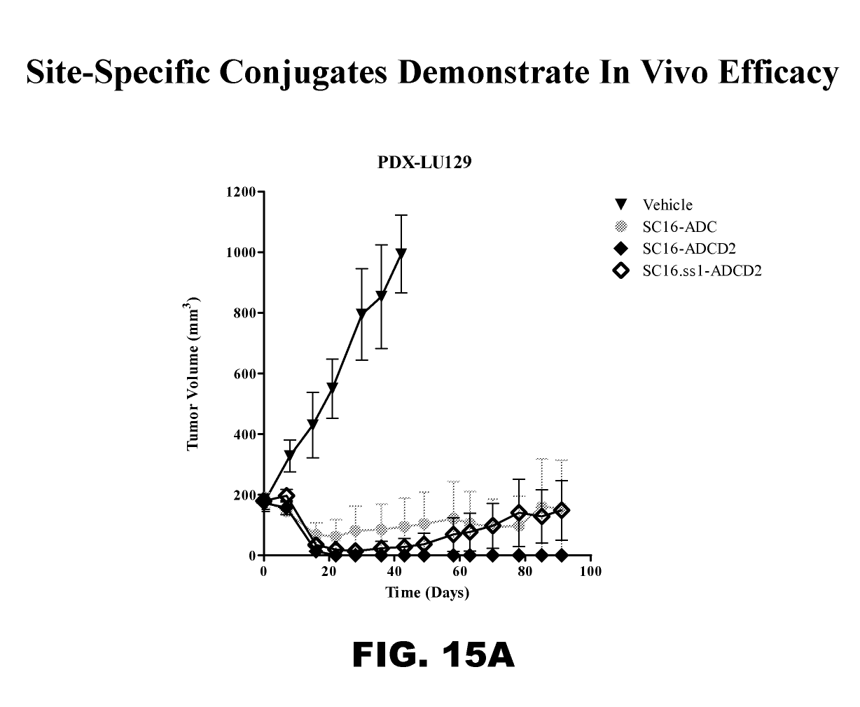

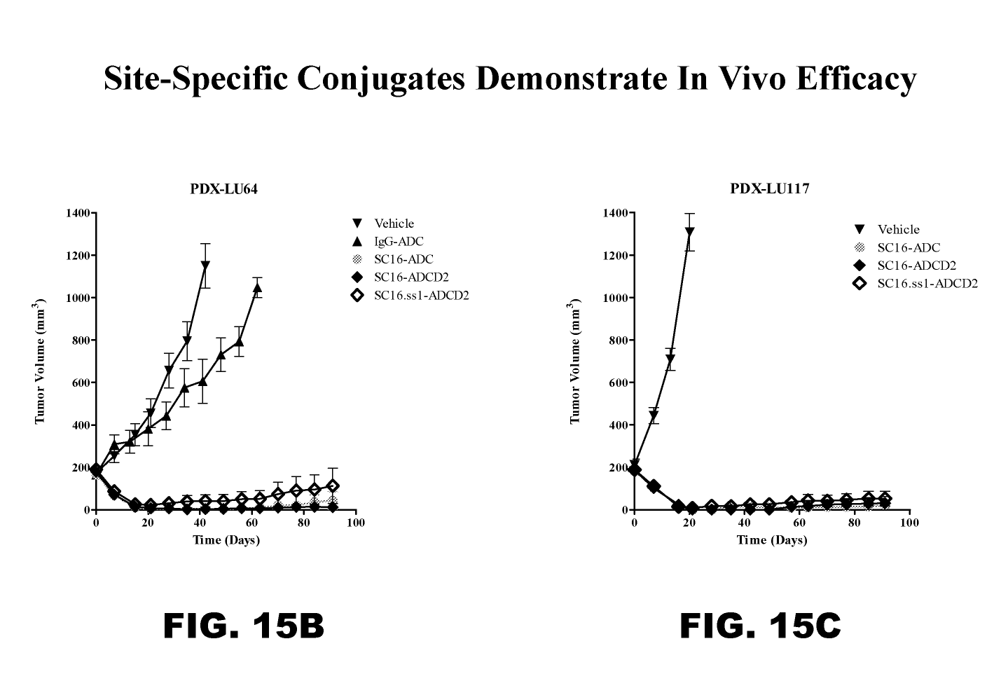

SCLC-PDX lines, LU129, LU64, and LU117 were each injected as a dissociated cell inoculum under the skin near the mammary fat pad region, and measured weekly with calipers (ellipsoid volume=a×b²/2, where a is the long diameter, and b is the short diameter of an ellipse). After tumors grew to an average size of 200 mm³ (range, 100-300 mm³), the mice were randomized into treatment groups (n=5 mice per group) of equal tumor volume averages. Mice were treated with a single dose (100 μL) with either vehicle (5% glucose in sterile water), control human IgG1 ADC (IgG-ADC; 1 mg/kg), or SC16-ADC preparations (0.75-1.5 mg/kg) via an intraperitoneal injection, with therapeutic effects assessed by weekly tumor volume (with calipers as above) and weight measurements. Endpoint criteria for individual mice or treatment groups included health assessment (any sign of sickness), weight loss (more than 20% weight loss from study start), and tumor burden (tumor volumes>1000 mm³). Efficacy was monitored by weekly tumor volume measurements (mm³) until groups reached an average of approximately 800-1000 mm³. Tumor volumes were calculated as an average with standard error mean for all mice in treatment group and were plotted versus time (days) since initial treatment. The results of the treatments are depicted in FIGS. 15A-15C where mean tumor volumes with standard error mean (SEM) in 5 mice per treatment group are shown.

DLL3-binding ADCs conjugated using either conventional (SC16-ADC or SC16-ADCD2) or site-specific strategies (SC16ss1-ADCD2) with HIC purification (in two preparations) of molecular species containing 2 drug molecules per antibody were evaluated in mice bearing SCLC PDX-LU129 (FIG. 15A; 1.5 mg/kg), PDX-LU64 (FIG. 15B; 0.75 mg/kg), or PDX-LU117 (FIG. 15C; 0.75 mg/kg) demonstrated that HIC purification and/or site-specific conjugation of DLL3-binding ADCs had similar therapeutic effects to that of conventionally conjugated SC16-ADC. Furthermore, appropriate dose levels such as those used in the present Example can achieve curative responses in SCLC PDX-bearing mice.

In these models and at the doses given site-specific and conventional ADC preparations had comparable in vivo efficacy when tested in 3 mouse models of SCLC PDX. In vivo efficacy of DLL3-binding ADCs in mice bearing SCLC-PDX tumors was also similar when comparing therapeutic effects of unpurified ADCs with DAR2-purified ADCs. Taken together, site-specific conjugation strategies and DAR2 purification methods offer comparable in vivo therapeutic efficacy to conventional, unpurified ADC conjugates.

### Example 14

### Site-Specific Conjugates Demonstrate Reduced Toxicity

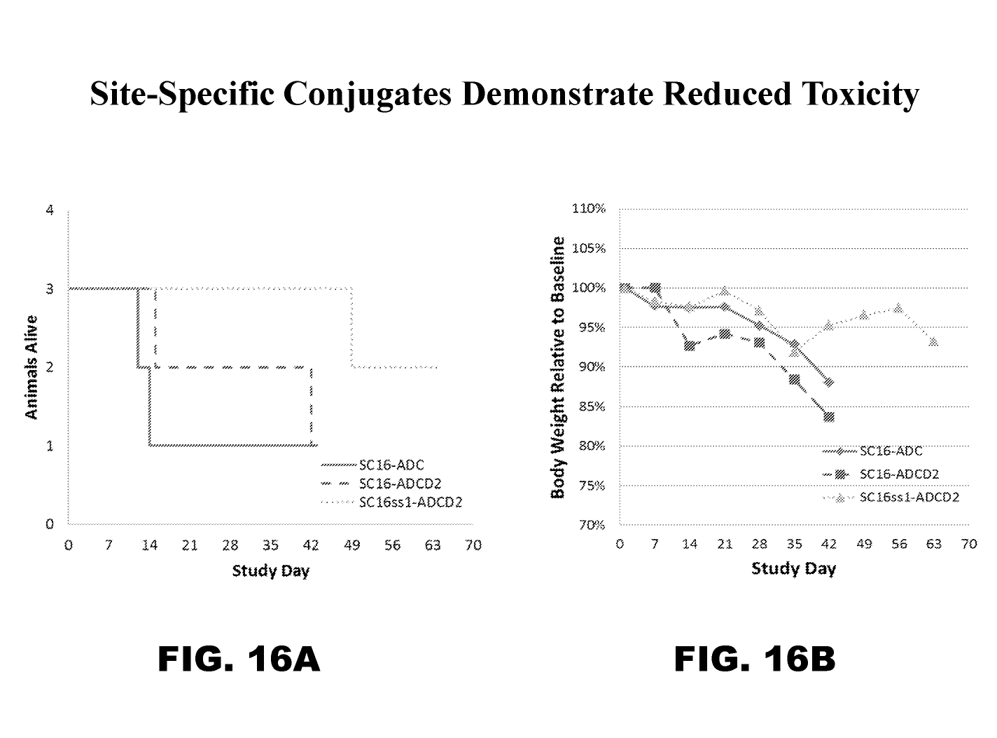

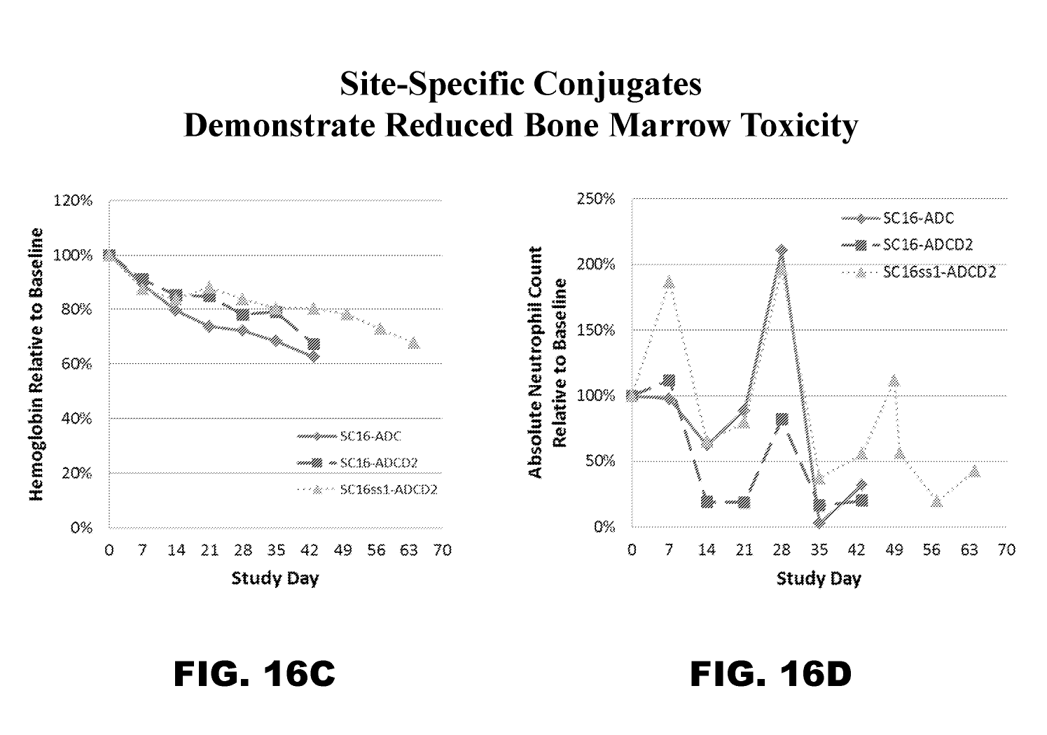

Based on the stability and efficacy data generated in the previous Examples the site-specific conjugates of the instant invention appear to exhibit a favorable clinical profile. In order to further expand the therapeutic index of the disclosed conjugate preparations studies were run to document their toxicity profile. As discussed in more detail immediately below and set forth in FIGS. 16A to 16D, the studies strongly suggested that the anti-DLL3 site-specific conjugates were better tolerated (e.g., no mortality for the same number of doses, reduced incidence of skin toxicity, reduced bone marrow toxicity, reduced severity of lymphoid tissue findings, etc.) than either native antibody anti-DLL3 conjugates or HIC purified preparations of the same. Significantly, this reduction in toxicity substantially increases the therapeutic index in that it provides for markedly higher dosing and corresponding higher localized concentrations of the cytotoxin (e.g., a PBD) at the tumor site. Based on the expected therapeutic index for the disclosed site-specific conjugates it may be possible to increase the dose (as compared to conventional native antibody conjugates) while lowering or retaining a similar level of toxicity.

With regard to the studies the toxicity of DAR2 purified site-specific ADC (SC16ss1-ADCD2) was compared to that of conventional conjugates (SC16-ADC) or DAR2 purified versions of the same (SC16-ADCD2). Each of the preparations comprise as the cytotoxin. The studies were conducted using cynomolgus monkeys as a test system. In this study, clinical signs, body weights, food consumption, clinical pathology (hematology, coagulation, clinical chemistry, and urinalysis), toxicokinetics, gross necropsy findings, organ weights, and histopathologic examinations were documented and compared.

Survival curves are shown in FIG. 16A for each of the groups dosed with SC16ss1-ADCD2, SC16-ADC and SC16-ADCD2 respectively. A review of FIG. 16A shows that survival was extended for the site-specific ADC for the same dose level and number of doses (ADCs were dosed every three weeks at the 1.25 mg/kg dose level). For SC16-ADC, two of three monkeys did not tolerate a single-dose as evidenced by moribund euthanasia. A single monkey completed two doses of the conventional ADC. For SC16-ADCD2, one of three monkeys did not tolerate a single-dose as evidence by moribund euthanasia. Of the remaining two monkeys, one did not tolerate two doses as evidence by moribund euthanasia. A single monkey completed two doses of the DAR2 purified version of the conventional ADC. Conversely, for SC16ss1-ADCD2, all three monkeys tolerated two doses. Following a third dose of the site-specific ADC, one of three monkeys was euthanized moribund. The remaining two monkeys completed three doses of the site-specific ADC and completed the study.

In addition to the survival rates shown in FIG. 16A there were reduced skin findings, better body weight maintenance (FIG. 16B), reduced bone marrow toxicity (FIGS. 16C and 16D for hemoglobin and neutrophil counts respectively), and reduced severity of lymphoid tissue findings for the site-specific ADC compared to the conventional ADC or DAR2 purified version of the conventional ADC. Taken together the results shown in FIGS. 16A-16D indicate that the site-specific conjugates of the instant invention exhibit lower toxicity than conventionally conjugated ADCs and may provide a correspondingly better therapeutic index.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. An antibody drug conjugate of the formula:
Ab-[L-D]n or a pharmaceutically acceptable salt thereof wherein
a) Ab comprises a DLL3 antibody comprising one or more unpaired cysteines;
b) L comprises an optional linker;
c) D comprises a PBD; and
d) n is an integer from about 1 to about 8.

2. The antibody drug conjugate of claim 1 wherein the DLL3 antibody comprises a monoclonal antibody.

3. The antibody drug conjugate of claims 1 or 2 wherein the DLL3 antibody comprises an internalizing antibody.

4. The antibody drug conjugate of any of claims 1 to 3 wherein the DLL3 antibody comprises a humanized antibody or a CDR grafted antibody.

5. The antibody drug conjugate of any of claims 1 to 4 wherein the DLL3 antibody comprises two unpaired cysteines.

6. The antibody drug conjugate of any of claims 1 to 5 wherein the DLL3 antibody comprises a kappa light chain.

7. The antibody drug conjugate of claim 6 wherein the DLL3 antibody comprises a light chain wherein C214 comprises an unpaired cysteine.

8. The antibody drug conjugate of any of claims 1 to 7 wherein DLL3 antibody comprises an IgG1 heavy chain.

9. The antibody drug conjugate of claim 8 wherein the DLL3 antibody comprises a heavy chain wherein C220 comprises an unpaired cysteine.

10. The antibody drug conjugate of any of claims 1 to 9 wherein the DLL3 antibody is selected from the group consisting of hSC16.13, hSC16.15, hSC16.25, hSC16.34 and hSC16.56, or an antibody that competes for binding to human DLL3 with any one of hSC16.13, hSC16.15, hSC16.25, hSC16.34 and hSC16.56.

11. The antibody drug conjugate of any of claims 1 to 10 wherein the PBD comprises a PBD selected from the group consisting of PBD 1, PBD 2, PBD 3, PBD 4 and PBD 5.

12. The antibody drug conjugate of any of claims 1 to 11 wherein the antibody drug conjugate comprises a cleavable linker.

13. The antibody drug conjugate of claim 12 wherein the cleavable linker comprises a dipeptide.

14. A pharmaceutical composition comprising the antibody drug conjugate of any of claims 1 to 13 and a pharmaceutically acceptable carrier.

15. A method of treating cancer in a subject comprising administering to said subject a pharmaceutical composition of claim 14.

16. The method of claim 15 wherein the cancer comprises small cell lung cancer.

17. A method of preparing an antibody drug conjugate of any of claims 1-13 comprising the steps of:
a) providing an anti-DLL3 antibody comprising an unpaired cysteine;
b) selectively reducing the anti-DLL3 antibody; and
c) conjugating the selectively reduced anti-DLL3 antibody to a PBD.

18. The method of claim 17 wherein the step of selectively reducing the anti-DLL3 antibody comprises the step of contacting the antibody with a stabilizing agent.

19. The method of any of claim claims 17 to 19 further comprising the step of purifying the antibody drug conjugate using preparative chromatography.

20. An antibody drug conjugate comprising an ADC selected from the group consisting of ADC 1, ADC 2, ADC 3, ADC 4 and ADC 5 wherein Ab comprises an engineered anti-DLL3 antibody.
